(19) United States
(12) Patent Application Publication
(10) Pub. No.: US 2023/0099421 A1
(43) Pub. Date: Mar. 30, 2023

(54) A NEW METHOD OF 18F LABELLING AND INTERMEDIATE SALTS

(71) Applicant: National University of Singapore, Singapore (SG)

(72) Inventors: Rowan Drury Young, Singapore (SG); Dipendu Mandal, Singapore (SG); Richa Gupta, Singapore (SG); Amit Kumar Jaiswal, Singapore (SG)

(73) Assignee: National University of Singapore, Singapore (SG)

(21) Appl. No.: 17/757,653

(22) PCT Filed: Dec. 15, 2020
(86) PCT No.: PCT/SG2020/050749

§ 371 Date: Jun. 17, 2022

(30) Foreign Application Priority Data
Dec. 17, 2019 (SG) 10201912437Q

(51) Int. Cl. C07D 213/04; C07D 405/10; C07F 7/08; C07F 7/10; C07F 9/54; C07F 9/655; C07F 9/58; C07C 22/08; C07C 247/10; C07C 331/24; C07C 203/04; C07C 43/225; C07C 69/635; C07C 323/09; C07C 17/093; C07C 201/02; C07C 41/16; C07C 67/11; C07C 319/14; C07B 59/00; B01J 31/02

(52) U.S. Cl. CPC C07D 213/04; C07D 405/10; C07F 7/0812; C07F 7/10; C07F 9/5442; C07F 9/65515; C07F 9/58; C07C 22/08; C07C 247/10; C07C 331/24; C07C 203/04; C07C 43/225; C07C 69/635; C07C 323/09; C07C 17/093; C07C 201/02; C07C 41/16; C07C 67/11; C07C 319/14; C07B 59/001; C07B 59/004; C07B 59/002; B01J 31/0275; C07B 2200/05

(57) ABSTRACT

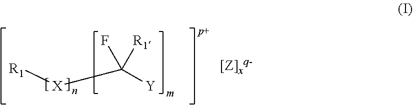

Disclosed herein is a salt of formula I: where R¹, X, n, R, R¹, Y, m, p, q, Z and o are as defined herein. Also disclosed herein are methods of using said salts in chemical synthesis, such as to prepare compounds isotopically enriched in 18F for use in PET & imaging, as well as methods to make the compounds of formula I.

### FIELD OF INVENTION

The current invention relates to specific salts and their uses in various reactions, particularly the formation of final products that are enriched with ¹⁸F.

### BACKGROUND

The listing or discussion of a prior-published document in this specification should not necessarily be taken as an acknowledgement that the document is part of the state of the art or is common general knowledge.

Fluorinated compounds have a wide range of applications in materials, agrochemistry and, most importantly, medicinal chemistry. The introduction of a single fluorine atom or a fluorine-containing motif into organic compounds significantly modify their physicochemical characteristics. Thus, incorporating fluorine atoms into drug candidates to improve pharmacological properties has become a common strategy in drug design and is fuelled by the number and efficacy of fluorine-containing drugs in the pharmaceutical market. This has also driven researchers to develop new synthetic methods to access a wider variety of organofluorine compounds.

However, the substitution of a single fluorine atom in trifluoromethylarenes remains an enduring challenge, as a generic method for this process has not yet been reported. C—F activation is difficult due to the high bond strength of C—F bond, yet it is this quality that makes fluorine substitution attractive for developing pharmaceuticals and agrochemicals. In addition, the ease of fluorine substitution in a CF₃ group increases as geminal fluorine atoms are substituted, resulting in poor control of the substitution reaction. Examples of monoselective C—F functionalization via single electron transfer and transition metal catalysis exist for benzotrifluorides but they are limited by their scope and/or coupling partners. As a result of limited synthetic methods, the potential of fluorine-containing motifs with high chemical diversity in the biologically relevant 3D chemical space cannot be fully exploited.

Besides drug development, the inclusion of radioactive ¹⁸F isotopes into organic drugs are valuable for positron emission topography (PET) imaging. PET is a useful diagnostic and pharmacological imaging tool that provides information on drug deposition and occupancy.

Although fluorine has several isotopes, the favourable half-life (109.8 min) and positron emission property of ¹⁸F make ¹⁸F attractive for PET imaging and thus ¹⁸F labelled radiotracers are routinely employed in PET for molecular imaging for early detection of diseases and treatment response. The two general methods to incorporate a ¹⁸F atom into organic compounds are: direct substitution of ¹⁸F atom; and indirect substitution via a ¹⁸F labelled prosthetic group. Unfortunately, only a small number of ¹⁸F labelled radiotracers have entered clinical trials due to various limitations, especially of current methods. For example, given the short half-life of ¹⁸F, the synthesis and purification needs to be conducted rapidly just before use. Unfortunately, current methods do not allow for the general, rapid synthesis of materials containing ¹⁸F isotopes.

CF₃ is a popular fluorine-containing motif for PET because it can significantly influence the physicochemical characteristics of a drug. In addition, it has the potential to enable ¹⁸F labelling of many drugs that contain a CF₃ group already. Given that these drugs already contain a CF₃ group, the introduction of a ¹⁸F atom will not affect the drugs' structural integrity or activity. Thus, fluoride substitution in CF₃ groups could give rise to a new generation of radiotracers. However, the few existing methods to access ¹⁸F labelled CF₃ groups are often non-selective and result in the substitution of multiple fluorides (i.e. the substitution of multiple ¹⁹F atoms with ¹⁸F). Furthermore, the inclusion of ¹⁸F in current methods is not performed as the final synthetic step, which results in a lower specific activity of ¹⁸F, due to the short half-life of this isotope.

Therefore, there exists a need to seek new methodologies for selective and facile accessibility of fluorine-containing and ¹⁸F labelled drugs for PET scan purposes to bring further advancement in drug discovery and diagnostics.

### SUMMARY OF INVENTION

It has been surprisingly found that the salts described herein enable the facile insertion of fluorine atoms into a wide variety of substrates. The process enables high product yields with fast reaction times, without the need to use expensive metals, such as stoichiometric amounts of gold. This process may be used to manufacture ¹⁸F-enriched materials that may be used for therapeutic and/or diagnostic purposes, where the synthesis and use of the desired material needs to be conducted over a short time scale is essential.

Aspects and embodiments of the invention are summarised in the following numbered clauses.
1. A salt of formula I:

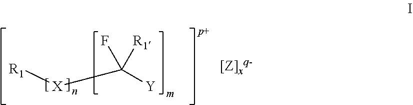

wherein:

m and p are 1 to 6;

n is 0 or 1;

q is 1 or 2 and o is 1 to 6, where Z is one or more counterions that balance the charge p+;

X, when present, is O, S or NR^{2a}R^{2b};

Y is —NR^{3a}R^{3b}R^{3c} or —PR^{4a}R^{4b}R^{4c};

R¹ is selected from H, alkyl, alkenyl, alkynyl, heterocyclic, aryl, or heteroaryl, which groups are unsubstituted or substituted by one or more groups selected from:
(a) halo;
(b) CN;
(c) C₁₋₆ alkyl, C₂₋₆ alkenyl, C₂₋₆ alkynyl (which latter three groups are unsubstituted or substituted by one or more substituents selected from halo, nitro, CN, C₁₋₆ alkyl, C₂₋₆ alkenyl, C₂₋₆ alkynyl (which latter three groups are unsubstituted or substituted by one or more substituents selected from OH, ═O, halo, C₁₋₄ alkyl and C₁₋₄ alkoxy), Cy¹ (which Cy¹ group is unsubstituted or is substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{5a}, S(O)_{q}R^{5b}, S(O)₂NR^{5c}R^{5d}, NR^{5e}S(O)₂R^{5f}, NR^{5g}R^{5b}, aryl and Het¹);
(d) Cy² (which Cy² group is unsubstituted or is substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{6a}, S(O)_{q}R^{6b}, S(O)₂NR^{6c}R^{6d}, NR^{6e}S(O)₂R^{6f}, NR^{6g}R^{6h}, aryl and Het²),
(e) Het^{a} (which Het^{a} group is unsubstituted or substituted by one or more substituents selected from halo, nitro, CN, C₁₋₆alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{7a}, S(O)_{q}R^{7b}, S(O)₂NR^{7c}R^{7d}, NR^{7e}S(O)₂R^{7f}, NR^{7g}R^{7h}, aryl and Het³);
(f) OR^{8a};
(g) S(O)_{q}R^{8b};
(h) S(O)₂NR^{8c}R^{8d};
(i) NR^{8e}S(O)₂R^{8f};
(j) NR^{8g}R^{8h},

R^{3a} to R^{3b} and R^{4a} to R^{4c} are each independently selected from aryl or heteroaryl, or R^{3a} to R^{3c} together form a pyridinium ring, which groups are unsubstituted or substituted by one or more groups selected from:
(a) halo;
(b) CN;
(c) C₁₋₆ alkyl, C₂₋₆ alkenyl, C₂₋₆ alkynyl (which latter three groups are unsubstituted or substituted by one or more substituents selected from halo, nitro, CN, C₁₋₆ alkyl, C₂₋₆ alkenyl, C₂₋₆ alkynyl (which latter three groups are unsubstituted or substituted by one or more substituents selected from OH, ═O, halo, C₁₋₄ alkyl and C₁₋₄ alkoxy), Cy³ (which Cy³ group is unsubstituted or is substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{9a}, S(O)_{q}R^{9b}, S(O)₂NR^{9c}R^{9d}, NR^{9e}S(O)₂R^{9f}, NR^{9g}R^{9h}, aryl and Het⁴);
(d) Cy⁴ (which Cy⁴ group is unsubstituted or is substituted by one or more substituents selected from halo, nitro, CN, C₁₋₆alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{10a}, S(O)_{q}R^{10b}, S(O)₂NR^{10c}R^{10d}, NR^{10e}S(O)₂R^{10f}, NR^{10g}R^{10h}, aryl and Het⁵),
(e) Het^{b} (which Het^{b} group is unsubstituted or substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{12a}, S(O)_{q}R^{12b}, S(O)₂NR^{12c}R^{12d}, NR^{12e}S(O)₂R^{12f}NR^{12g}R^{12h}, aryl and Het⁶);
(f) OR^{13a};
(g) S(O)_{q}R^{13b};
(h) S(O)₂NR^{13c}R^{13d};
(i) NR^{8e}S(O)₂R^{13f};
(j) NR^{13g}R^{13f},

R^{2a}, R^{2b}, R^{5a} to R^{5h}, R^{6a} to R^{6h}, R^{7a} to R^{7h}, R^{6a} to R^{8h}, R^{9a} to R^{9h}, R^{10a} to R^{10h}, R^{11a} to R^{11h}, R^{12a} to R^{12h}, and R^{13a} to R^{13h} independently represent, at each occurrence, H, C₁₋₆alkyl, C₂₋₆alkenyl, C₂₋₆ alkynyl (which latter three groups are unsubstituted or are substituted by one or more substituents selected from halo, nitro, ═O, C(O)OC₁₋₄ alkyl, CN, C₁₋₆ alkyl, C₂₋₆ alkenyl, C₂₋₆ alkynyl, C₃₋₆ cycloalkyl (which latter four groups are unsubstituted or are substituted by one or more substituents selected from OH, ═O, halo, C₁₋₄ alkyl and C₁₋₄ alkoxy), OR^{14a}, S(O)_{q}R^{14b}, S(O)₂NR^{14c}R^{14d}, NR^{14e}S(O)₂R^{14f}, NR^{14g}R^{14h}, aryl and Het⁷), C₄₋₁₀ cycloalkyl, or 0C₄₋₁₀ cycloalkenyl (which latter two groups are unsubstituted or are substituted by one or more substituents selected from halo, OH, ═O, C₁₋₆ alkyl and C₁₋₆ alkoxy) or Het^{c}, or
R^{2a} and R^{2b}R^{5-14c} and R^{5-14d}, and R^{5-14g} and R^{5-14h} represent, together with the nitrogen atom to which they are attached, a 3- to 10-membered heterocyclic ring that may be aromatic, fully saturated or partially unsaturated and which may additionally contain one or more heteroatoms selected from O, S and N, which heterocyclic ring is unsubstituted or are substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy);
Het¹ to Het⁶, Het^{a} to Het^{c} independently represent a 4- to 14-membered heterocyclic groups containing one or more heteroatoms selected from O, S and N, which heterocyclic groups may comprise one, two or three rings and may be substituted by one or more substituents selected from ═O, or more particularly, halo, C₁₋₆ alkyl, which latter group is unsubstituted or is substituted by one or more substituents selected from halo, —OR^{15a}, —NR^{15b}R^{15c}, —C(O)OR^{15d} and —C(O)NR^{15e}R^{15f};
Cy¹ to Cy⁴, at each occurrence, independently represents a 3- to 10-membered aromatic, fully saturated or partially unsaturated carbocyclic ring;
R^{15a} to R^{15h} independently represent at each occurrence, H, C₁₋₄ alkyl, C₂₋₄ alkenyl, C₂₋₄ alkynyl which latter three groups are unsubstituted or are substituted by one or more substituents selected from halo, nitro, CN, C₁₋₄alkyl, C₂₋₄ alkenyl, C₂₋₄ alkynyl (which latter three groups are unsubstituted or are substituted by one or more substituents selected from OH, ═O, halo, C₁₋₄alkyl and C₁₋₄alkoxy), C₃₋₆cycloalkyl, or C₄₋₆ cycloalkenyl (which latter two groups are unsubstituted or are substituted by one or more substituents selected from halo, OH, ═O, C₁₋₄ alkyl and C₁₋₄ alkoxy);

R¹′ is F, H aryl or alkyl, provided that when R¹′ is H, aryl or alkyl then Y is —NR^{3a}R^{3b}R^{3c}
2. The salt of formula I according to Clause 1, wherein:

m and p are 1 to 3;

n is 0 or 1;

q is 1 and o is 1 to 3; and

X, when present, is O or S.
3. The salt of formula I according to Clause 1 or Clause 2, wherein:

R¹ is selected from H, C₁₋₆ alkyl, C₂₋₆ alkenyl, C₂₋₆ alkynyl, heterocyclic, aryl, or heteroaryl, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, C₁₋₄ alkyl, C₂₋₄ alkenyl, C₂₋₄ alkynyl (which latter three groups are unsubstituted or substituted by one or more substituents selected from OH, ═O, halo, C₁0.3 alkyl and C₁₋₃ alkoxy), Cy¹ (which Cy¹ group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{5a}, S(O)_{q}R^{5b}, S(O)₂NR^{5c}R^{5d}, NR^{5e}S(O)₂R^{5f}, NR^{5g}R^{5f}, aryl and Het¹);
(d) Cy² (which Cy² group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{6a}, S(O)_{q}R^{6b}, S(O)₂NR^{6c}R^{6d}, NR^{6e}S(O)₂R^{6f}, NR^{6g}R^{6f}, aryl and Het²),
(e) Het^{a} (which Het^{a} group is unsubstituted or substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{7a}, S(O)_{q}R^{7b}, S(O)₂NR^{7c}R^{7d}, NR^{7e}S(O)₂R^{7f}, NR^{7g}R^{7h}, aryl and Het³);
(f) OR^{8a};
(g) S(O)_{q}R^{8b};
(h) S(O)₂NR^{8c}R^{8d};
(i) NR^{8e}S(O)₂R^{8f};
(j) NR^{8g}R^{8h}.
4. The salt of formula I according to Clause 3, wherein:

R¹ is selected from C₁₋₆ alkyl, aryl, or heteroaryl, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, unsubstituted C₁₋₄ alkyl, Cy¹ (which Cy¹ group is unsubstituted or is substituted by one or more substituents selected from halo, nitro, CN, unsubstituted C₁₋₄alkyl, OR^{5a}, and NR^{5g}R^{5h});
(d) Cy² (which Cy² group is unsubstituted or is substituted by one or more substituents selected from halo, nitro, CN, unsubstituted C₁₋₄alkyl, OR^{6a}, and NR^{6g}R^{6h})
(e) Het^{a} (which Het^{a} group is unsubstituted or substituted by one or more substituents selected from halo, nitro, CN, unsubstituted C₁₋₄alkyl, OR^{7a}, and NR^{7g}R^{7h});
(f) OR^{8a};
(g) NR^{8g}R^{8h}, optionally, wherein

R¹ is selected from C₁₋₆ alkyl, phenyl, or pyridyl, which groups are unsubstituted or substituted by one or more groups as described in any one of Clauses 1, 3 and 4.
5. The salt of formula I according to any one of the preceding clauses, wherein: R^{3a} to R^{3b} and R^{4a} to R^{4c} are each independently selected from aryl or heteroaryl, or R^{3a} to R^{3c} together form a pyridinium ring, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, C₁₋₄ alkyl, C₂₋₄ alkenyl, C₂₋₄ alkynyl (which latter three groups are unsubstituted or substituted by one or more substituents selected from OH, ═O, halo, C₁₋₃ alkyl and C₁₋₃ alkoxy), Cy³ (which Cy³ group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{9a}, S(O)_{q}R^{9b}, S(O)₂NR^{9c}R^{9d}, NR^{9e}S(O)₂R^{9f}, NR^{9g}R^{9h}, aryl and Het⁴);
(d) Cy⁴ (which Cy⁴ group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{10a}, S(O)_{q}R^{10b}, S(O)₂NR^{10c}R^{10d}, NR^{10e}S(O)₂R^{10f}, NR^{10g}R^{10h}, aryl and Het⁵),
(e) Het^{b} (which Het^{b} group is unsubstituted or substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁0.3 alkyl and C₁0.3 alkoxy), OR^{12a}, S(O)_{q}R^{12b}, S(O)₂NR^{12c}R^{12d}, NR^{12e}S(O)₂R^{12f}, NR^{12g}R^{12f}, aryl and Het⁶);
(f) OR^{13a};
(g) S(O)_{q}R^{13b};
(h) S(O)₂NR^{13c}R^{13d};
(i) NR^{8e}S(O)₂R^{13f};
(j) NR^{13g}R^{13h}.
6. The salt of formula I according to Clause 5, wherein:

R^{3a} to R^{3c} and R^{4a} to R^{4c} are each independently selected from aryl or heteroaryl, or R^{3a} to R^{3c} together form a pyridinium ring, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, unsubstituted C₁₋₄ alkyl, Cy³ (which Cy³ group is unsubstituted or is substituted by one or more substituents selected from halo, nitro, CN, unsubstituted C₁₋₄alkyl, OR^{9a}, and NR^{9g}R^{9h});
(d) Cy⁴ (which Cy⁴ group is unsubstituted or is substituted by one or more substituents selected from halo, nitro, CN, unsubstituted C₁₋₄ alkyl, OR^{10a}, S(O)_{q}R^{10b}, S(O)₂NR^{10c}R^{10d}, NR^{10e}S(O)₂R^{10f}, NR^{10g}R^{10h}, aryl and Het⁵),
(e) Het^{b} (which Het^{b} group is unsubstituted or substituted by one or more substituents selected from halo, nitro, CN, unsubstituted C₁₋₄ alkyl, OR^{12a}, and NR^{12g}R^{12h}); (f) OR^{13a};
(g) NR^{13g}R^{13h}.
7. The salt of formula I according to any one of the preceding clauses, wherein, when present:

R^{2a} and R^{2b}, R^{5a} to R^{5h}, R^{6a} to R^{6h}, R^{7a} to R^{7h}, R^{8a} to R^{8h}, R^{9a} to R^{9h}, R^{10a} to R^{10h}R^{11a} to R^{11h}, R^{12a} to R^{12h}, and R^{13a} to R^{13h} independently represent, at each occurrence, H or C₁₋₄ alkyl (which is unsubstituted or is substituted by one or more substituents selected from halo, nitro, ═O, CN, unsubstituted C₁₋₄alkyl, OR^{14a}, and NR^{14g}R^{14h}), or
R^{2a} and R^{2b}R^{5-14c} and R^{5-14d}, and R^{5-14g} and R^{5-14h} represent, together with the nitrogen atom to which they are attached, a 3- to 10-membered heterocyclic ring that may be aromatic, fully saturated or partially unsaturated and which may additionally contain one or more heteroatoms selected from O, S and N, which heterocyclic ring is unsubstituted or are substituted by one or more substituents selected from halo, nitro, CN, or C₁₋₆ alkyl.
8. The salt of formula I according to any one of the preceding clauses, wherein, when present:
Het¹ to Het⁶, Het^{a} to Het^{c} independently represent a 4— to 10-membered heterocyclic groups containing one or more heteroatoms selected from O, S and N, which heterocyclic groups may comprise one, two or three rings and may be substituted by one or more substituents selected from ═O, or more particularly, halo, C₁₋₄ alkyl, which latter group is unsubstituted or is substituted by one or more substituents selected from halo, —OR^{15a}, —NR^{15b}R^{15c}, —C(O)OR^{15d} and —C(O)NR^{15e}R^{15f};
Cy¹ to Cy⁴, at each occurrence, independently represents a 3— to 8-membered aromatic, fully saturated or partially unsaturated carbocyclic ring;
R^{15a} to R^{15h} independently represent at each occurrence, H, C₁₋₄ alkyl, which group is unsubstituted or is substituted by one or more substituents selected from halo, nitro, CN, or unsubstituted C₁₋₄ alkyl.
9. The salt of formula I according to any one of the preceding clauses, wherein Y is —NR^{3a}R^{3b}R^{3c}.
10. The salt of formula I according to any one of Clauses 1 to 8, wherein Y is selected from:

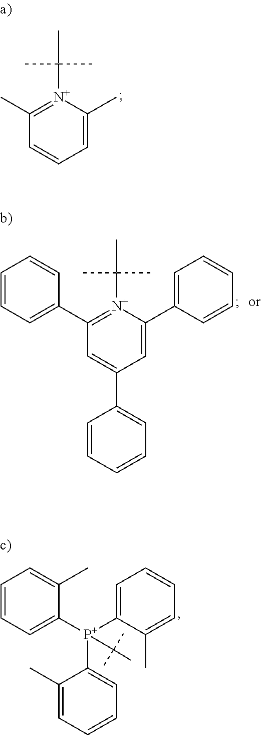

where the dotted line represents the point of attachment to the rest of the molecule.
11. The salt of formula I according to any one of the preceding clauses, wherein Y is:

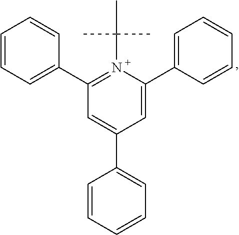

where the dotted line represents the point of attachment to the rest of the molecule.
12. The salt of formula I according to any one of the preceding clauses, wherein:
(a) Z is selected from one or more of B—(C₆F₅)₄, FB—(C₆F₅)₃ or, more particularly, N—(SO₂CF₃)₂; and/or
(b) R¹ is F.
13. The salt of formula I according to any one of the preceding clauses, selected from the list of:

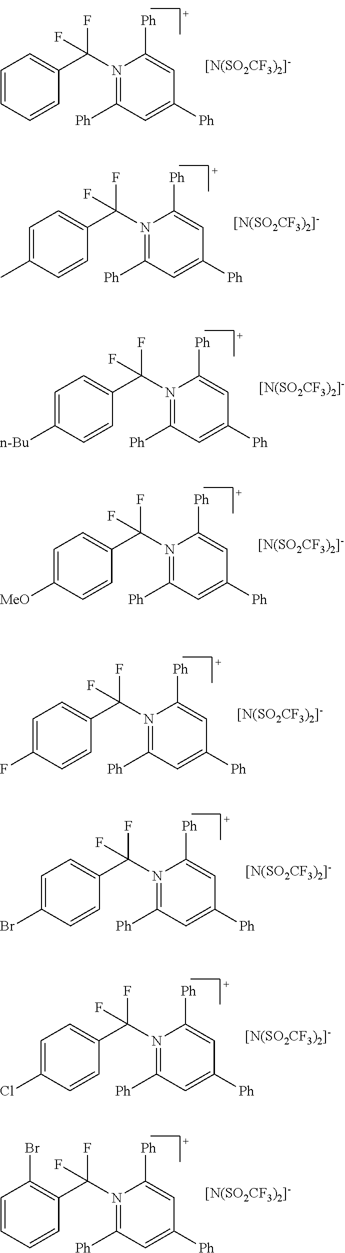

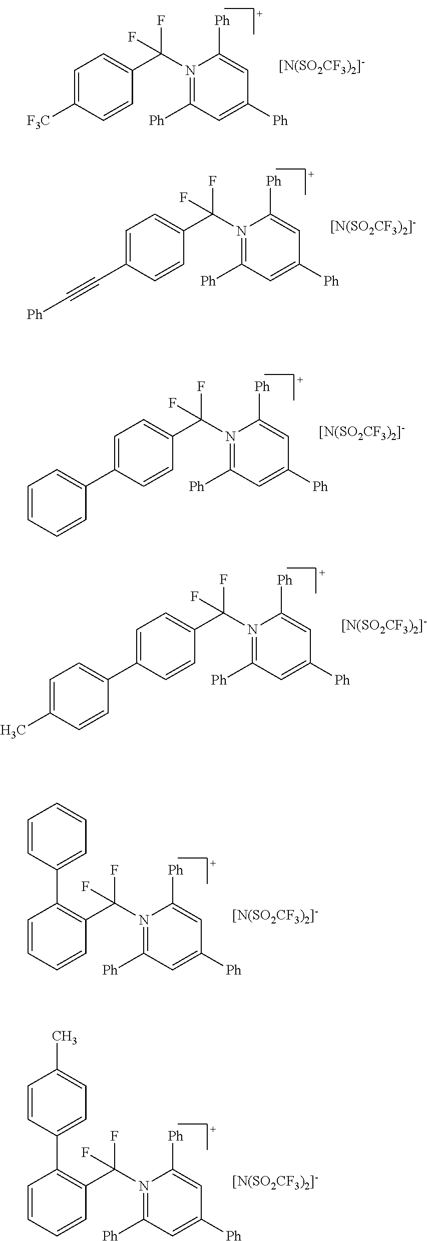

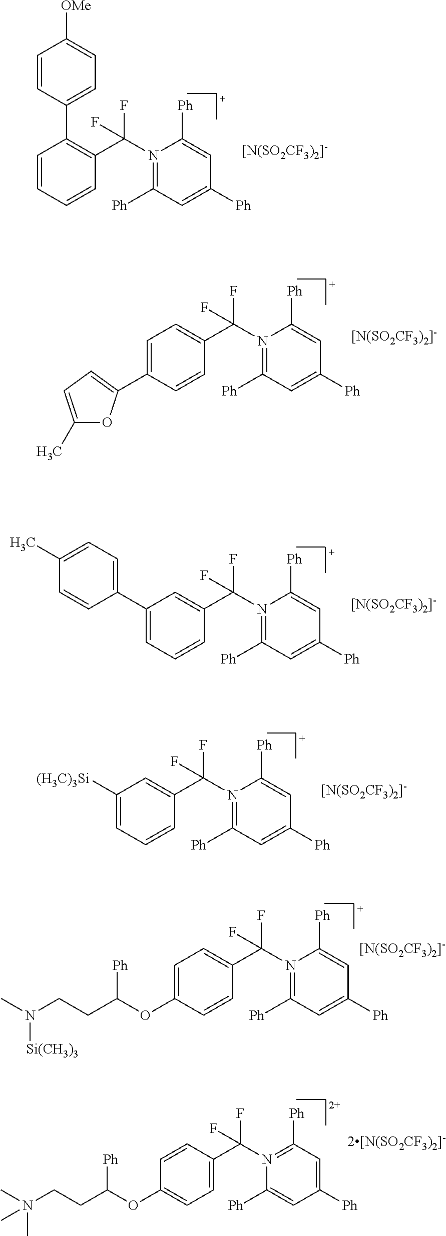

optionally wherein salt of formula I according to any one of the preceding clauses, selected from the list of:

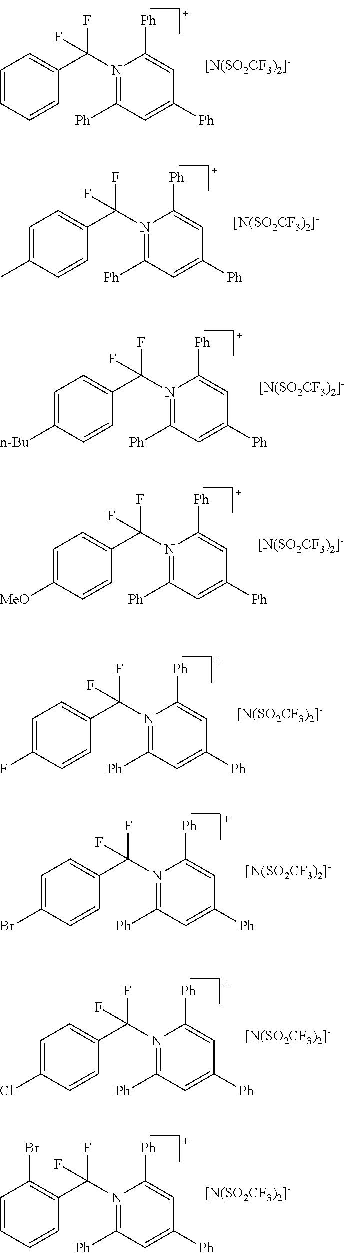

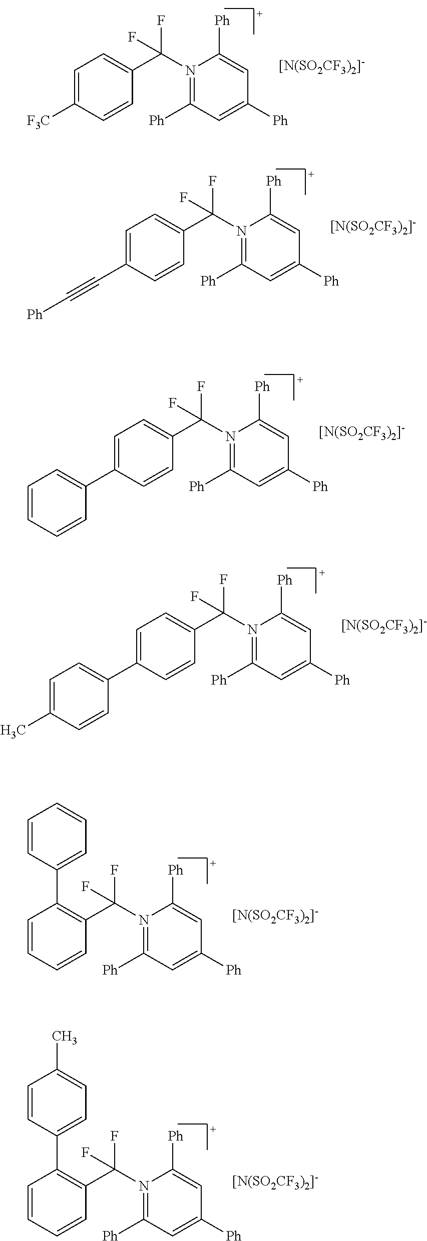

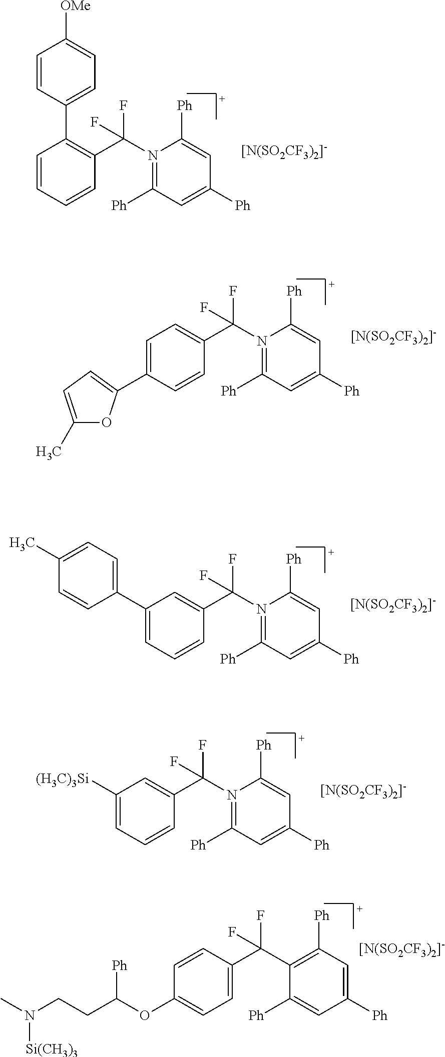

such as from the list:

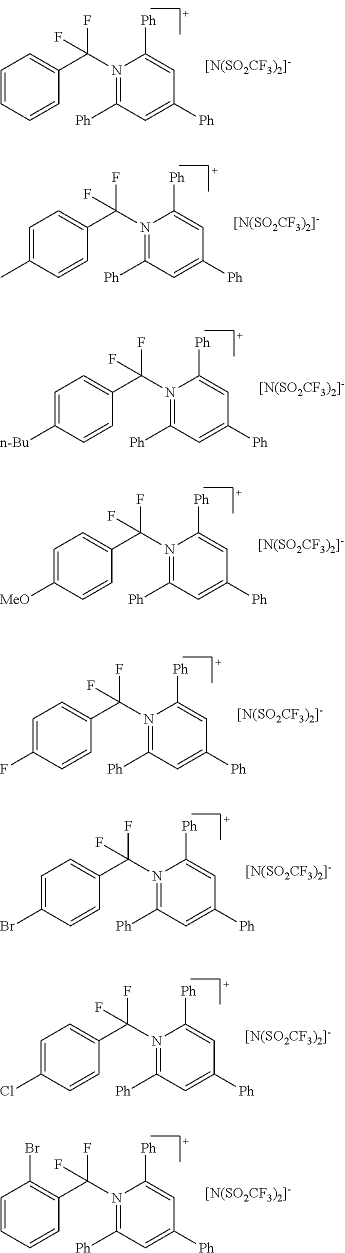

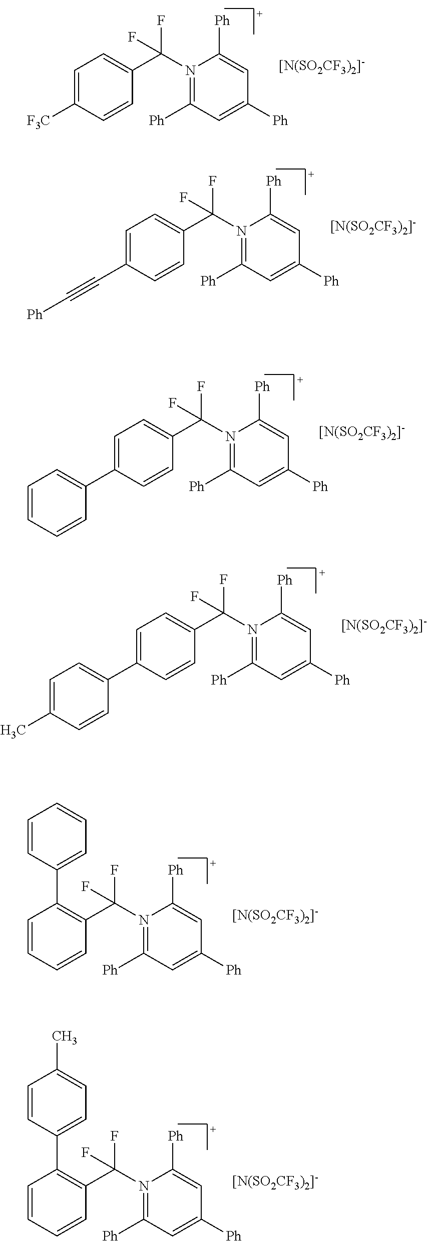

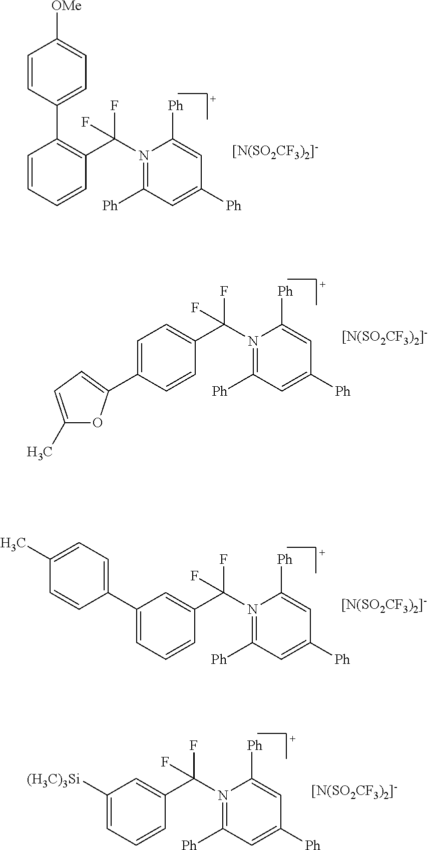

14. A method of forming a compound of formula I as described in any one of Clauses 1 to 13, the method comprising the step of reacting a compound of formula II,

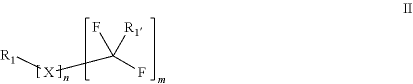

with a compound of formula IIIa or IIIb:

NR^{3a}R^{3b}R^{3c} IIIa; or

PR^{4a}R^{4b}R^{4c} IIIb,

in the presence of a catalyst and a counterion source, where n, m, R¹, R^{3a} to R^{3b} and R^{4a} to R^{4b} are as described in any one of Clauses 1 to 13, provided that when R¹′ is H, aryl or alkyl, then the reaction is with a compound of formula IIIa.
15. The method according to Clause 14, wherein:
(a) the counterion source is selected from Li[B(C₆F₅)₄] or, more particularly, N-(trimethylsilyl)bis(trifluoromethanesulfonyl)imide; and/or
(b) the catalyst is selected from B(C₆F₅)₃.
16. A method of providing a difluorinated compound with or without an isotopic label, comprising the step of reacting a compound of formula I as described in any one of Clauses 1 to 13, with a nucleophilic source compound with or without an isotopic label to form the difluorinated compound.
17. A one-pot method of providing a difluorinated compound with or without an isotopic label from a compound of formula as described in Clause 14, the method comprising the steps of:
(a) reacting a compound of formula II with a compound of formula IIIa or IIIb in the presence of a catalyst and a counterion source to provide a compound of formula I, provided that when R¹′ is H, aryl or alkyl, then the reaction is with a compound of formula IIIa, where the compounds of formula I, IIIa and IIIb are as described in Clause 14 and the compound of formula I is as described in any one of Clauses 1 to 13; and
(b) reacting a compound of formula I as described in any one of Clauses 1 to 13, with a nucleophilic source compound with or without an isotopic label to form the difluorinated compound.
19. The method of Clause 16 or the method of Clause 17, wherein the nucleophilic source compound is selected from one or more of the group consisting of Bn(Et₃)NCl, (nBu)₄NBr, (nBu)₄NI, (nBu)₄N¹⁸F, NaN₃, (nBu)₄NSCN, NaNO₃, sodium phenoxides (e.g. sodium 2-bromophenolate, sodium 4-methoxyphenolate), sodium thiophenols (e.g. sodium thiphenol, sodium 4-methylthiuophenol), pyridines (e.g. pyridine or 2,6-lutidine), triphenyl phosphines (e.g. triphenyl phosphine, P(oTol)₃), and sodium esters (e.g. NaOAc).
20. A method of forming a difluorinated compound through nucleophilic difluorination, the method comprising the step of reacting a compound of formula I as described in any one of Clauses 1 to 13 with a compound having an thioaldehyde group, a thioketone group or, more particularly, aldehyde group, a ketone group or an imine group in the presence of an initiator compound to form a difluorinated compound, optionally wherein the initiator compound is selected from an inorganic base, such as, but not limited to CsCO₃, KOH, NaOH and the like.
21. A method of forming either a difluorinated compound through a radical coupling reaction to an alkene, alkyne or hydrogen, the method comprising reacting a compound of formula I as described in Clause 1 with an alkene or alkyne or hydrogen source in the presence of a radical initiator to generate the difluorinated compound.

### DRAWINGS

FIG. 1A-E illustrates the molecular structures of 2a, 2g, 3b, 4k and 3m obtained by X-Ray Crystallography. Hydrogen atoms and anions are omitted and thermal ellipsoids shown at 50%. A: Compound 2a; B: Compound 2g; C: Compound 3b; D: Compound 4k; and E: Compound 3m, (phenyl rings on TPPy substituent is shown in wire frame. Short distance [C8-N1:3.066(4) Å] shows π-π stacking between the eclipsed ortho aryl group and the pyridinium moiety).

FIG. 2A-B represents the radiochemical purity and radiochemical yield of isolated [¹⁸F]-trifluorotoluene. A: HPLC analysis of isolated [¹⁸F]-trifluorotoluene (top: UV profile; and bottom: gamma profile); and B: Relevant data for HPLC analysis.

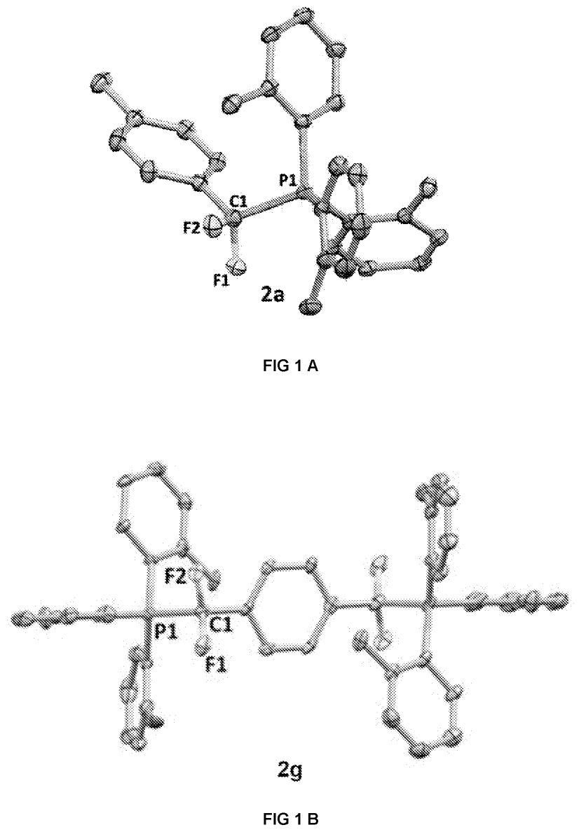

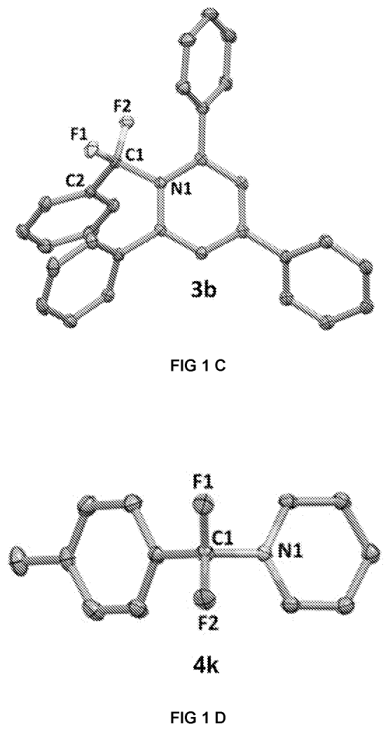

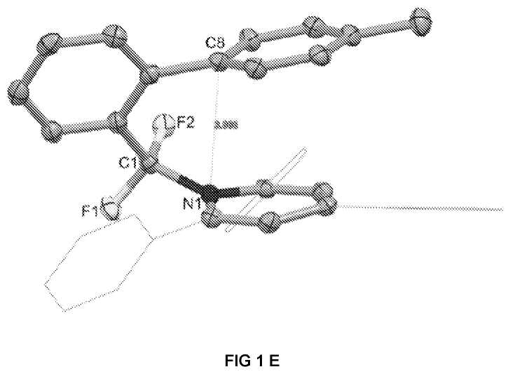

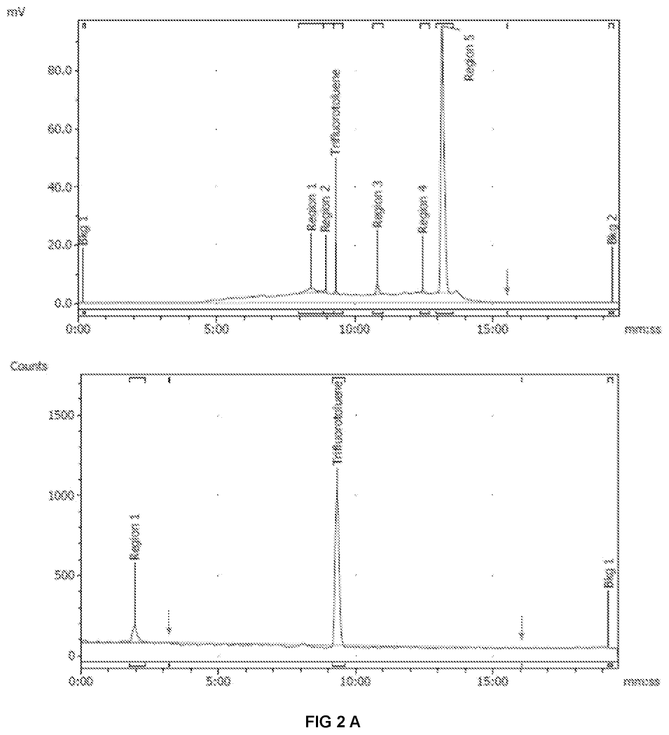

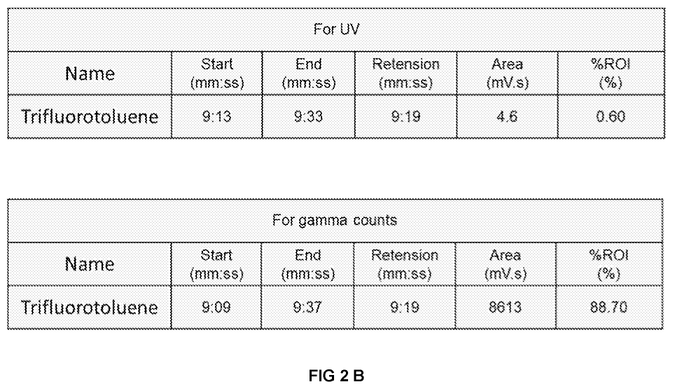

### DESCRIPTION

As noted hereinbefore, it has been surprisingly found that certain salts can be used as a substrate for a rapid, high-yielding synthesis of tri-fluorinated final compounds that may be enriched with the ¹⁸F isotope. This process has also been unexpectedly found to work for the production of di-fluorinated species too.

Thus, in a first aspect of the invention, there is provided a salt of formula I:

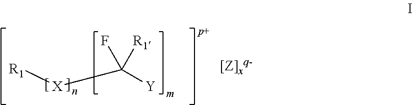

wherein:

m and p are 1 to 6;

n is 0 or 1;

q is 1 or 2 and o is 1 to 6, where Z is one or more counterions that balance the charge p+;

X, when present, is O, S or NR^{2a}R^{2b};

Y is —NR^{3a}R^{3b}R^{3c} or —PR^{4a}R⁴OR^{4c};

R¹ is selected from H, alkyl, alkenyl, alkynyl, heterocyclic, aryl, or heteroaryl, which groups are unsubstituted or substituted by one or more groups selected from:
(a) halo;
(b) CN;
(c) C₁₋₆alkyl, C₂₋₆ alkenyl, C₂₋₆ alkynyl (which latter three groups are unsubstituted or substituted by one or more substituents selected from halo, nitro, CN, C₁₋₆alkyl, C₂₋₆ alkenyl, C₂₋₆ alkynyl (which latter three groups are unsubstituted or substituted by one or more substituents selected from OH, ═O, halo, C₁₋₄ alkyl and C₁₋₄ alkoxy), Cy¹ (which Cy¹ group is unsubstituted or is substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{5a}, S(O)_{q}R^{5b}, S(O)₂NR^{5c}R^{5d}, NR^{5e}S(O)₂R^{5f}, NR^{5g}R^{5f}, aryl and Het¹);
(d) Cy² (which Cy² group is unsubstituted or is substituted by one or more substituents selected from halo, nitro, CN, C₁₋₆alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{6a}, S(O)_{q}R^{6b}, S(O)₂NR^{6c}R^{6d}, NR^{6e}S(O)₂R^{6f}, NR^{6g}R^{6f}, aryl and Het²),
(e) Het^{a} (which Het^{a} group is unsubstituted or substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{7a}, S(O)_{q}R^{7b}, S(O)₂NR^{7c}R^{7d}, NR^{7e}S(O)₂R^{7f}, NR^{7g}R^{7h}, aryl and Het³);
(f) OR^{8a};
(g) S(O)_{q}R^{8b};
(h) S(O)₂NR^{8c}R^{8d};
(i) NR^{8e}S(O)₂R^{8f};
(j) NR^{8g}R^{8h}

R^{3a} to R^{3c} and R^{4a} to R^{4c} are each independently selected from aryl or heteroaryl, or R^{3a} to R^{3c}together form a pyridinium ring, which groups are unsubstituted or substituted by one or more groups selected from:
(a) halo;
(b) CN;
(c) C₁₋₆ alkyl, C₂₋₆ alkenyl, C₂₋₆ alkynyl (which latter three groups are unsubstituted or substituted by one or more substituents selected from halo, nitro, CN, C₁₋₆ alkyl, C₂₋₆ alkenyl, C₂₋₆ alkynyl (which latter three groups are unsubstituted or substituted by one or more substituents selected from OH, ═O, halo, C₁₋₄ alkyl and C₁₋₄ alkoxy), Cy³ (which Cy³ group is unsubstituted or is substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{9a}, S(O)_{q}R^{9b}, S(O)₂NR^{9c}R^{9d}, NR^{9e}S(O)₂R^{9f}, NR^{9g}R^{9h}, aryl and Het⁴);
(d) Cy⁴ (which Cy⁴ group is unsubstituted or is substituted by one or more substituents selected from halo, nitro, CN, C₁₋₆alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{10a}, S(O)_{q}R^{10b}, S(O)₂NR^{10c}R^{10d}, NR^{10e}S(O)₂R^{10f}, NR^{10g}R^{10h}, aryl and Het⁵),
(e) Het^{b} (which Het^{b} group is unsubstituted or substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{12a}, S(O)_{q}R^{12b}, S(O)₂NR^{12c}R^{12d}, NR^{12e}S(O)₂R^{12f}, NR^{12g}R^{12h}, aryl and Het⁶);
(f) OR^{13a};
(g) S(O)_{q}R^{13b};
(h) S(O)₂NR^{13c}R^{13d};
(i) NR^{8e}S(O)₂R^{13f};
(j) NR^{13g}R^{13f},

R^{2a}, R^{2b}, R^{5a} to R^{5h}, R^{6a} to R^{6h}, R^{7a} to R^{7h}, R^{8a} to R^{8h}, R^{9a} to R^{9h}, R^{10a} to R^{10h}R^{11a} to R^{11h}, R^{12a} to R^{12h}, and R^{13a} to R^{13h} independently represent, at each occurrence, H, C₁₋₆alkyl, C₂₋₆alkenyl, C₂₋₆ alkynyl (which latter three groups are unsubstituted or are substituted by one or more substituents selected from halo, nitro, ═O, C(O)OC₁₋₄ alkyl, CN, C₁₋₆ alkyl, C₂₋₆ alkenyl, C₂₋₆ alkynyl, C₃₋₆ cycloalkyl (which latter four groups are unsubstituted or are substituted by one or more substituents selected from OH, ═O, halo, C₁₋₄ alkyl and C₁₋₄ alkoxy), OR^{14a}, S(O)_{q}R^{14b}, S(O)₂NR^{14c}R^{14d}, NR^{14e}S(O)₂R^{14f}, NR^{14g}R^{14h}, aryl and Het⁷), C₃₋₁₀ cycloalkyl, or C₄₋₁₀ cycloalkenyl (which latter two groups are unsubstituted or are substituted by one or more substituents selected from halo, OH, ═O, C₁₋₆ alkyl and C₁₋₆ alkoxy) or Het^{c}, or
R^{2a} and R^{2b}R^{5-14c} and R^{5-14d}, and R^{5-14g} and R^{5-14h} represent, together with the nitrogen atom to which they are attached, a 3— to 10-membered heterocyclic ring that may be aromatic, fully saturated or partially unsaturated and which may additionally contain one or more heteroatoms selected from O, S and N, which heterocyclic ring is unsubstituted or are substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy);
Het¹ to Het⁶, Het^{a} to Het^{c} independently represent a 4— to 14-membered heterocyclic groups containing one or more heteroatoms selected from O, S and N, which heterocyclic groups may comprise one, two or three rings and may be substituted by one or more substituents selected from ═O, or more particularly, halo, C₁₋₆ alkyl, which latter group is unsubstituted or is substituted by one or more substituents selected from halo, —OR^{15a}, —NR^{15b}R^{15c}, —C(O)OR^{15d} and —C(O)NR^{15e}R^{15f};
Cy¹ to Cy⁴, at each occurrence, independently represents a 3— to 10-membered aromatic, fully saturated or partially unsaturated carbocyclic ring;
R^{15a} to R^{15h} independently represent at each occurrence, H, C₁₋₄ alkyl, C₂₋₄ alkenyl, C₂₋₄ alkynyl which latter three groups are unsubstituted or are substituted by one or more substituents selected from halo, nitro, CN, C₁₋₄alkyl, C₂₋₄ alkenyl, C₂₋₄ alkynyl (which latter three groups are unsubstituted or are substituted by one or more substituents selected from OH, ═O, halo, C₁₋₄alkyl and C₁₋₄alkoxy), C₃₋₆cycloalkyl, or C₄0.6 cycloalkenyl (which latter two groups are unsubstituted or are substituted by one or more substituents selected from halo, OH, ═O, C₁₋₄ alkyl and C₁₋₄ alkoxy);
R¹′ is F, H aryl or alkyl, provided that when R¹′ is H, aryl or alkyl then Y is —NR^{3a}R^{3b}R^{3c}.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The term “halo”, when used herein, includes references to fluoro, chloro, bromo and iodo.

Unless otherwise stated, the term “aryl” when used herein includes C₆₋₁₄ (such as C₆₋₁₀) aryl groups. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Embodiments of the invention that may be mentioned include those in which aryl is phenyl.

Unless otherwise stated, the term “alkyl” refers to an unbranched or branched, cyclic, saturated or unsaturated (so forming, for example, an alkenyl or alkynyl) hydrocarbyl radical, which may be substituted or unsubstituted (with, for example, one or more halo atoms). Where the term “alkyl” refers to an acyclic group, it is preferably C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₁₂ cycloalkyl and, more preferably, C₅0.10 (e.g. C₅₇) cycloalkyl.

The term “heteroaryl” when used herein refers to an aromatic group containing one or more heteroatom(s) (e.g. one to four heteroatoms) preferably selected from N, O and S (so forming, for example, a mono-, bi-, or tricyclic heteroaromatic group). Heteroaryl groups include those which have between 5 and 14 (e.g. 10) members and may be monocyclic, bicyclic or tricyclic, provided that at least one of the rings is aromatic. However, when heteroaryl groups are bicyclic or tricyclic, they are linked to the rest of the molecule via an aromatic ring. Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Particularly preferred heteroaryl groups include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, thiophenyl, pyranyl, carbazolyl, acridinyl, quinolinyl, benzoimidazolyl, benzthiazolyl, purinyl, cinnolinyl and pterdinyl. Particularly preferred heteroaryl groups include monocylic heteroaryl groups.

Further embodiments of the invention that may be mentioned include those in which the compound of formula I is isotopically labelled. However, other, particular embodiments of the invention that may be mentioned include those in which the compound of formula I is not isotopically labelled.

The term “isotopically labelled”, when used herein includes references to compounds of formula I and, particularly, compounds of formula II (as described below), in which there is a non-natural isotope (or a non-natural distribution of isotopes) at one or more positions in the compound. References herein to “one or more positions in the compound” will be understood by those skilled in the art to refer to one or more of the atoms of the compound of formula I and II. Thus, the term “isotopically labelled” includes references to compounds of formula I and II that are isotopically enriched at one or more positions in the compound.

The isotopic labelling or enrichment of the compound of formula I and II may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁷O, ¹⁸O, ³⁵S, ¹⁸F, ³⁷Cl, ⁷⁷Br, ⁸²Br and ¹²⁵I) When the compound of formula I and formula II is labelled or enriched with a radioactive or nonradioactive isotope, compounds of formula I and II that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

Examples of isotopic labelling that may be mentioned herein include the use of ¹⁸F to generate a C¹⁹F₂¹⁸F group (e.g. in compounds of formula II, as described below). Further examples of isotopic labelling that may be mentioned herein include the use of ¹⁸F to generate a C¹⁹F¹⁸FH group (e.g. in compounds of formula II, as described below).

As noted above, the salt of formula I may contain one or more cationic sections (m), each section defined by a cationic N⁺ or P⁺ ion. Thus, the values for m and p in the salt of formula I are tied together and will have the same value. The number of these cationic groups depends on what R¹ is. For example, if R¹ is H then m will be 1. However, if R¹ is a single carbon atom, then m may be from 1 to 4. When R¹ is a larger group with more possible substituents, then m may be from 1 to 6. Thus, for the avoidance of doubt, R¹ may be substituted from 1 to 6 substituents [X]ₙ-[CFR¹Y] (e.g. [X]ₙ-[CF₂Y]). It will be appreciated that [X]ₙ is either no present (when n=0) or represents a covalent linking group between R¹ and the [CFR¹Y] group when n is 1.

As will be appreciated, the value of p will be balanced by one or more counterions Z. For example, when p is 4, and Z is a monovalent anion (where q is 1), then o will be 4. However, if Z is a dianion (where q is 2) then o will be 2.

In embodiments of the invention that may be mentioned herein, the salt of formula I may be one in which:

m and p are 1 to 3;

n is 0 or 1;

q is 1 and o is 1 to 3; and

X, when present, is O or S. For example, in certain embodiments that may be mentioned

herein, the salt of formula I may be one in which:

m and p are 1;

n is 0; and

q is 1 and o is 1.

In embodiments of the invention that may be mentioned herein, the salt of formula I may be one in which:

R¹ is selected from H, C₁₋₆ alkyl, C₂₋₆ alkenyl, C₂₋₆ alkynyl, heterocyclic, aryl, or heteroaryl, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, C₁₋₄ alkyl, C₂₋₄ alkenyl, C₂₋₄ alkynyl (which latter three groups are unsubstituted or substituted by one or more substituents selected from OH, ═O, halo, C₁0.3 alkyl and C₁₋₃ alkoxy), Cy¹ (which Cy¹ group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{5a}, S(O)_{q}R^{5b}, S(O)₂NR^{5c}R^{5d}, NR^{5e}S(O)₂R^{5f}, NR^{5g}R^{5f}, aryl and Het¹);
(d) Cy² (which Cy² group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{6a}, S(O)_{q}R^{6b}, S(O)₂NR^{6c}R^{6d}, NR^{6e}S(O)₂R^{6f}, NR^{6g}R^{6f}, aryl and Het²),
(e) Het^{a} (which Het^{a} group is unsubstituted or substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{7a}, S(O)_{q}R^{7b}, S(O)₂NR^{7c}R^{7d}, NR^{7e}S(O)₂R^{7f}, NR^{7g}R^{7h}, aryl and Het³);
(f) OR^{8a};
(g) S(O)_{q}R^{8b};
(h) S(O)₂NR^{8c}R^{8d};
(i) NR^{8e}S(O)₂R^{8f};
(j) NR^{8g}R^{8h} As will be appreciated, the total number of possible substituents in R¹ will depend on the valency of the R₁ group. For example, if R¹ is a n-propyl group, then the total number of potential substituents is eight. Thus, for a propyl group, the total possible number of substituents that may be selected from (a) to (j) hereinbefore may be a maximum of 8-m. As will be appreciated, not all (or any) of the potential positions that are available for substitution may be substituted (in which case, the position will be occupied by H).

For example, R¹ may be selected from C₁₋₆ alkyl, aryl, or heteroaryl, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, unsubstituted C₁₋₄ alkyl, Cy¹ (which Cy¹ group is unsubstituted or is substituted by one or more substituents selected from halo, nitro, CN, unsubstituted C₁₋₄ alkyl, OR^{5a}, and NR^{5g}R^{5h});
(d) Cy² (which Cy² group is unsubstituted or is substituted by one or more substituents selected from halo, nitro, CN, unsubstituted C₁₋₄alkyl, OR^{6a}, and NR^{6g}R^{6h})
(e) Het^{a} (which Het^{a} group is unsubstituted or substituted by one or more substituents selected from halo, nitro, CN, unsubstituted C₁₋₄alkyl, OR^{7a}, and NR^{7g}R^{7h});
(f) OR^{8a};
(g) NR^{8g}R^{8h}.

In particular embodiments that may be mentioned herein, R¹ may be selected from C₁₋₆ alkyl, phenyl, or pyridyl, which groups are unsubstituted or substituted by one or more groups as described hereinbefore.

In embodiments of the invention that may be mentioned herein, R^{3a} to R^{3c} and R^{4a} to R^{4c} are each independently selected from aryl or heteroaryl, or R^{3a} to R^{3b} together form a pyridinium ring. For the avoidance of doubt, when each of R^{3a} to R^{3b} and R^{4a} to R^{4c} are aryl or heteroaryl, then they may be unsubstituted or substituted by substituents mentioned herein. In addition, when R^{3a} to R^{3b} together form a pyridinium ring, said pyridinium ring may be unsubstituted or substituted by the substituents mentioned herein. Thus, in embodiments of the invention, R^{3a} to R^{3b} and R^{4a} to R^{4c} may each be each independently selected from aryl or heteroaryl, or R^{3a} to R^{3b} together form a pyridinium ring, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, C₁₋₄ alkyl, C₂₋₄ alkenyl, C₂₋₄ alkynyl (which latter three groups are unsubstituted or substituted by one or more substituents selected from OH, ═O, halo, C₁0.3 alkyl and C₁₋₃ alkoxy), Cy³ (which Cy³ group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{9a}, S(O)_{q}R^{9b}, S(O)₂NR^{9c}R^{9d}, NR^{9e}S(O)₂R^{9f}, NR^{9g}R^{9h}, aryl and Het⁴);
(d) Cy⁴ (which Cy⁴ group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{10a}, S(O)_{q}R^{10b}, S(O)₂NR^{10c}R^{10d}, NR^{10e}S(O)₂R^{10f}, NR^{10g}R^{10h}, aryl and Het⁵),
(e) Het^{b} (which Het^{b} group is unsubstituted or substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁0.3 alkyl and C₁0.3 alkoxy), OR^{12a}, S(O)_{q}R^{12b}, S(O)₂NR^{12c}R^{12d}, NR^{12e}S(O)₂R^{12f}, NR^{12g}R^{12h}, aryl and Het⁶);
(f) OR^{13a};
(g) S(O)_{q}R^{13b};
(h) S(O)₂NR^{13c}R^{13d};
(i) NR^{8e}S(O)₂R^{13f};
(j) NR^{13g}R^{13h}.

In more particular examples disclosed herein, R^{3a} to R^{3b} and R^{4a} to R^{4c} may each independently be selected from aryl or heteroaryl, or R^{3a} to R^{3b} together form a pyridinium ring, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, unsubstituted C₁₋₄ alkyl, Cy³ (which Cy³ group is unsubstituted or is substituted by one or more substituents selected from halo, nitro, CN, unsubstituted C₁₋₄alkyl, OR^{9a}, and NR^{9g}R^{9h});
(d) Cy⁴ (which Cy⁴ group is unsubstituted or is substituted by one or more substituents selected from halo, nitro, CN, unsubstituted C₁₋₄ alkyl, OR^{10a}, S(O)_{q}R^{10b}, S(O)₂NR^{10c}R^{10d}, NR^{10e}S(O)₂R^{10f}, NR^{10g}R^{10h}, aryl and Het⁵),
(e) Het^{b} (which Het^{b} group is unsubstituted or substituted by one or more substituents selected from halo, nitro, CN, unsubstituted C₁₋₄ alkyl, OR^{12a}, and NR^{12g}R^{12h});
(f) OR^{13a};
(g) NR^{13g}R^{13h}.

In embodiments of the invention that may be mentioned herein, when present: R^{2a} and R^{2b}, R^{5a} to R^{5h}, R^{6a} to R^{6h}, R^{7a} to R^{7h}, R^{8a} to R^{8h}, R^{9a} to R^{9h}, R^{10a} to R^{10h}R^{11a} to R^{11h}, R^{12a} to R^{12h}, and R^{13a} to R^{13h} independently represent, at each occurrence, H or C₁₋₄ alkyl (which is unsubstituted or is substituted by one or more substituents selected from halo, nitro, ═O, CN, unsubstituted C₁₋₄alkyl, OR^{14a}, and NR^{14g}R^{14h}), or

R^{2a} and R^{2b}R^{5-14c} and R^{5-14d}, and R^{5-14g} and R^{5-14h} represent, together with the nitrogen atom to which they are attached, a 3— to 10-membered heterocyclic ring that may be aromatic, fully saturated or partially unsaturated and which may additionally contain one or more heteroatoms selected from O, S and N, which heterocyclic ring is unsubstituted or are substituted by one or more substituents selected from halo, nitro, CN, or C₁₋₆ alkyl.

In embodiments of the invention that may be mentioned herein, when present:
Het¹ to Het⁶, Het^{a} to Het^{c} independently represent a 4— to 10-membered heterocyclic groups containing one or more heteroatoms selected from O, S and N, which heterocyclic groups may comprise one, two or three rings and may be substituted by one or more substituents selected from ═O, or more particularly, halo, C₁₋₄ alkyl, which latter group is unsubstituted or is substituted by one or more substituents selected from halo, —OR^{15a}, —NR^{15b}R^{15c}, —C(O)OR^{15d} and —C(O)NR^{15e}R^{15f};
Cy¹ to Cy⁴, at each occurrence, independently represents a 3— to 8-membered aromatic, fully saturated or partially unsaturated carbocyclic ring;
R^{15a} to R^{15h} independently represent at each occurrence, H, C₁₋₄ alkyl, which group is unsubstituted or is substituted by one or more substituents selected from halo, nitro, CN, or unsubstituted C₁₋₄ alkyl.

In particular embodiments of the invention that may be mentioned herein, Y in the compound of formula I may be —NR^{3a}R^{3b}R^{3c}. In additional or alternative embodiments of the invention that may be mentioned herein, Y may be selected from:

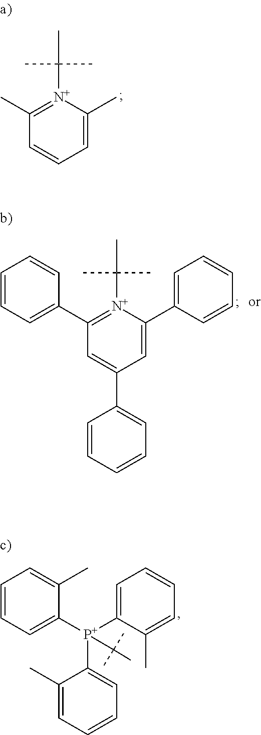

where the dotted line represents the point of attachment to the rest of the molecule.

In more particular embodiments of the invention that may be mentioned herein, the salt of formula I may be one in which Y is:

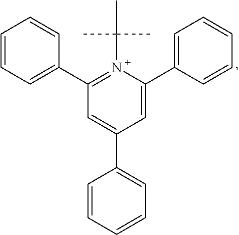

where the dotted line represents the point of attachment to the rest of the molecule.

In embodiments of the invention, the counterion of the salt of formula I may be one where Z is selected from one or more of B—(C₆F₅)₄, FB—(C₆F₅)₃ or, more particularly, N—(SO₂CF₃)₂.

In particular embodiments of the invention that may be mentioned herein, the compound of formula I may be one in which R¹ ′ is F.

Particular salts of formula I that may be mentioned herein include:

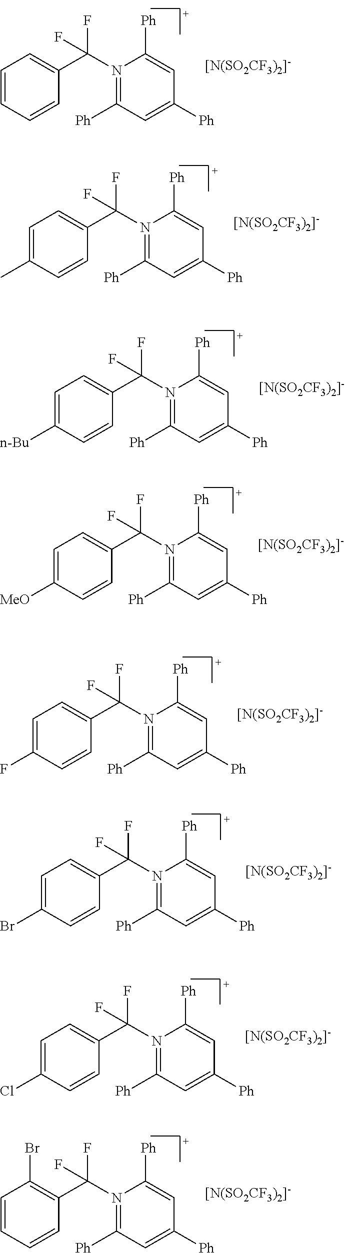

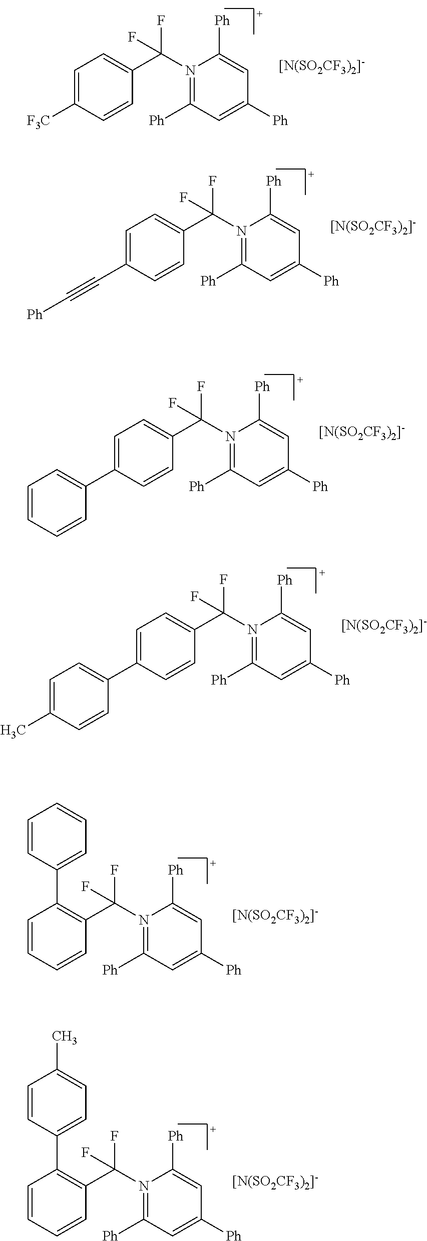

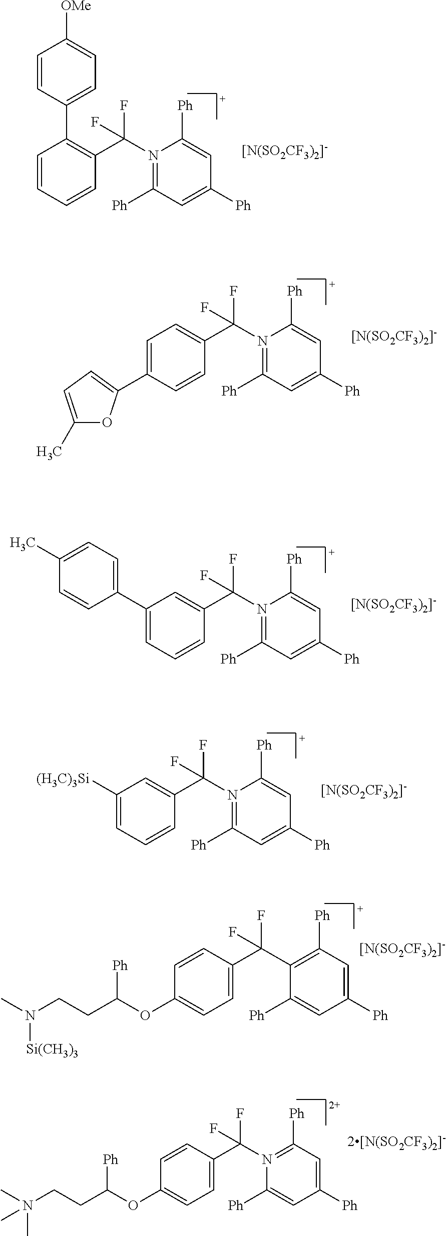

It will be appreciated that selections from this list may be made. For example, as depicted in Clause 13 of the Summary of invention.

Thus, in a further aspect of the invention, there is disclosed a method of forming a compound of formula I as described hereinbefore, the method comprising the step of reacting a compound of formula II,

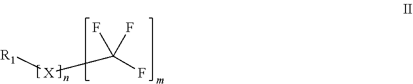

with a compound of formula IIIa or IIIb:

NR^{3a}R^{3b}R^{3c} IIIa; or

PR^{4a}R^{4b}R^{4c} IIIb,

in the presence of a catalyst and a counterion source, where n, m, R¹, R^{3a} to R^{3b} and R^{4a} to R^{4b} are as described hereinbefore, provided that when R¹ is H, aryl or alkyl, then the reaction is with a compound of formula IIIa.

Any suitable counterion source may be used in the reaction described above. Suitable counterion sources include, but are not limited to Li[B(C₆F₅)₄] and N-(trimethylsilyl)bis(trifluoromethanesulfonyl)imide. In particular embodiments of the invention that may be mentioned herein, the counterion source may be N-(trimethylsilyl)bis(trifluoromethanesulfonyl)imide.

Any suitable catalyst may be used in the reaction described above. For example, the catalyst may be B(C₆F₅)₃.

As will be appreciated, the salt of formula I may be used to form a difluorinated compound with, or without, an isotopic label. Thus, in a further aspect of the invention, there is provided a method of providing a difluorinated compound with or without an isotopic label, comprising the step of reacting a compound of formula I as described hereinbefore, with a nucleophilic source compound with or without an isotopic label to form the difluorinated compound.

The formation of the difluorinated compound discussed above may also be conducted in two steps, which may (or may not) be combined into one pot. In this embodiment, the method may comprise the steps of:
(a) reacting a compound of formula II with a compound of formula IIIa or IIIb in the presence of a catalyst and a counterion source to provide a compound of formula I, provided that when R¹′ is H, aryl or alkyl, then the reaction is with a compound of formula IIIa, where the compounds of formula II, IIa and IIIb are as described hereinbefore and the compound of formula I is as described hereinbefore; and
(b) reacting a compound of formula I as described hereinbefore, with a nucleophilic source compound with or without an isotopic label to form the difluorinated compound.

When the term, “provided that when R¹′ is H, aryl or alkyl” is used, it will be understood that the proviso only applies to compounds of formula I etc, where R¹′ is selected from these substituents. It is not intended to affect the scope of claims described herein where R¹′ is F. Thus, in particular embodiments of the invention that may be mentioned herein, R¹′ may be F.

As will be appreciated, the nature of the final compound will depend on the nature of the nucleophile. For example, if the nucleophile provides a F atom, then the resulting compound will be a trifluorinated compound. In such examples, it may be preferred that the nucleophile supplies a ¹⁹F atom. That is, the nucleophile used may be one where the nucleophile is enriched by ¹⁹F atoms. As will be appreciated, other nucleophiles used in the reaction mentioned herein may also be isotopically enriched.

It is noted that the formation of isotopically-enriched materials in a facile manner and in high yields remains a desirable goal. This is because many of the isotopes used may have a limited half-life, meaning that the isotopically labelled materials must be formed and used rapidly. Thus, it is believed that the salts of formula I disclosed herein enable a significantly improved reaction to access isotopically labelled compounds quickly and in high yield.

Any suitable nucleophile may be used in the reactions described herein. For example, the nucleophilic source compound may be selected from the group including, but not limited to, Bn(Et₃)NCl, (nBu)₄NBr, (nBu)₄NI, (nBu)₄N¹⁸F, NaN₃, (nBu)₄NSCN, NaNO₃, sodium phenoxides (e.g. sodium 2-bromophenolate, sodium 4-methoxyphenolate), sodium thiophenols (e.g. sodium thiphenol, sodium 4-methylthiuophenol), pyridines (e.g. pyridine or 2,6-lutidine), triphenyl phosphines (e.g. triphenyl phosphine, P(oTol)₃), sodium esters (e.g. NaOAc), and combinations thereof.

In addition, the salts of formula I may also allow easy access to other structural motifs that remain difficult to access using conventional synthetic strategies. Thus, the salts of formula I may also enable access to these structural motifs, which will now be discussed below and expanded upon in the examples.

The salts of formula I may also be reacted with aldehydes, ketone and their equivalents to by a nucleophilic transfer reaction to provide difluorinated final compounds. For example, when the compound of formula I is reacted with an aldehyde, the resulting product is an alcohol (see examples section for more detail). Thus, in a further aspect of the invention, there is provided a method of forming a difluorinated compound through nucleophilic difluorination, the method comprising the step of reacting a compound of formula I as described hereinbefore with a compound having a thioaldehyde group, a thioketone group or, more particularly, aldehyde group, a ketone group or an imine group in the presence of an initiator compound to form a difluorinated compound. Any suitable initiator compound may be used in this reaction. For example, the initiator compound may be an inorganic base. Examples of inorganic bases that may be used as the initiator compound include, but are to limited to CSCO₃, KOH, NaOH and the like. Any suitable a compound having a thioaldehyde group, a thioketone group or, more particularly, aldehyde group, a ketone group or an imine group may be used in this reaction and this is not particularly limited.

The salts of formula I may also be reacted may also undergo radical coupling reactions. Thus, in a further aspect of the invention, there is also provided a method of forming a difluorinated compound through a radical coupling reaction to an alkene, alkyne or hydrogen, the method comprising reacting a compound of formula I as described hereinbefore with an alkene or alkyne or hydrogen source in the presence of a radical initiator to generate the difluorinated compound. Any suitable alkene or alkyne or hydrogen source may be used in this reaction and this is not particularly limited.

Further aspects and embodiments of the invention will now be described by reference to the following non-limiting examples.

### EXAMPLES

Materials

Dichloromethane (DCM, CH₂Cl₂) and n-hexane were purified using an LC Technology Solution Inc. SP-1 Solvent Purification System, deoxygenated and stored over 4 Å molecular sieves prior to use. Chloroform-d (CDCl₃), dichloromethane-d₂ (CD₂Cl₂), 1,2-dichlorobenzene (1,2-DCB, 1,2-C₆H₄Cl₂), 1,2-dichloroethane (1,2-DCE, 1,2-C₂H4Cl₂), dibromomethane (DBM, CH₂Br₂), dimethylacetamide (DMA, C₄H9NO), dimethylformamide (DMF, C₃H₇NO) solvents were stirred over CaH₂ at RT under nitrogen atmosphere overnight prior to distillation under reduced pressure. Tetrahydrofuran (THF, C₄H₈O) was distilled under nitrogen from sodium and benzophenone and stored over 4 Å molecular sieves. Starting materials B(C₆F5)₃ (A. G. Massey, et aL., J. Organomet. Chem., 1964, 2, 245-250) and [AI(C₆F5)₃·0.5C₇H₈] (S. Feng, et al., Organometallics, 2002, 21, 832-839) (ACF) were prepared using reported methods. Trifluorides 1a-1w were prepared using reported methods or purchased from commercial sources. All other reagents were obtained commercially and used as received.

General

Experiments were performed under inert conditions using standard Schlenk techniques or a glove box (Vacuum Atmospheres Company) as appropriate. Subsequent manipulation of airstable products was carried out under ambient conditions. Column chromatography using silica (230-400 mesh) was carried out using analytical grade eluent mixtures of n-hexane and ethyl acetate. HRMS spectra were obtained using an Agilent Technologies 6230 TOF MS (ESI-TOF) and a Bruker micrOTOF-Q (APCI-TOF). X-Ray diffraction analysis was performed by Dr Hendrik Tinnermann at NUS Department of Chemistry. X-ray data were measured on a Bruker D8 Venture dual source diffractometer. The crystal structures were solved by direct methods using SHELXS-97 and refined with SHELXL-2014 using Olex3. ¹H, ¹³C, ¹⁹F and ³¹P NMR spectra were recorded at 298 K using Bruker AV-400 and AV-500 spectrometers. The chemical shifts (δ) for ¹H and ¹³C spectra are given in ppm relative to solvent signals, and ³¹P{¹H} and ¹⁹F{¹H} spectra were referenced to external 85% H₃PO₄, CFCl₃ standards, respectively. The [¹⁸F]-fluoride labelling experiment was carried out at Clinical Imaging Research Centre at National University of Singapore.

Preparation 1-Preparation of Cs[BF(C₆F₅)₃]

A DCM (1.0 mL) solution of B(C₆F₅)₃(0.113 g, 0.22 mmol, 1.1 equiv.) was added to C₆F (0.031 g, 0.2 mmol, 1.0 equiv.) in 1.0 mL DCM. After addition a turbidity appeared and for completion the reaction mixture left to stir at RT for 18 hours. Following filtration, hexane wash (3×2 mL) and drying afforded white powder of Cs[BF(C₆F₅)₃] (0.119 g, 90% yield).

¹⁹F NMR (377 MHz, DMSO-d₆): δF-134.6 (m, 6 F, o-C₆F₅), −160.6 (m, 3 F, p-C₆F₅), −165.5 (m, 6 F, m-C₆F₅), −190.0 (brs, 1 F, FB(C₆F₅)₃); ¹¹B NMR (128 MHz, DMSO-d₆): δ_{B}-0.86 (d, 1B, J=65.0 Hz, FB(C₆F₅)₃). HRMS (ESI-TOF) m/z: 529.9874 for [C₁₈BF₁₆]⁻ (calcd.: 529.9879).

Preparation 2-Formation of B(C₆F₅)₃ (BCF)

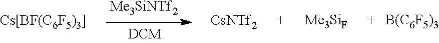

Cs[BF(C₆F₅)₃] (0.010 g, 0.015 mmol, 1.0 equiv.) was taken in dry DCM (0.5 mL). Me₃SiNTf₂ (0.005 g, 0.015 mmol, 1.0 equiv.) was transferred to the reaction mixture. After shaking for 5 minutes, ¹⁹F NMR was recorded. The ¹⁹F NMR chemical shifts of the formed BCF was confirmed by comparison to the literature and so as in authentic sample of BCF (see A. Massey, et aL., J. Organomet. Chem., 1964, 2, 245-250).

### Example 1: Optimisation Conditions for C—F Activation

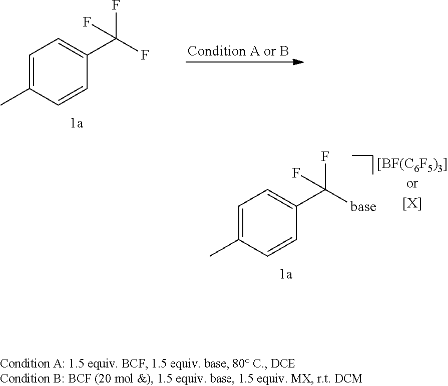

In a 4 mL open PTFE top screw cap vial catalyst (x mol %), trifluoride 1a (0.15 mmol, 1.0 equiv.) and MX (0.23 mmol, 1.5 equiv.) were added. Base (0.23 mmol, 1.5 equiv.) was dissolved in 300 μL dry solvent and transferred to the vessel. The reaction mixture was monitored and the reaction yield was assessed by ¹⁹F NMR analysis using an internal PhF or PhOCF₃ standard. This protocol was then used in multiple experiments seeking to obtain optimised conditions for the displacement of F from 1a. The varying conditions used and yields of the desired product are summarised in Table.

Results and Discussion

Treating 1a with BCF and P(o-tol)₃ at RT did not produce the desired phosphonium salt, while heating at 80° C. for 24 h only increased the yield to <1% (Table 1, entry 1). It was found that the removal of fluoride via precipitation of LiF or loss of Me₃SiF gas promotes the reaction and allows for a catalytic amount of BCF to be used. Therefore, Me₃SiNTf₂ was added to the reaction and was found to be effective even at RT, allowing high yields of the desired phosphonium salt 2a to be generated (Table 1, entries 5-7). Attempts to use tetrahydrothiophene, pyridine or lutidine as the base partner gave poor conversion or no reaction with 1a (Table 1, entries 8-10). On the other hand, the nitrogen donor base 2,4,6-triphenylpyridine (TPPy) generated the desired TPPy pyridinium salt, 3a, almost quantitatively at room temperature after 48 h (60% yield after 24 h) with catalytic loadings of BCF (Table 1, entry 12). Heating of the reaction mixtures containing TPPy led to a faster conversion of 1a to 3a (Table 1, entry 13), but resulted in the decomposition of 3a thus compromising the reaction yield. Finally, running the reaction without any BCF catalyst failed to generate any 3a (Table 1, entry 14) hence highlighting the importance of BCF.

TABLE 1

| | Catalyst | | | | | | | | |
|---|---|---|---|---|---|---|---|---|---|
| Entry | (mol %) | Base | Time | | MX | Solvent | Temperature | Yield (%) | Remarks |
| 1 | BCF (150%) | P(o-Tol)₃ | 24 | h | — | DCM | RT | 0 | No consumption of 1a |
| 2 | BCF (150%) | P(o-Tol)₃ | 24 | h | — | 1,2-DCE | 80° C. | <1 | Trace amount of phos salt formed |
| 3 | BCF (150%) | TPPy | 24 | h | — | 1,2-DCE | 80° C. | <1 | Trace amount TPPy-salt formed |
| 4 | BCF (20%) | P(o-Tol)₃ | 24 | h | Li[BF₄] | 1,2-DCE | 80° C. | 0 | No consumption of 1a |
| 5 | BCF (20%) | P(o-Tol)₃ | 24 | h | Li[B(C₆F₅)₄] | 1,2-DCE | 80° C. | 20 | 20% consumption of 1a. <5% [BCF—F]⁻ |
| | | | | | | | | | observed. |
| 6 | BCF (20%) | P(o-Tol)₃ | 24 | h | Me₃SiOTf | 1,2-DCE | 80° C. | 0 | No consumption of 1a |
| 7 | BCF (20%) | P(o-Tol)₃ | 24 | h | Me₃SiNTf₂ | DCM | RT | 70 | 75% consumption of 1a |
| 8 | BCF (5%) | P(o-Tol)₃ | 24 | h | Me₃SiNTf₂ | 1,2-DCE | 80° C. | 86 | 1.1 equiv. P(o-Tol)₃, 1.1 equiv. TMSNTf₂; |
| 9 | BCF (20%) | P(o-Tol)₃ | 4 | h | Me₃SiNTf₂ | 1,2-DCE | 80° C. | >95 | 100% consumption of 1a |
| 10 | BCF (20%) | TPPy | 48 | h | Me₃SiNTf₂ | DCM | RT | >95 | 100% consumption of 1a |
| 11 | BCF (20%) | TPPy | 24 | h | Me₃SiNTf₂ | DCM | 40° C. | 89 | 100% consumption of 1a |
| 12 | BCF (20%) | TPPy | 4 | h | Me₃SiNTf₂ | 1,2-DCE | 60° C. | 87 | 100% consumption of 1a; the TPPy-salt |
| | | | | | | | | | decomposes for overheating at 60° C. |
| 13 | — | TPPy | 48 | h | Me₃SiNTf₂ | 1,2-DCE | RT | 0 | No consumption of 1a |
| 14 | — | TPPy | 48 | h | Me₃SiNTf₂ | 1,2-DCE | 80° C. | <1 | Trace amount of TPPy-salt formed |
| 15 | BCF (20%) | Pyridine | 24 | h | Me₃SiNTf₂ | 1,2-DCE | 80° C. | 0 | No consumption of 1a |
| 16 | BCF (20%) | 2,6-Lutidine | 48 | h | Me₃SiNTf₂ | DCM | RT | <20 | 40% consumption of 1a. |
| 17 | BCF (20%) | Et₃N | 48 | h | Me₃SiNTf₂ | DCM | RT | 0 | No consumption of 1a |
| 18 | BCF (20%) | iPr₂NEt | 48 | h | Me₃SiNTf₂ | DCM | RT | 0 | No consumption of 1a |
| 19 | BCF (20%) | THT | 48 | h | Me₃SiNTf₂ | DCM | 40° C. | 0 | No consumption of 1a |
| 20 | BCF (20%) | Me₂S | 48 | h | Me₃SiNTf₂ | DCM | 40° C. | 0 | No consumption of 1a |
| 21 | BCF (20%) | NPh₃ | 48 | h | Me₃SiNTf₂ | DCM | 40° C. | 0 | No consumption of 1a |
| 22 | BF₃•OEt₂ | TPPy | 48 | h | Me₃SiNTf₂ | DCM | RT | 0 | No consumption of 1a |
| | (20%) | | | | | | | | |
| 20 | ACF (20%) | TPPy | 48 | h | Me₃SiNTf₂ | 1,2-DCB | RT | 63 | 63% consumption of 1a |
| 21 | [F₂P(C₆F₅)₃] | TPPy | 48 | h | Me₃SiNTf₂ | 1,2-DCE | 80° C. | <1 | The catalyst decomposes; observed trace |
| | (20%) | | | | | | | | amount of the TPPy-salt may be from |
| | | | | | | | | | TMSNTf₂ |
| 22 | BCF (20%) | TPPy | 48 | h | Me₃SiOTf | DCM | RT | 0 | No consumption of 1a |
| 23 | BCF (20%) | TPPy | 48 | h | Me₃SiOMs | DCM | RT | 0 | No consumption of 1a |
| 24 | BCF (20%) | TPPy | 48 | h | Me₃SiNTf₂ | DCM | RT | 50 | Solvent 600 μL |

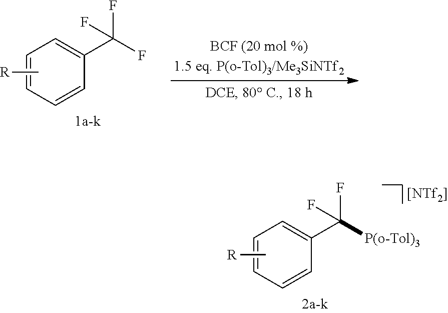

### Example 2: Method for NMR-Scale Synthesis of Phosphonium Salts, 2a-k

Into a 4 mL open PTFE top screw cap vial BCF (0.015 g, 0.03 mmol, 20 mol %), P(o-Tol)₃ (0.070 g, 0.23 mmol, 1.5 equiv.) and Me₃SiNTf₂ (0.081 g, 0.23 mmol, 1.5 equiv.) were added. After addition of a single compound selected from 1a-k (0.15 mmol. 1.0 equiv.) dissolved in 300 μL dry 1,2-DCE or 1,2-DCB as appropriate, the reaction mixture was allowed to heat and stirred. Reaction completion was monitored by ¹⁹F NMR analysis of the crude reaction mixture with an internal PhF or PhOCF₃ standard. The reaction yield was assessed by ¹⁹F NMR analysis and summarised in Table 2.

TABLE 2
NMR yields for benzotrifluoride scope using P(o-Tol)₃ base

| | | | | | | Tem- | | |
|---|---|---|---|---|---|---|---|---|
| En- | | | | | Solvent | pera- | Yield | |
| try | Substrate | Product | Step | Time | (amount) | ture | (%) | Remarks |
| 1 | 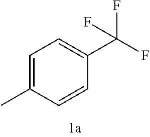 | 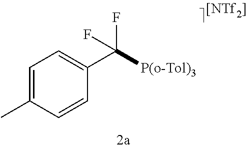 | step1 | 18 h | 1,2-DCE (300 μL) | 80° C. | >95 | Complete consumption of 1a |
| 2 | 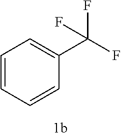 | 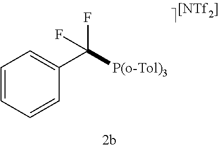 | step1 | 18 h | 1,2-DCE (300 μL) | 80° C. | >95 | Complete consumption of 1b |
| 3 | 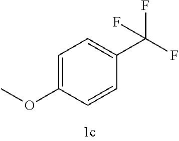 | 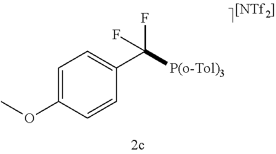 | step 1 | 4 h | 1,2-DCE (300 μL) | 80° C. | 79 | 100% consumption of 1c; yield was assessed in THF as chemical resonance for 2c overlaps NTf₂ ¹⁹F NMR signal in 1,2- |
| | | | | | | | | DCE. |
| 4 | 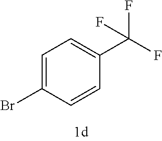 [SMILES: FC(F)(F)c1ccc(Br)cc1] | 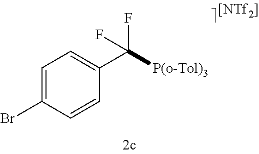 | step 1 | 24 h | 1.2-DCE (300 μL) | 80° C. | >95 | Complete consumption of 1d |
| 5 | 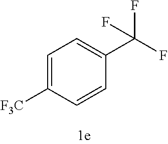 [SMILES: FC(F)(F)c1ccc(C(F)(F)F)cc1] | 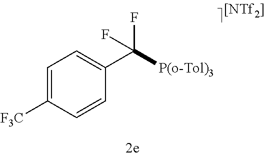 | step 1 | 48 h | 1,2-DCE (150 μL) | 80° C. | 35 | |
| 6 | 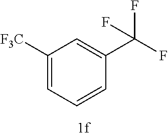 [SMILES: FC(F)(F)c1cccc(C(F)(F)F)c1] | 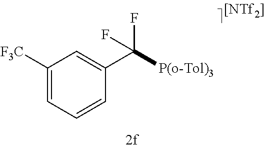 | step 1 | 48 h | 1,2-DCE (150 μL) | 80° C. | 40 | |
| 7 | 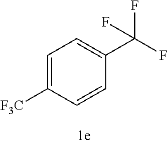 [SMILES: FC(F)(F)c1ccc(C(F)(F)F)cc1] | 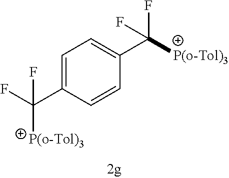 | step 1 | 16 h | 1,2-DCB (150 μL) | 150° C. | 71 | 100% consumption of 1e. |
| 8 | 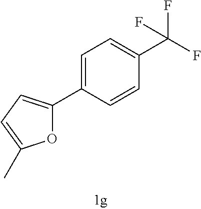 [SMILES: Cc1ccc(-c2ccc(C(F)(F)F)cc2)o1] | 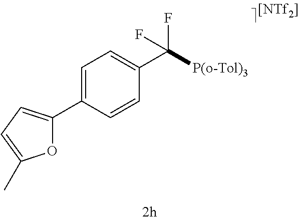 | step 1 | 4 h | 1,2-DCE (300 μL) | 80° C. | 61 | 99% consumption of 1g; yield was assessed in THF as chemical resonance for 2h overlaps NTF₂ ¹⁹F NMR signal in 1,2- DCE. |
| 9 | 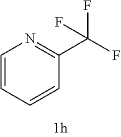 | 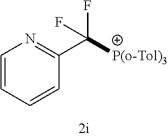 | step 1 | 48 h | 1,2-DCB (300 μL) | 150° C. | 48 | Complete consumption of 1h; yield was assessed using ³¹P NMR as chemical signal for 2i overlaps NTf₂ ¹⁹F NMR signal |
| | | | | | | | | in 1,2-DCE and |
| | | | | | | | | THF. |
| 10 | 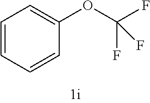 [SMILES: FC(F)(F)Oc1ccccc1] | 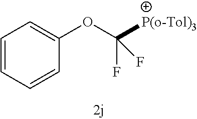 | step 1 | 60 h | 1,2-DCB (300 μl) | 100° C. | 50 | Complete consumption of 1i |
| 11 | 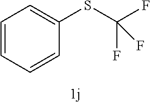 [SMILES: FC(F)(F)Sc1ccccc1] | 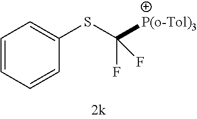 | step 1 | 18 h | 1,2-DCB (300 μL) | 150° C. | 71 | Complete consumption of 1j |

### Example 3: One-Pot Synthesis from Trifluoride to CF₂(Nucleophile) by S_{N}2 Functionalization

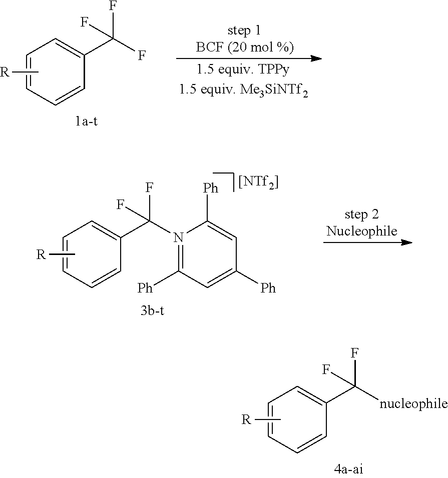

Procedure A

Into a 4 mL open PTFE top screw cap vial BCF (0.015 g, 0.03 mmol, 20 mol %), a trifluoride selected from compounds 1a-t (0.15 mmol. 1.0 equiv.), and Me₃SiNTf₂ (0.081 g, 0.23 mmol, 1.5 equiv.) were added. A solution of TPPy (0.070 g, 0.23 mmol, 1.5 equiv.) dissolved in 300 μL of dry DCM, DBM, 1,2-DCE or 1,2-DCB as appropriate was used for step 1 unless otherwise specified. After which, an appropriate source of nucleophile (0.38 mmol, 2.5 equiv.) taken in suitable 300 μL dry solvent was transferred to the reaction mixture. Reaction yields for step 1 and step 2 at different conditions were monitored by ¹⁹F NMR with an internal PhOCF₃ standard and these are summarised in Table 3, which lists results obtained using procedure A.

Procedure B

In subsequent studies, it was discovered that by changing the reagents' order of addition, it was possible to improve the yields of the desired products by up to 10-20%. Thus, the procedure below may be used in place of that used to generate the results in Table 3.

A trifluoride selected from compounds 1a-t (0.15 mmol. 1.0 equiv.) and Me₃SiNTf₂ (0.081 g, 0.23 mmol, 1.5 equiv.) were added into a 4 mL PTFE top screw cap vial. A solution of TPPy (0.070 g, 0.23 mmol, 1.5 equiv.) and BCF (0.015 g, 0.03 mmol, 20 mol %) dissolved in 300 μL of dry DCM, DBM, 1,2-DCE or 1,2-DCB as appropriate was used for step 1 unless otherwise specified. After which, an appropriate source of nucleophile (0.38 mmol, 2.5 equiv.) taken in suitable 300 μL dry solvent was transferred to the reaction mixture. Reaction yields for step 1 and step 2 under different conditions were monitored by ¹⁹F NMR with an internal PhOCF₃ standard and these are summarised in Table 3.

NMR yields for benzotrifluoride scope for S_{N}2 functionalization

| | | | | | | Tem- | | |
|---|---|---|---|---|---|---|---|---|
| En- | | | | | Solvent | pera- | Yield | |
| try | Substrate | Product | Step | Time | (amount) | ture | (%) | Remarks |
| 1 | 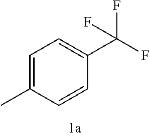 | 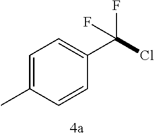 | step 2 | 24 h | DCM (300 μL) | RT | 83 | Nucleophile for step 2: Bn(Et)₃NCl (BTEAC) Also use of (nBu)₄NCl gives 60% yield of 4a; 100% consumption of 3a |
| 2 | 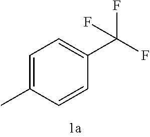 | 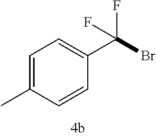 | step 2 | 24 h | DBM (300 μL) | RT | 80 | Nucleophile for step 2: (nBu)₄NBr (TBAB) 100% consumption of 3a; use of DCM gives lower yield |
| 3 | 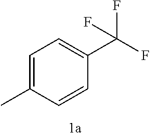 | 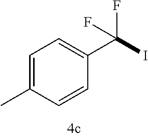 | step 2 | 24 h | DCM (300 μL) | RT | 80 | Nucleophile for step 2: (nBa)₄Nl (TBAl) 100% consumption of 3a |
| 4 | 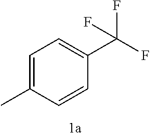 | 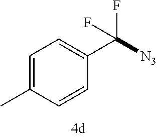 | step 2 | 24 h | DCM (300 μL) | RT | 85 | Nucleophile for step 2: NaN₃ 100% consumption of 3a |
| 5 | 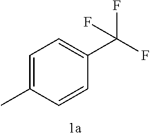 | 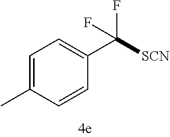 | step 2 | 24 h | DCM (300 μL) | RT | κ-S: 47 κ-N: 37 | Nucleophile for step 2: (nBu)₄NSCN Both products κ-S and κ-N observed; 100% consumption of 3a |
| 6 | 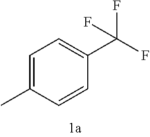 | 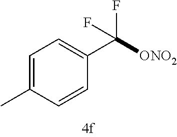 | step 2 | 24 h | DCM (300 μL) | RT | 44 | Nucleophile for step 2: NaNO₃; Due to toxicity of nitrate esters, 4f was not isolated; 100% consumption of 3a |
| 7 | 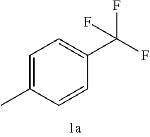 | 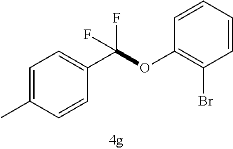 | step 2 | 24 h | DCM (300 μL) | RT | 61 | Nucleophile for step 2: 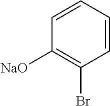 100% consumption of 3a |
| 8 | 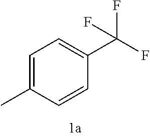 | 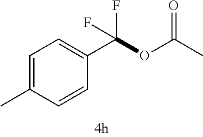 | step 2 | 24 h | DCM (300 μL) | RT | 87 | Nucleophile for step 2 NaOAc Product was prone to hydrolysis during workup and could not be isolated; 100% consumption of 2a |
| 9 | 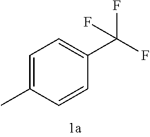 | 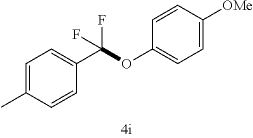 | step 2 | 24 h | DCM (300 μL) | RT | 95 | Nucleophile for step 2: 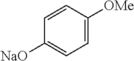 100% consumption of 3a |
| 10 | 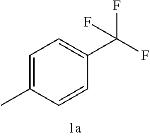 | 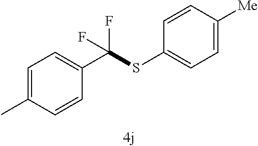 | step 2 | 24 h | DCM (300 μL) | RT | 61 | Nucleophile for step 2: 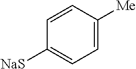 100% consumption of 3a |
| 11 | 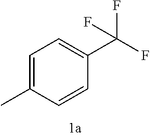 | 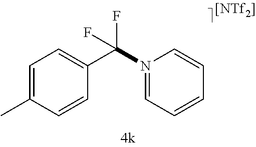 | step 2 | 24 h | DCM (300 μL) | RT | 93 | Nucleophile for step 2: pyridine; 100% consumption of 3a |
| 12 | 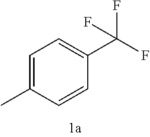 | 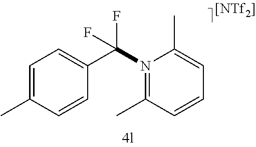 | step 2 | 24 h | DCM (300 μL) | RT | 65 | Nucleophile for step 2: 2,6-lutidine; 100% consumption of 3a |
| 13 | 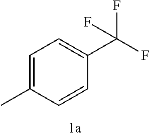 | 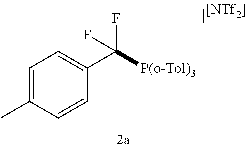 | step 2 | 24 h | DCM (300 μL) | RT | >95 | Nucleophile for step 2: P(o-Tol)₃; 100% consumption of 3a |
| 14 | 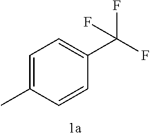 | 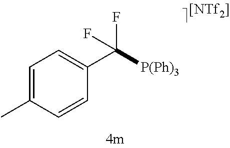 | step 2 | 24 h | DCM (300 μL) | RT | >95 | Nucleophile for step 2: PPh₃; 100% consumption of 3a |
| 15 | 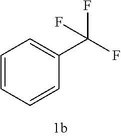 | 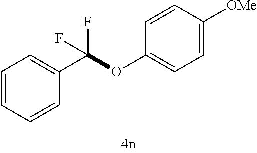 | step 1 step 2 | 18 h 24 h | 1,2-DCE (300 μL) 1,2-DCE (300 μL) | 60° C. RT | >92 58 | Heating at 40° C. in 1,2-DCE for 24 h gives 80% yield 80% consumption of 3b; heated for 24 h at 60° C. during isolation of 4n |
| 16 | 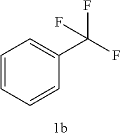 | 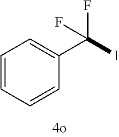 | step 1 step 2 | 18 h 2.5 h | 1,2-DCE (300 μL) 1,2-DCE (600 μL) | 60° C. 60° C. | >92 76 | 100% consumption of 3b |
| 17 | 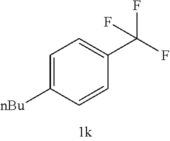 | 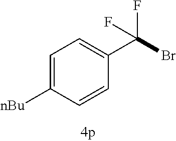 | step 1 step 2 | 48 h 24 h | DBM (300 μL) DBM (300 μL) | RT RT | >95 >95 | 100% consumption of 1k 100% consumption of 3c |
| 18 | 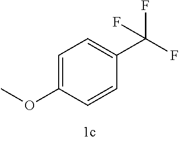 | 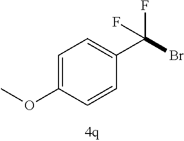 | step 1 step 2 | 15 h 24 h | DCM (600 μL) DCM (300 μL) | RT RT | 50 47 | 100% consumption of 1c; TPPy salt of 1c seems unstable 100% consumption of 3d |
| 20 | 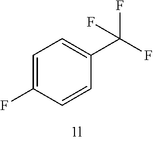 [SMILES: Fc1ccc(C(F)(F)F)cc1] | 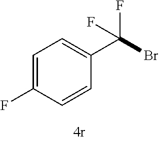 | step 1 step 2 | 48 h 24 h | 1,2-DCE (150 μL) 1,2-DCE (300 μL) | 60° C. RT | 71 65 | 100% consumption of 1e 100% consumption of 3e |
| 21 | 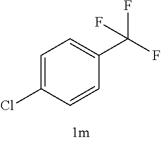 [SMILES: FC(F)(F)c1ccc(Cl)cc1] | 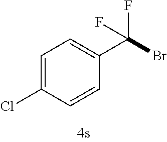 | step 1 step 2 | 48 h 24 h | 1,2-DCE (150 μL) 1,2-DCE (300 μL) | 60° C. RT | 83 76 | 100% consumption of 1m 100% consumption of 3f |
| 22 | 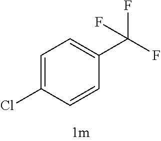 [SMILES: FC(F)(F)c1ccc(Cl)cc1] | 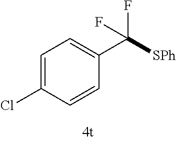 | step 2 | 24 h | 1,2-DCE (300 μL) | RT | 59 | 100% consumption of 3f |
| 23 | 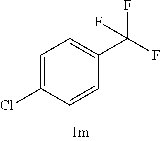 [SMILES: FC(F)(F)c1ccc(Cl)cc1] | 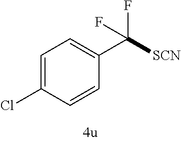 | step 2 | 24 h | 1,2-DCE (300 μL) | RT | κ-S: 29 κ-N: 44 | 100% consumption of 3f |
| 24 | 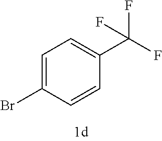 [SMILES: FC(F)(F)c1ccc(Br)cc1] | 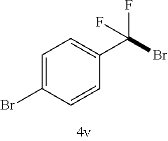 | step 1 step 2 | 48 h 24 h | 1,2-DCE (150 μL) 1,2-DCE (300 μL) | 60° C. RT | 41 41 | 90% consumption of 1g 100% consumption of 3g |
| 25 | 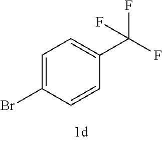 [SMILES: FC(F)(F)c1ccc(Br)cc1] | 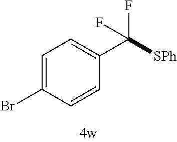 | step 2 | 24 h | 1,2-DCE (300 μL) | RT | 46 | 82% consumption of 3g |
| 26 | 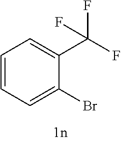 [SMILES: FC(F)(F)c1ccccc1Br] | 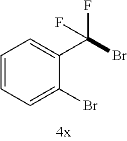 | step 1 step 2 | 3 days 24 h | 1,2-DCE (150 μL) 1,2-DCE (300 μL) | 70° C. 60° C. | — 24 | chemical resonance of TPPy salt overlapped under internal standard 30% consumption of 3h |
| 27 | 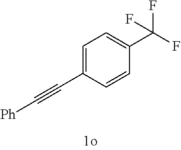 [SMILES: FC(F)(F)c1ccc(C#Cc2ccccc2)cc1] | 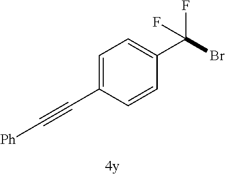 | step 1 step 2 | 4 h 24 h | 1,2-DCE (300 μL) 1,2-DCE (300 μL) | 60° C. RT | 56 56 | 80% consumption of 1o 100% consumption of 3i |
| 28 | 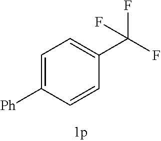 [SMILES: FC(F)(F)c1ccc(-c2ccccc2)cc1] | 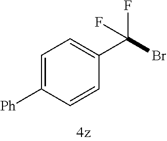 | step 1 step 2 | 48 h 24 h | 1,2-DCE (300 μL) 1,2-DCE (300 μL) | RT RT | 90 83 | 95% consumption of 1p 100% consumption of 3j |
| 29 | 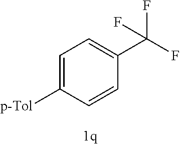 | 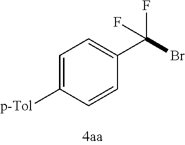 | step 1 step 2 | 48 h 24 h | 1,2-DCE (300 μL) 1,2-DCE (300 μL) | RT RT | >95 32 | 99% consumption of 1q 100% consumption of 3k |
| 30 | 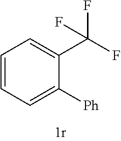 [SMILES: FC(F)(F)c1ccccc1-c1ccccc1] | 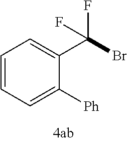 | step 1 step 2 | 48 h 24 h | 1,2-DCE (300 μL) 1,2-DCE (300 μL) | RT 60° C. | >95 18 | 100% consumption of 1r 17% consumption of 3I TPPy salt of 1r seems extremely stable. Due to lower yield, unable to isolate 4ab |
| 31 | 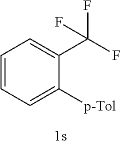 | 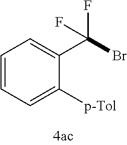 | step 1 step 2 | 48 h 24 h | 1,2-DCE (300 μL) 1,2-DCE (300 μL) | RT 60° C. | >95 20 | 100% consumption of 1s 18% consumption of 3m; TPPy salt of 1s seems extremely stable Due to lower yield, unable to isolate 4ac |
| 32 | 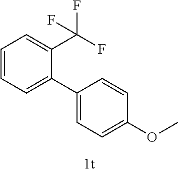 [SMILES: COc1ccc(-c2ccccc2C(F)(F)F)cc1] | 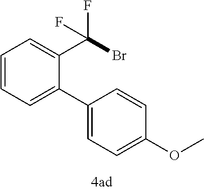 | step 1 step 2 | 48 h 24 h | 1,2-DCE (300 μL) 1,2-DCE (300 μL) | RT 60° C. | >95 12 | 100% consumption of 1t 14% consumption of 3n; TPPy salt of 1t seems extremely stable Due to lower yield, unable to isolate 4ad |
| 33 | 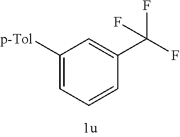 | 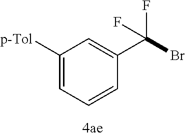 | step 1 step 2 | 48 h 24 h | 1.2-DCE (300 μL) 1,2-DCE (300 μL) | 60° C. RT | 20 20 | 68% consumption of 1u 100% consumption of 3o Due to lower yield, unable to isolate 4ae |
| 34 | 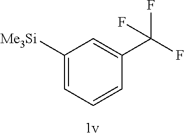 [SMILES: C[Si](C)(C)c1cccc(C(F)(F)F)c1] | 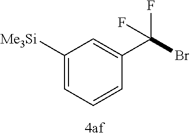 | step 1 step 2 | 48 h 24 h | 1,2-DCE (300 μL) 1,2-DCE (300 μL) | 60° C. RT | 65 63 | 100% consumption of 1v 100% consumption of 3p |
| 35 | 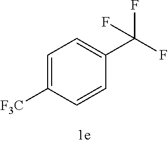 [SMILES: FC(F)(F)c1ccc(C(F)(F)F)cc1] | 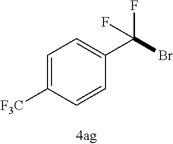 | step 1 step 2 | 3 days 24 h | 1,2-DCE (150 μL) 1,2-DCE (300 μL) | 70° C. 60° C. | 25 14 | 25% consumption of 1q 56% consumption of 3q |
| 36 | 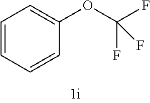 [SMILES: FC(F)(F)Oc1ccccc1] | 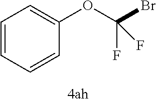 | step 1 step 2 | 60 h 24 h | 1,2-DCB (300 μL) 1,2-DCS (300 μL) | 100° C. 100° C. | 84 84 | 100% consumption of 3r. |
| 37 | 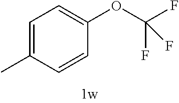 | 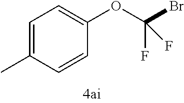 | step 1 step 2 | 48 h 24 h | 1,2-DCB (300 μL) 1,2-DCB (300 μL) | 100° C. 100° C. | — 51 | TBAB added directly after step 1 100% consumption of 3s. |

### Example 4: Large Scale Syntheses and Isolation of Phosphonium Salts 2a-d, 2g-h, 2j

In a 4 mL open PTFE top screw cap vial B(C₆F₅)₃(0.061 g, 0.12 mmol, 20 mol %), Me₃SiNTf₂ (0.325 g, 0.92 mmol, 1.5 equiv.) and P(o-Tol)₃ (0.280 g, 0.92 mmol, 1.5 equiv.) were taken. After addition of 1,2-DCE (1.2 mL) solution of a selected compound from 1a-d, 1g-h, 1j (0.60 mmol. 1.0 equiv.), the reaction mixture was allowed to heat at 80° C. for 24 hours during which the solution turned yellow. Complete reaction was confirmed by ¹⁹F NMR then all volatiles were removed in vacuo. The residue was washed with n-hexane (3×0.5 mL). The resultant solid material was dissolved in DCM and treated with 10% NaHCO₃ (3×0.5 mL), dried over Na₂SO₄ and after removal of all volatiles, the residue was washed again with n-hexane (3×0.5 mL). The resulted sticky material was dissolved in DCM and layered with n-hexane 1:5 (DCM:n-hexane) and stored at 5° C. Crystals appeared after three days and were collected, dried to afford 2. Phosphonium salts were isolated and characterised, and their data are reported below, except for 2e and 2f as both cases we observe a trace of doubly activated products along with 2e/2f giving an inseparable mixture of salts. However, the products are confirmed by ¹⁹F NMR (2e: δ_{F}-64.4 (s, 3 F, p-CF₃), −79.1 (s, 6 F, —CF₃ of —NTf₂), −81.9 (d, J=102.4 Hz, 2 F, Ar—CF₂—P); 2f: δ_{F}-63.7 (s, 3 F, m-CF₃), −78.7 (s, 6 F, —CF₃ of —NTf₂), −82.0 (d, J=103.7 Hz, 2 F, Ar—CF₂—P) and HRMS (ESI-TOF) (m/z: 499.1617 for [C₂₉H₂₅F₅P]⁺ (calcd.: 499.1609).

Compound 2a

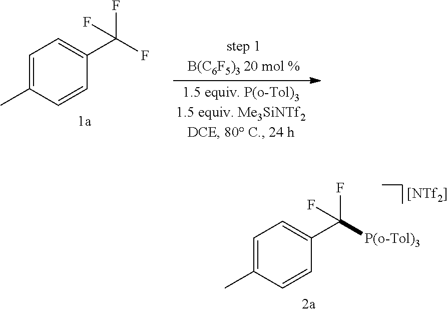

Compound 2a was prepared from 1a (0.877 g, 6.0 mmol, 1 equiv.) based on the protocol above (0.291 g, 67% yield). ¹H NMR (400 MHz, CDCl₃): δ_{H} 7.80 (tt, J=7.8 Hz, 3H, Ar—H of (2—MePh)), 7.78-7.70 (m, 3H, Ar—H of (2—MePh)), 7.60-7.45 (m Ar—H of (2—MePh)), 7.16 (d, J=8.0 Hz, 2H, Ar—H), 7.03 (d, J=8.1 Hz, 2H, Ar—H), 2.37 (s, 3H, Ar—Me), 2.07 (s, 9H, 2—MePh); ¹⁹F NMR (377 MHz, CDCl₃): δ_{F}-78.7 (s, 6 F, —CF₃ of —NTf₂), −79.4 (d, J=109.0 Hz, 2 F, Ar—CF₂—P); ³¹P{¹H} NMR (162 MHz, CDCl₃): δ ₚ 31.8 (t, J=109.0 Hz, 1P, ArCF₂—P); ¹³C{¹H} NMR (101 MHz, CDCl₃): δ_{c} 144.7 (d, J=8.9 Hz), 144.3 (d, J=2.4 Hz), 136.4 (d, J=2.9 Hz), 135.9 (dt, J=10.0 Hz, J=2.6 Hz), 134.7 (d, J=11.8 Hz), 129.8 (s), 127.8 (d, J=13.1 Hz), 126.6 (td, J=6.8 Hz, J=2.2 Hz), 121.3 (td, J=270.6 Hz, J=90.0 Hz), 119.8 (q, J=321.5 Hz), 113.4 (d, J=76 Hz), 22.8 (m), 21.3 (s); HRMS (ESI-TOF) m/z: 445.1864 for [C₂₉H₂₈F₂P]⁺ (calcd.: 445.1891).

Compound 2b

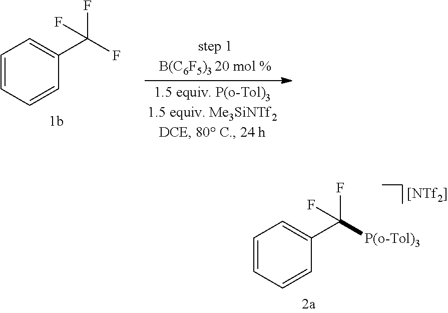

Compound 2b was prepared from 1b (0.877 g, 6.0 mmol, 1 equiv.) based on the protocol above (3.901 g, 91% yield).

1H NMR (400 MHz, CDCl₃): δ_{H} 7.80-7.67 (m, 6H, Ar—f), 7.55-7.47 (m, 7H, Ar—F), 7.34 (t, J=7.7 Hz. 2H, Ar—f), 7.15 (d, J=8.5, 2 H, Ar—f), 2.05 (s, 9H, 2—MePh); ¹⁹F NMR (377 MHz, CDCl₃): δ_{F}-80.3 (d, J=106.9 Hz, 2 F, Ar—CF₂—P), −78.7 (s, 6 F, —CF₃ of —NTf₂); ³¹P{¹H} NMR (162 MHz, CDCl₃): δₚ 32.3 (t, J=106.9 Hz, 1P, ArCF₂—P); ¹³C{¹H} NMR (101 MHz, CDCl₃): 144.69 (d, J=9.2 Hz), 136.42 (d, J=3.3 Hz), 135.87 (dt, J=11.1, 2.6 Hz), 134.70 (d, J=11.9 Hz), 133.19 (d, J=1.9 Hz), 129.75-129.22 (m), 129.20 (s), 127.80 (d, J=12.9 Hz), 126.53 (td, J=6.9, 2.3 Hz), 122.85 (td, J=280.0, 89.5 Hz), 119.84 (q, J=321.8 Hz), 113.29 (d, J=75.9 Hz), 22.8 (m, 3 C, 2—MePh); HRMS (ESI-TOF) m/z: 431.1748 for [C₂₈H₂₆F₂P]⁺ (calcd.: 431.1735).

Compound 2c

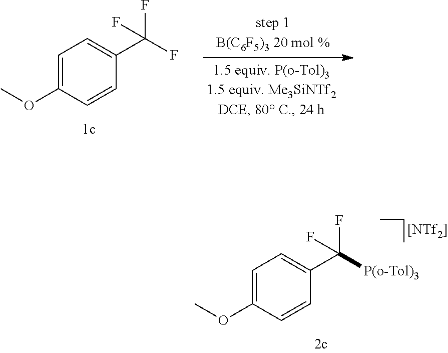

Compound 2c was prepared from 1c (0.104 g, 0.60 mmol. 1.0 equiv.) based on the protocol above except the reaction mixture was heated at 80° C. for 4 hours (0.268 g, 61% yield). ¹H NMR (400 MHz, CD₂Cl₂): δ_{H} 7.91-7.64 (m, 6H, Ar—f), 7.62-7.45 (m, 6H, Ar—H), 7.10 (d, J=9.1 Hz, 2H, Ar—f), 6.85 (d, J=9.2 Hz, 2H, Ar—f), 3.81 (s, 3H, Ar—OMe), 2.08 (s, 9H, 2—MePh); ¹⁹F NMR (377 MHz, CD₂Cl₂): δ_{F}-78.7(d, J=113.2 Hz, 2 F, Ar—CF₂—P), −79.5 (s, 6 F, —CF₃ of—NTf₂); ³¹P{¹H} NMR (162 MHz, CD₂Cl₂): δ_{P} 31.3 (t, J=113.2 Hz, 1P, ArCF₂—P); ¹³C{¹H} NMR (101 MHz, CD₂Cl₂): δ_{c} 163.8 (s), 144.5 (d, J=8.8 Hz), 136.9 (d, J=3.4 Hz), 136.7 (dt, J=11.0 Hz, J=3.0 Hz), 135.3 (d, J=11.7 Hz), 129.2 (td, J=7.2 Hz, J=2.1 Hz), 128.3 (d, J=12.5 Hz), 123.8 (td, J=270.2 Hz, J=90.2 Hz), 120.5 (q, J=320.0 Hz), 115.1 (s), 114.3 (d, J=75.5 Hz), 56.3 (s, 1 C, Ar—OMe), 23.3 (m, 3 C, 2—MePh); HRMS (ESI-TOF) m/z: 461.1840 for [C₂₉H₂₈F₂PO]⁺ (calcd.: 461.1840).

Compound 2d

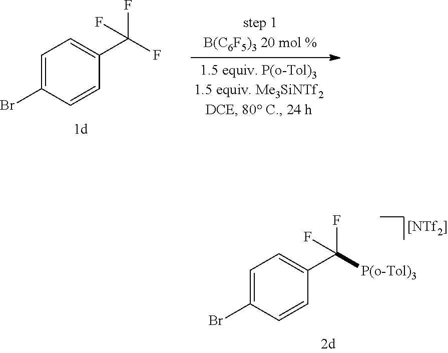

Compound 2d was prepared from 1d (0.135 g, 0.60 mmol. 1.0 equiv.) based on the protocol above (0.419 g, 88% yield). ¹H NMR (400 MHz, CD₂Cl₂): δ_{H} 7.91-7.69 (m, 6H, Ar—f), 7.59-7.52 (m, 8H, Ar—F), 7.08 (d, J=8.1, 2 H, Ar—f), 2.09 (s, 9H, 2—MePh); ¹⁹F NMR (377 MHz, CD₂Cl₂): δ_{F}-81.3 (d, J=105.7 Hz, 2 F, Ar—CF₂—P), −80.0 (s, 6 F, —CF₃ of —NTf₂); ³¹P{¹H} NMR (162 MHz, CD₂Cl₂): δ_{P}31.6 (t, J=105.7 Hz, 1P, ArCF2-P); ¹³C{¹H} NMR (101 MHz, CD₂Cl₂): 145.5 (d, J=9.4 Hz), 137.1 (d, J=3.1 Hz), 136.7 (dt, J=11.2 Hz, J=2.6 Hz), 135.4 (d, J=11.8 Hz), 133.1 (s), 129.3 (td, J=21.9, J=14.5 Hz), 128.9 (td, J=6.8 Hz, J=2.2 Hz), 128.5 (d, J=12.9 Hz), 123.2 (td, J=280.04 Hz, J=90.0 Hz), 120.5 (q, J=321.7 Hz), 113.24 (d, J=76.0 Hz), 23.3 (m, 3 C, 2—MePh); HRMS (ESI-TOF) m/z: 509.0841 for [C₂₈H₂₅BrF₂P]⁺ (calcd.: 509.0840).

Compound 2q

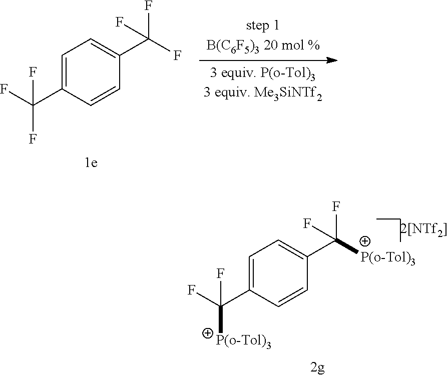

Compound 2g was prepared from 1e (0.129 g, 0.60 mmol. 1.0 equiv.) based on the protocol above except the reaction was performed at 150° C. for 16 hours in 1,2-DCB and this gave orange crystals with oil which was dried and afforded orange powder 2g (0.557 g, 66% yield). ¹H NMR (400 MHz, CDCl₃): δ_{H} 7.86-7.80 (tt, J=7.8, J=2.0 Hz, 6H, Ar—f), 7.74-7.64 (m, 6H, Ar—f), 7.56-7.50 (m, 12H, Ar—f), 7.32 (s, 4H, Ar—f), 2.06 (s, 18H, 2—MePh); 19F NMR (377 MHz, CD₂Cl₂): δ_{F}-80.7 (d, J=101.7 Hz, 2 F, Ar—CF₂—P), −79.4 (s, 12 F, —CF₃ of —NTf₂); ³¹P{¹H} NMR (162 MHz, CD₂Cl₂): δ_{P} 33.8 (t, J=101.7 Hz, 1P, ArCF₂—P); ¹³C{¹H} NMR (101 MHz, CD₂Cl₂): 145.5 (d, J=9.7 Hz), 137.3 (d, J=3.0 Hz), 136.6 (dt, J=11.7 Hz, J=2.2 Hz), 135.5 (d, J=11.9 Hz), 128.7 (d, J=13.1 Hz), 128.4 (t, J=5.90 Hz), 123.7 (td, J=283.7 Hz, J=90.0 Hz), 120.5 (q, J=320.9 Hz), 113.4 (d, J=76.1 Hz), 23.4 (m, 3 C, 2—MePh); HRMS (ESI-TOF) m/z: 392.1504 for [C₅₀H₄₆F₄P₂]⁺ (calcd.: 392.1500).

Compound 2h

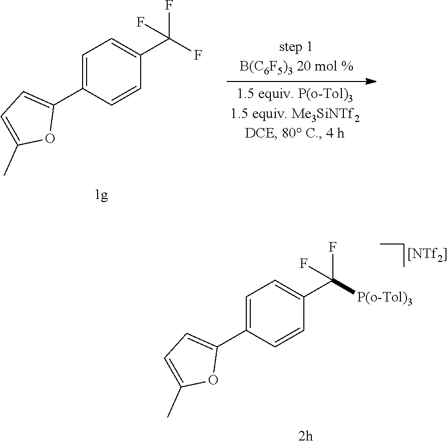

Compound 2h was prepared from 1g based on the protocol above (0.062 g, 52% yield). ¹H NMR (400 MHz, CDCl₃): δ_{H} 7.88-7.69 (m, 7H, Ar—f), 7.60-7.45 (m, 7H, Ar—f), 7.12 (d, J=8.8 Hz, 2H, Ar—F), 6.70 (d, J=3.4 Hz, 1H, Furan-f), 6.13-6.07 (m, 1H, Furan-H), 2.36 (s, 3H. Furan-Me), 2.08 (s, 9H, 2—MePh); ¹⁹F NMR (377 MHz, CDCl₃): δ_{F}-79.6 (d, J=109.2 Hz, 2 F, Ar—CF₂—P), −78.7 (s, 6 F, —CF₃ of —NTf₂); ³¹P{¹H} NMR (162 MHz, CDCl₃): δₚ 31.6 (t, J=109.2 Hz, 1P, ArCF₂—P); ¹³C{¹H} NMR (101 MHz, CDCl₃): δ_{c} 154.3 (s, 1 C, Furan-C), 149.7 (s, 1 C, Furan-C), 144.9 (d, J=9.1 Hz), 136.5 (d, J=2.9 Hz), 136.1 (d, J=11.2 Hz), 134.8 (d, J=11.5 Hz), 133.1 (s), 130.0 (d, J=5.2 Hz), 128.6 (s), 127.9 (d, J=12.5 Hz), 127.2 (t, J=6.6 Hz), 122.6 (td, J=271.0 Hz, J=97.0 Hz), 119.9 (q, J=320.8 Hz), 113.5 (d, J=79.6 Hz), 110.0 (s, 1 C, Furan-C), 108.7 (s, 1 C, Furan-C) 22.8 (m, 3 C, 2—MePh), 13.7 (2—Me-Furan); HRMS (ESI-TOF) m/z: for 511.1999 [C₃₃H₃₀F₂OP]⁺ (calcd.: 511.1997).

Compound 2i

Compound 2i was generated from 1h by following above protocol. The key characterization data is included here from the crude mixture. ¹⁹F NMR (377 MHz, 1,2-DCB): δ_{F},-78.7 (s, 6 F, —CF₃ of—NTf₂) (py—CF₂—P not resolved as chemical shift overlaps with-NTf₂); ³¹P{¹H} NMR (202 MHz, 1,2-DCB): 6p 41.1 (t, J=97.2 Hz, 1P, ArCF₂—P); HRMS (ESI-TOF) m/z: 432.1658 for [C₂₇H₂₅F₂NP]⁺ (calcd.: 432.1687).

Compound 2i

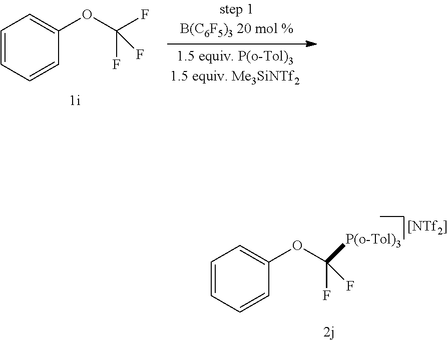

Compound 2j was prepared from 1i based on the protocol above except the reaction mixture was heated at 100° C. for 50 hours in 1,2-DCB. After crystallization, an oil appeared which was dried and afforded 2j (0.280 g, 64% yield). ¹H NMR (400 MHz, CDCl₃) b_{H} 7.92-7.85 (m, 3H, Ar—f), 7.77-7.69 (m, 3H, Ar—F), 7.57-7.49 (m, 3H, Ar—f), 7.45-7.38 (m, 2H, Ar—f), 7.36-7.29 (m, 1H, Ar—f), 7.25-7.17 (m, 3H, Ar—f), 7.13 (d, J=8.2 Hz, 2H), 2.54 (s, 9H, 2—MePh); ¹⁹F NMR (377 MHz, CDCl₃): δ_{F}-53.4 (bs, 2 F, Ar—O—CF₂—P), −78.7 (s, 6 F, —CF₃ of —NTf₂); ³¹P{¹H} NMR (162 MHz, CDCl₃): δₚ 43.7 (bs, 1P, ArCF₂—P); ¹³C{¹H} NMR (101 MHz, CDCl₃): δ_{c} 148.9 (d, J=6.9 Hz), 145.1 (d, J=8.4 Hz), 137.1 (d, J=3.1 Hz), 136.2 (d, J=13.6 Hz), 134.4 (d, J=11.6 Hz), 130.4, 128.4 (d, J=14.0 Hz), 127.7, 120.5, 120.3 (td, J=305.3, 142.5 Hz), 119.9 (q, J=321.8 Hz), 111.6 (d, J=81.5 Hz), 23.1 (q, J=3.3 Hz); HRMS (ESI-TOF) m/z: 447.1667 for [C₂₈H₂₆F₂OP]⁺ (calcd.: 447.1684).

Compound 2k

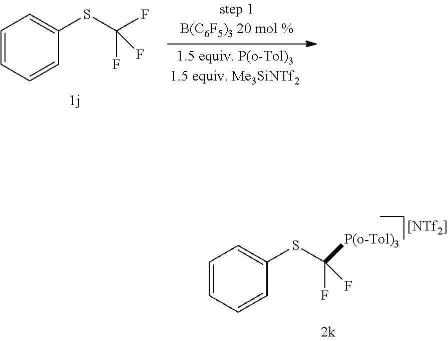

Compound 2k was prepared from 1j based on the protocol above except the reaction mixture heated at 100° C. for 50 hours in 1,2-DCB. After crystallization an oil appeared, dried and afforded 2k (0.320 g, 72% yield). ¹H NMR (400 MHz, CD₂Cl₂) b_{H} 8.01-7.80 (m, 3H, Ar—f), 7.76-7.46 (m, 14H, Ar—f), 2.40 (s, 9H, 2—MePh); ¹⁹F NMR (377 MHz, CD₂Cl₂): δ_{F}-61.8(bs, 2 F, Ar—O—CF₂—P), −79.3 (s, 6 F, —CF₃ of —NTf₂); ³¹P{¹H} NMR (162 MHz, CD₂Cl₂): δ_{P} 40.7 (bs, 1P, ArCF₂—P); ¹³C{¹H} NMR (101 MHz, CDCl₃): δ_{c} 146.1 (d, J=8.9 Hz), 137.6 (d, J=3.0 Hz), 137.4 (s), 137.0 (d, J=12.8 Hz), 135.1 (d, J=11.9 Hz), 132.6 (s), 130.6 (s), 128.9 (d, J=14.0 Hz), 122 120.5 (q, J=319.2 Hz), 112.8 (d, J=75.6 Hz), 24.1 (m, 3 C, 2—MePh) (Ph-S—CF₂—P not resolved); HRMS (ESI-TOF) m/z: 463.1474 for [C₂₈H₂₆F₂OP]⁺ (calcd.: 463.1455).

### Example 5: General Method for Large Scale Syntheses and Isolation of TPPy Salts 3a-3b, 31-3n

B(C₆F₅)₃(0.061 g, 0.12 mmol, 20 mol %), trifluoride selected from 1a-1b, 1r-1t (0.60 mmol. 1.0 equiv.), and Me₃SiNTf₂ (0.318 g, 0.90 mmol, 1.5 equiv.) were dissolved in dry DCM (0.6 mL). After addition of DCM (0.6 mL) solution of TPPy (0.277 g, 0.90 mmol, 1.5 equiv.), the reaction mixture was allowed to stir at RT for 48 hours for 3a and at 60° C. for 18 hours for 3b, 31-3n during which the brown reaction solution had changed to yellowish-brown. After complete reaction was confirmed by ¹⁹F NMR analysis of the crude reaction mixture, all volatiles were removed in vacuo. The residue was washed with dry toluene (3×5 mL) and further dried in vacuo. The resultant yellow solid material was dissolved in DCM and treated with 10% NaHCO₃ (3×5 mL). Followed by drying over Na₂SO₄ and removal of all volatiles, the residue was washed again with toluene (3×5 mL). The resultant yellow solid was dissolved in DCM and layered with n-hexane 1:5 (DCM:n-hexane) and stored at 5° C. Faint yellow crystals appeared after two days, which were collected and dried to yield the desired TPPy salts. The exact conditions and characterising information for the resulting compounds are provided below.

The procedure above may be replaced by analogy by procedure B in Example 3. The use of this modified procedure may result in greater yields of the desired product.

Compound 3a

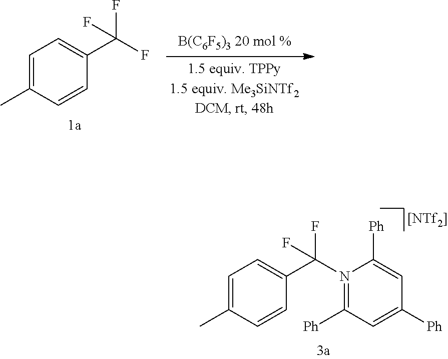

3a was prepared from 1a based on the protocol above. Due to limited stability of 3a, characterization was performed on the crude residue and key characterization data are included here.

¹⁹F NMR (377 MHz, CDCl₃): δ_{F}-55.0 (s, ArCF₂-TPPy), −78.4 (s, —CF₃ of —NTf₂); ¹³C{¹H} NMR (126 MHz, CDCl₃): δ_{c} 122.3 (t, J=273.0 Hz, ArCF₂-TPPy); HRMS (ESI-TOF) m/z: 448.1842 for [C₃₁H₂₄F₂N]⁺ (calcd.: 448.1871).

Compound 3b

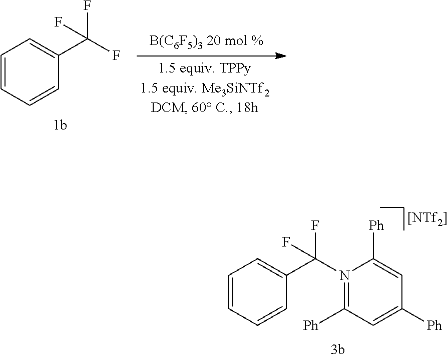

3b was prepared from 1b based on the protocol above. Faint yellow crystals appeared after two days, which were collected and dried to yield 3b (0.228 g, 53% yield). ¹H NMR (400 MHz, CDCl₃): δ_{H} 8.03 (s, 2H, —C₅H₂N), 8.01-7.95 (m, 2H, Ar—H), 7.67-7.50 (m, 9H, Ar—H), 7.50-7.42 (m, 4H, Ar—H), 7.30 (t, J=7.8 Hz, 1H, Ar—H), 7.15 (t, J=8.2 Hz, 2H, Ar—H), 6.87 (d, J=7.4 Hz, 2H, Ar—H); ¹⁹F NMR (377 MHz, CDCl₃): δ_{F}-55.3(s, 2 F, ArCF₂-TPPy), −78.7 (s, 6 F, —CF₃ of —NTf₂); ¹³C{¹H} NMR (126 MHz, CDCl₃): δ_{c} 159.1 (s), 158.6 (s), 135.0 (s), 133.9 (s), 132.9 (s), 132.7 (s), 132.1 (s), 131.7 (s), 131.6 (s), 130.1-130.0 (m), 129.1 (s), 129.0 (d, J=6.3 Hz), 128.5 (s), 127.9 (s), 127.7 (s), 125.2 (t, J=3.7 Hz), 121.1 (t, J=270.6 Hz), 120.0 (q, J=320.0 Hz); HRMS (ESI-TOF) m/z: 434.1685 for [C₃₀H₂₂F₂N]⁺ (calcd.: 434.1715).

Compound 3I

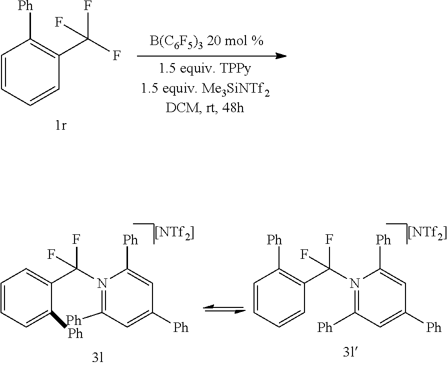

3I was prepared from 1r based on the protocol above except the reaction mixture stirred at RT for 48 hours and afforded yellow crystals which were mixture of 31 and 31′ (total yield: 0.277 g, 58%). An approximate ratio of 31:31′ is 97:3 based on ¹⁹F NMR. Due to two components, only key chemical resonances are listed here. ¹H NMR (400 MHz, CDCl₃): δ_{H} 8.10 (s,-C5H₂N), 7.81-7.71 (m, Ar—H), 7.64-7.58 (m, Ar—H), 7.39-7.30 (m, Ar—H), 7.09 (d, J=8.8 Hz, Ar—H), 6.96 (d, J=7.0 Hz, Ar—H), 6.90 (t, J=8.0 Hz, Ar—H), 6.82 (d, J=6.8 Hz, Ar—H); ¹⁹F NMR (377 MHz, CDCl₃): δ_{F}-44.7 (d, J=188.3 Hz, 1 F, ArCF₂-TPPy for 31 form), −55.9 (d, J=188.3 Hz, 1 F, ArCF₂-TPPy for 31 form), −56.9 (s, 2 F, ArCF₂-TPPy for 31′ form), −78.7 (s, 6 F, —CF₃ of —NTf₂); ¹³C{¹H} NMR (101 MHz, CDCl₃): δ_{c} 158.6 (s), 157.9 (s), 138.8 (s), 137.4 (s), 133.9 (s), 133.0 (s), 132.3 (s), 131.8 (d, J=5.8 Hz), 130.1 (s), 129.8 (s), 128.9 (s), 128.4 (d, J=7.3 Hz), 127.2 (s), 126.6 (t, J=6.4 Hz), 121.4 (t, J=276.0 Hz), 120.0 (q, J=321.7 Hz). HRMS (ESI-TOF) m/z: 511.4812 for [C₃₆H₂₆F₂N]⁺ (calcd.: 510.2027).

Compound 3m

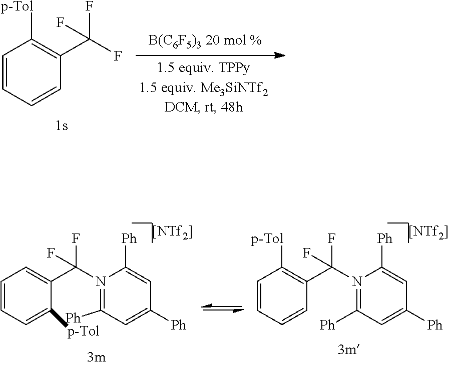

3m was prepared from 1s based on the protocol above except the reaction mixture was stirred at RT for 48 hours and afforded yellow crystals which were dried and afforded a mixture of 3m and 3m′ (total yield: 0.298 g, 62%). An approximate ratio of 3m:3m′ is 91:9 based on ¹⁹F NMR analysis. Due to two isomers, key chemical resonances are included. ¹H NMR (400 MHz, CDCl₃): δ_{H} 8.13 (d, J=1.6 Hz, —C₅H₂N), 7.83-7.79 (m, Ar—H), 7.65-7.60 (m, Ar—H), 7.34 (t, J=7.3 Hz, Ar—H), 7.1 (d, J=7.3 Hz, Ar—H), 6.90-6.86 (m, Ar—H), 6.83 (dd, J=8.0 Hz, J=1.0 Hz, Ar—H), 2.41 (s, (p-Tol)—Me for 3m′), 2.24 (s, (p-Tol)—Me for 3m); ¹⁹F NMR (377 MHz, CDCl₃): δ_{F}-43.9(d, J=194.3 Hz, 1 F, ArCF₂-TPPy for 3m form), −55.6 (d, J=194.3 Hz, 1 F, ArCF₂-TPPy for 3m form), −56.9 (s, 2 F, ArCF₂-TPPy for 3m′ form), −78.7 (s, 6 F, —CF₃ of —NTf₂); ¹³C{¹H} NMR (101 MHz, CDCl₃): δ_{c} 158.3 (s), 157.9 (s), 154.1 (s), 138.3 (s) 134.5 (s), 134.0 (s), 133.2 (s), 132.7 (s), 132.1 (s), 131.9 (d, J=4.8 Hz), 129.9 (d, J=7.4 Hz), 129.4 (br s), 128.8 (s), 128.4 (d, J=8.0 Hz), 128.0 (s), 127.1 (s), 126.6 (t, J=6.2 Hz), 121.3 (t, J=275.0 Hz), 120.1 (q, J=322.6 Hz), 21.2 (s, 1 C, Ar—Me for 3m′), 20.9 (s, 1 C, Ar—Me for 3m); HRMS (ESI-TOF) m/z: 525.5255 for [C₃₇H₂₈F₂N]⁺ (calcd.: 524.2184).

Compound 3n

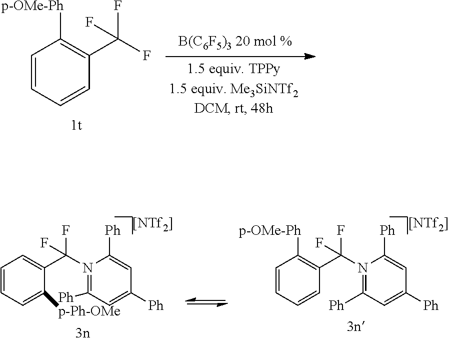

3n was prepared from it by following similar protocol described in example 15 except the reaction mixture was stirred at RT for 48 hours. Following purification, an oil was collected, dried and afforded the isomers 3n/3n′ (total yield: 0.224 g, 45%).

Spectroscopic data could not be resolved for 3n and 3n′ due to fast exchange on the NMR time scale. Thus, the ¹H NMR data is based on the mixture of 3n and 3n′: ¹H NMR (400 MHz, CDCl₃): δ_{H} 7.91 (s, 2H, —C₅H₂N), 7.78-7.72 (m, 3H, Ar—H), 7.70-7.65 (m, 5H, Ar—H), 7.61-7.55 (m, 8H, Ar—H), 7.55 (t, J=1.5 Hz, 1H, Ar—H), 7.54-7.52 (m, 1H, Ar—H), 7.52-7.50 (m, 1H, Ar—F), 7.49 (t, J=1.7 Hz, 1H), 3.82 (s, 3H,-OMe); ¹⁹F NMR (377 MHz, CDCl₃): δ_{F}-56.6 (brs, 2 F, ArCF2-TPPy), −78.7 (s, 6 F, —CF₃ of—NTf₂); ¹³C{¹H} NMR (101 MHz, CDCl₃): δ_{c} 158.3 (s), 157.1 (s), 156.2 (s), 133.8 (s), 132.3 (d, J=9.5 Hz), 131.5 (s), 129.7 (s), 129.4 (s), 128.9 (s), 127.9 (s), 125.9 (s), (t, J=276.1 Hz), 119.8 (q, J=323.0 Hz), 45.0 (s); HRMS (ESI-TOF) m/z: 525.5255 for [C₃₇H₂₈F₂N]⁺ (calcd.: 524.2184).; HRMS (ESI-TOF) m/z: 541.5553 for [C₃₇H₂₈F₂NO]⁺ (calcd.: 540.2133).

### Example 6: General Method for One-Pot Syntheses of Difluorinated Compounds Via TPPy Salt

Procedure A

In a 20 mL screw cap with septa vial, B(C₆F₅)₃(0.061 g, 0.12 mmol, 20 mol %), trifluorides selected from compound 1a-b, 1o, 1q, lv, Fluoxetine (0.60 mmol. 1.0 equiv.) and Me₃SiNTf₂ (0.318 g, 0.90 mmol, 1.5 equiv.) were dissolved in dry DCM (0.6 mL). After addition of DCM (0.6 mL) solution of TPPy (0.277 g, 0.90 mmol, 1.5 equiv.), the reaction mixture was allowed to stir at RT for 48 hours during which the brown reaction solution turned yellowish-brown. In step 2, a DCM solution (0.6 mL) of nucleophile (1.50 mmol, 2.5 equiv.) was transferred to the reaction mixture and left at RT for 24 hours. After removal of all volatiles, column chromatography purification of the crude residue afforded 4.

The yields below are based upon the use of procedure A.

Procedure B

It was discovered that by changing the order to adding the reagents, it was possible to improve the reaction yields obtained by up to 10-20%.

In a 20 mL screw cap with septa vial, trifluorides selected from compound 1a-b, 1o, 1q, iv, Fluoxetine (0.60 mmol. 1.0 equiv.) and Me₃SiNTf₂ (0.318 g, 0.90 mmol, 1.5 equiv.) were dissolved in dry DCM (0.6 mL). After addition of DCM (0.6 mL) solution of TPPy (0.277 g, 0.90 mmol, 1.5 equiv.) and B(C₆F₅)₃(0.061 g, 0.12 mmol, 20 mol %), the reaction mixture was allowed to stir at RT for 48 hours during which the brown reaction solution turned yellowish-brown. In step 2, a DCM solution (0.6 mL) of nucleophile (1.50 mmol, 2.5 equiv.) was transferred to the reaction mixture and left at RT for 24 hours. After removal of all volatiles, column chromatography purification of the crude residue afforded 4. The exact conditions used for each reaction are detailed below in conjunction with the characterising data for each product.

Compound 4a

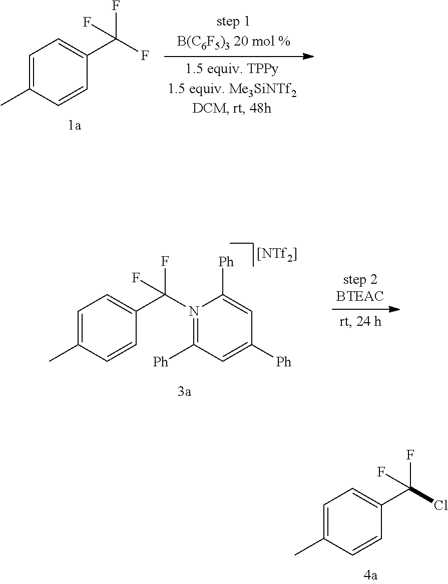

Compound 4a was prepared from 1a based on the protocol above where BTEAC (0.341 g, 1.50 mmol, 2.5 equiv.) was added in step 2. Column chromatography purification using an eluent system of n-pentane afforded colourless oil 4a (0.055 g, 52% yield). ¹H NMR (400 MHz, CDCl₃): δ_{H} 7.51 (d, J=8.1 Hz, 2H), 7.27-7.23 (m, 2H), 2.40 (s, 3H);19F NMR (377 MHz, CDCl₃): δ_{F}-47.8 (s, 2 F); ¹³C{¹H} NMR (126 MHz, CDCl₃): δ_{c} 141.7 (t, J=1.5 Hz, 2 C), 133.7 (t, J=26.3 Hz, 1 C), 129.2 (s, 1 C), 126.8 (t, J=289.1 Hz, 1 C), 124.6 (t, J=4.9 Hz, 2 C), 21.3 (s, 1 C). HRMS (APCI) m/z: 176.0200 for [C₈H₇ClF₂]⁺ (calcd.: 176.0199).

Compound 4b

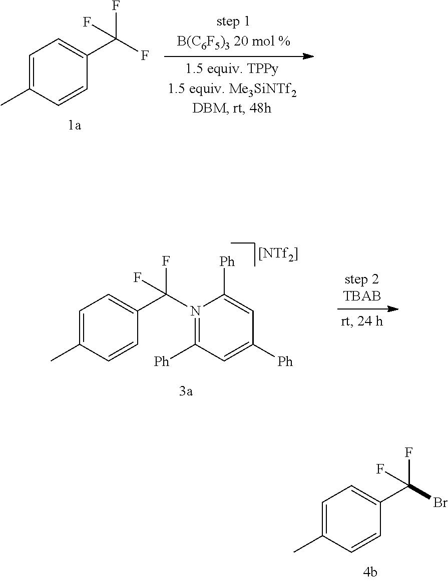

Compound 4b was prepared from 1a based on the protocol above except DBM was used as the solvent instead of DCM. TBAB (0.484 g, 1.50 mmol, 2.5 equiv.) dissolved in DBM was added in step 2 and column chromatography purification with an eluent n-hexane afforded colourless oil 4b (0.055 g, 42% yield). ¹H NMR (400 MHz, CDCl₃): δ_{H} 7.51 (d, J=8.0 Hz, 2H), 7.26 (d, J=8.0 Hz, 2H), 2.41 (s, 3H); ¹⁹F NMR (377 MHz, CDCl₃): δ_{F}-42.55 (s, 2 F); ¹³C{¹H} NMR (126 MHz, CDCl₃): δ_{c} 141.6 (t, J=1.2 Hz, 2 C), 135.5 (t, J=23.6 Hz, 1 C), 129.2 (s, 1 C), 124.3 (t, J=5.1 Hz, 2 C), 118.6 (t, J=303.6 Hz, 1 C), 21.4 (s, 1 C); HRMS (APCI) m/z: 218.9613 for [C₈H₇BrF₂]⁺ (calcd.: 218.9615).

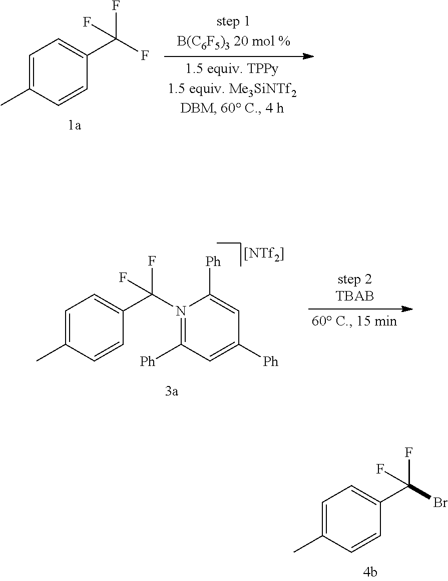

Compound 4b was prepared from 1a in a 4 mL open PTFE top screw cap vial based on the protocol above except that the solution was heated at 60° C. for 4 hours in step 1 and TBAB (0.484 g, 1.50 mmol, 2.5 equiv.) was added in step 2 and heated at 60° C. for a further 15 minutes. PhOCF₃ internal standard (1.0 equiv.) was added and the solution was transferred to an NMR tube for analysis. ¹⁹F NMR spectroscopy revealed a final yield of 80% for 4b.

Compound 4c

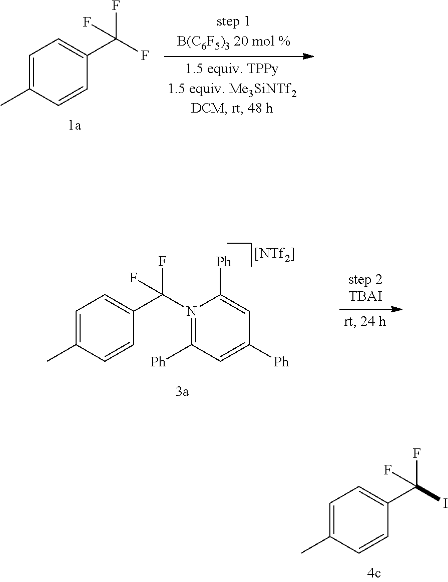

Compound 4c was prepared from 1a based on the protocol above where TBAI (0.554 g, 1.50 mmol, 2.5 equiv.) was added in step 2. Column chromatography purification with an eluent n-hexane gave pink oil 4c (0.010 g, 10%). Decomposition over time leads to lower yield and hence partial characterization included here. ¹H NMR (400 MHz, CDCl₃): δ_{H} 7.46 (d, J=8.1 Hz, 2H), 7.23 (d, J=8.8 Hz, 2H), 2.39 (s, 3H); ¹⁹F NMR (377 MHz, CDCl₃): δ_{F}-35.6 (s, 2 F).

Compound 4d

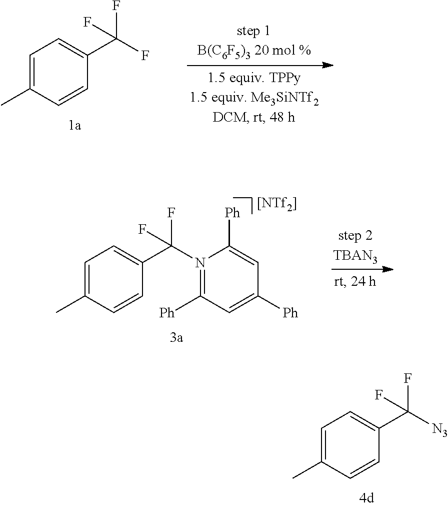

Compound 4d was prepared from 1a based on the protocol above where TBAN₃ (0.427 g, 1.50 mmol, 2.5 equiv.) was added in step 2. Column chromatography purification with an eluent n-hexane gave colourless oil 4d (0.051 g, 46% yield). ¹H NMR (400 MHz, CDCl₃): δ_{H} 7.50 (d, J=8.1 Hz, 2H), 7.29-7.24 (m, 2H), 2.40 (s, 3H); ¹⁹F NMR (377 MHz, CDCl₃): δ_{F}-67.7(s, 2 F); ¹³C{¹H} NMR (126 MHz, CDCl_{3): δc} 141.6 (t, J=1.2 Hz, 2 C), 129.7 (t, J=29.1 Hz, 1 C), 129.3 (s, 1 C), 125.2 (t, J=4.1 Hz, 2 C), 121.7 (t, J=259.6 Hz, 1 C), 21.3 (s, 1 C); HRMS (APCI) m/z: 183.0599 for [CaH₇N3F₂] (calcd.: 183.0603).

Compound 4e

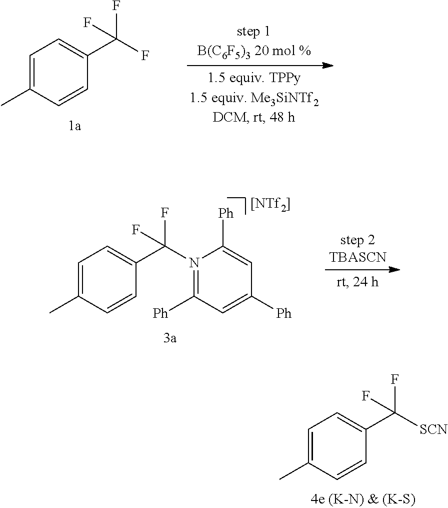

Compound 4e was prepared from 1a based on the protocol above where TBASCN (0.450 g, 1.50 mmol, 2.5 equiv.) was added in step 2. Column purification with an eluent n-hexane afforded both 4e (κ-N) (0.062 g, 52% yield) and 4e (κ-S) (0.005 g, 4% yield). Decomposition of 4e (κ-S) over time results a lower yield. Therefore, partial characterization 4e (κ-S) included here. 4e (κ-N): ¹H NMR (400 MHz, CDCl₃): δ_{H} 7.52 (d, J=8.0 Hz, 2H), 7.29 (d, J=7.9 Hz, 2H), 2.43 (s, 3H); ¹⁹F NMR (377 MHz, CDCl₃): δ_{F}-62.3 (s, 2 F); ¹³C{¹H} NMR (126 MHz, CDCl₃): δ_{c} 141.7 (t, J=1.6 Hz, 2 C), 131.7 (t, J=31.0 Hz, 1 C), 129.4 (s, 1 C), 124.6 (t, J=4.2 Hz, 2 C), 116.5 (t, J=252.1 Hz, 1 C), 21.3 (s, 1 C); HRMS (APCI) m/z: 199.0260 for [C₃H₇N₃F₂]⁺ (calcd.: 199.0262). 4e (κ-S): ¹⁹F NMR (377 MHz, CDCl₃): δ_{F}-64.3 (s, 2 F).

Compound 4q

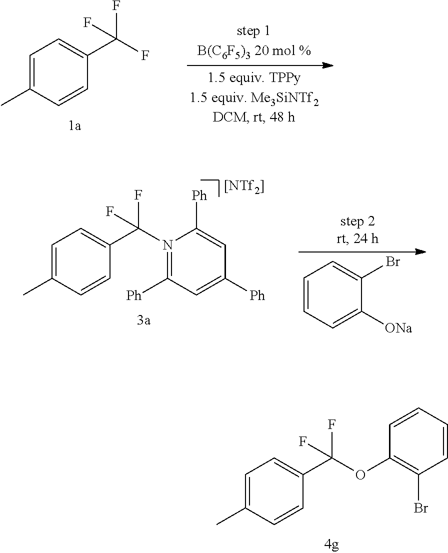

Compound 4g was prepared from 1a based on the protocol above where sodium 2-bromo-phenolate. (0.293 g, 1.50 mmol, 2.5 equiv.) was added in step 2. Column purification using an eluent n-hexane/ethylacetate (99:1) gave colourless oil 4g (0.079 g, 43% yield). ¹H NMR (400 MHz, CDCl₃): δ_{H} 7.74 (d, J=8.0 Hz, 2H), 7.62 (dd, J=8.0, 1.6 Hz, 1H), 7.48 (dq, J=8.2, 1.5 Hz, 1H), 7.35-7.27 (m, 3H), 7.10 (ddd, J=8.0, 7.4, 1.5 Hz, 1H), 2.42 (s, 3H); ¹⁹F NMR (377 MHz, CDCl₃): δ_{F}-64.2 (s, 2 F); ¹³C{¹H} NMR (126 MHz, CDCl_{3): δc} 147.9 (s), 141.2 (t, J=1.4 Hz), 133.6 (s), 130.5 (t, J=31.1 Hz), 129.1 (s), 128.3 (s), 126.7 (s), 125.7 (t, J=3.8 Hz), 123.3 (t, J=1.9 Hz), 122.7 (t, J=263.7 Hz, 1 C), 116.7 (s), 21.4 (s, 1 C); HRMS (APCI) m/z: 292.9973 for [M−F]⁺ (calculated 292.9972 for [C₁₄H₁₁BrFO]⁺).

Compound 4i

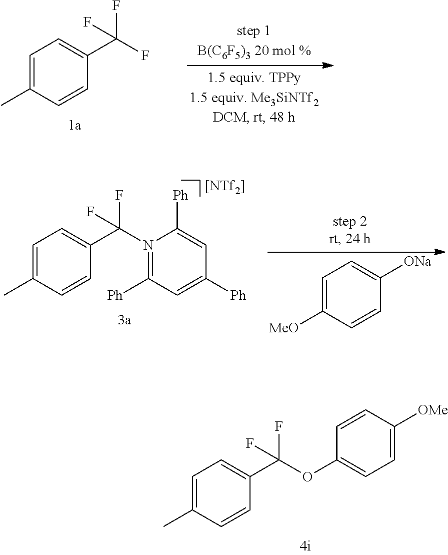

Compound 4i was prepared from 1a based on the protocol above where sodium 4-methoxyphenolate (0.219 g, 1.50 mmol, 2.5 equiv.) was added in step 2. Column purification using an eluent n-hexane/ethyl acetate (99:1) gave colourless oil 4i (0.080 g, 50% yield). ¹H NMR (500 MHz, CDCl₃): δ_{H} 7.63 (d, J=8.0 Hz, 2H), 7.29 (d, J=7.7 Hz, 2H), 7.24-7.17 (m, 2H), 6.94-6.83 (m, 2H), 3.82 (s, 3H), 2.43 (s, 3H); ¹⁹F NMR (377 MHz, CDCl₃): δ_{F}-65.2 (s, 2 F); ¹³C{¹H} NMR (126 MHz, CDCl₃): δ_{c} 157.2 (s), 143.9 (t, J=1.9 Hz, 2 C), 140.7 (s), 131.1 (t, J=32.0 Hz), 129.0 (s), 125.5 (t, J=3.8 Hz), 123.3 (s), 122.4 (t, J=260.3 Hz, 1 C), 114.3 (s), 55.5 (s, 1 C), 21.3 (s, 1 C); HRMS (APCI) m/z: 264.0965 for [C₁₅H₁₄O₂F₂]⁺ (calcd.: 264.0956).

Compound 4i

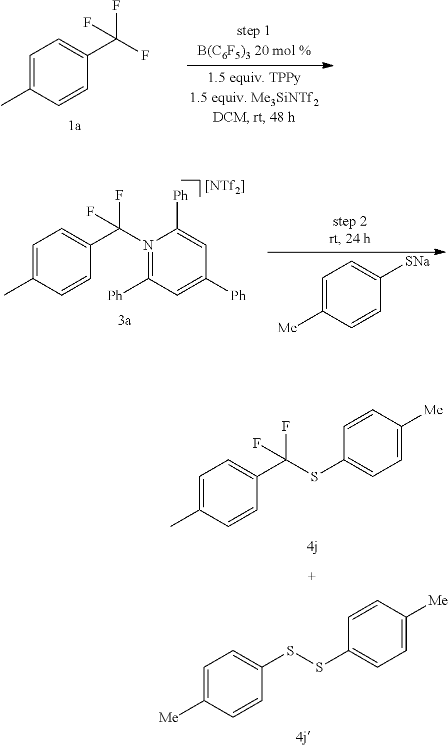

Compound 4j was prepared from 1a based on the protocol above where sodium 4-methylthiophenol (0.079 g, 0.54 mmol, 0.9 equiv.) was used for step 2. Column purification using an eluent n-hexane afforded off-white solid 4j (0.015 g, 10% yield) with oxidised impurity 4j′. Hence partial characterization included here. 4j: ¹H NMR (500 MHz, CDCl₃): δ_{H} 7.53 (d, J=8.1 Hz, 2H), 7.49 (d, J=8.0 Hz, 2H), 7.23 (d, J=8.0 Hz, 2H), 7.20 (d, J=7.7 Hz, 2H), 2.40 (s, 3H), 2.39 (s, 3H); ¹⁹F NMR (377 MHz, CDCl₃): δ_{F}-71.3(s, 2 F).

Compound 4n

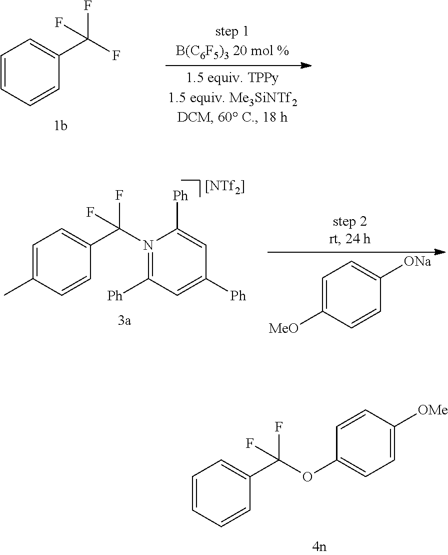

Compound 4n was prepared from 1b based on the protocol above where 4-methoxyphenolate (0.219 g, 1.50 mmol, 2.5 equiv.) was added except the reaction vial was heated at 60° C. for 18 hours in step 1 and heated at 60° C. for 24 hours in step 2. Column purification performed using an eluent n-hexane gave off-white solid 4n (0.116 g, 77% yield). ¹H NMR (400 MHz, CDCl₃): δ_{H} 7.78-7.71 (m, 2H, Ar—f), 7.55-7.44 (m, 3H, Ar—F), 7.23-7.17 (m, 2H, Ar—H), 6.89 (dt, J=9.2 Hz, J=3.3 Hz, 2H, Ar—f), 3.82 (s, 3H, Ar—OMe); ¹⁹F NMR (377 MHz, CDCl₃): δ_{F}-65.7 (s, 2 F, Ar—CF₂—OAr); ¹³C{¹H} NMR (126 MHz, CDCl₃): δ_{C}157.3 (s), 143.9 (s), 133.9 (t, J=32.0 Hz), 130.7 (s), 128.4 (s), 125.6 (t, J=3.80 Hz), 123.3 (s), 122.2 (t, J=260.3 Hz), 114.3 (s), 55.6 (s); HRMS (APCI) m/z: 231.0809 for [M−F]⁺ (calcd. 231.0816 for C₁₄H₁₂02F).

Compound 4y

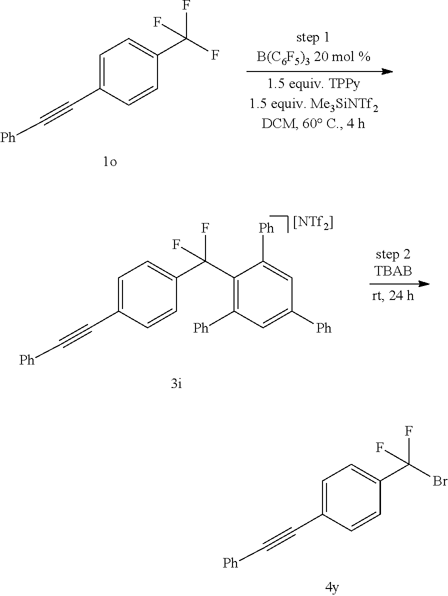 [SMILES: FC(F)(F)c1ccc(C#Cc2ccccc2)cc1 ; FC(F)(c1ccc(C#Cc2ccccc2)cc1)c1c(-c2ccccc2)cc(-c2ccccc2)cc1-c1ccccc1 ; FC(F)(Br)c1ccc(C#Cc2ccccc2)cc1]

Compound 4y was prepared from 10 based on the protocol above except 1,2-DCE was used as the solvent instead of DCM. TBAB (0.484 g, 1.50 mmol, 2.5 equiv.) dissolved in 1,2-DCE was added in step 2 and column chromatography purification with an eluent n-hexane resulted white solid 4y (0.115 g, 62% yield). ¹H NMR (400 MHz, CDCl₃): δ_{H} 7.61 (s, 4H, Ar—f), 7.58-7.53 (m, 2H, Ar—f), 7.41-7.36 (m, 3H, Ar—f); ¹⁹F NMR (377 MHz, CDCl₃): δ_{F}-44.0 (s, 2 F, Ar—CF₂Br); ¹³C{¹H} NMR (126 MHz, CDCl₃): 137.4 (t, J=23.1 Hz), 131.7 (d, J=3.2 Hz), 128.8 (s), 128.4 (s), 126.6 (s), 124.4 (t, J=5.1 Hz), 122.6 (s), 118.0 (t, J=302.5 Hz, 1 C, Ar—CF₂Br), 91.7 (s, 1 C, ethynyl-C), 88.1 (s, 1 C, ethynyl-C); HRMS (APCI) m/z: 306.9938 for [M+H]⁺ (calcd. 306.9928 for C₁₅H₁₀BrF₂).

Compound 4aa

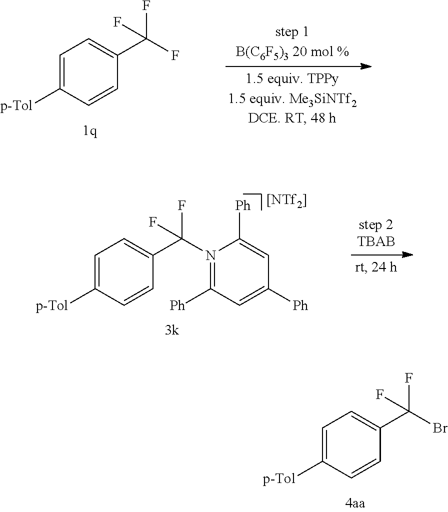

Compound 4aa was prepared from 1q based on the protocol above except 1,2-DCE was used as the solvent instead of DCM. TBAB (0.484 g, 1.50 mmol, 2.5 equiv.) dissolved in 1,2-DCE was added in step 2 and column chromatography purification with an eluent n-hexane gave off-white solid 4aa (0.153 g, 86% yield). ¹H NMR (400 MHz, CDCl₃): δ_{H} 7.74-7.61 (m, 2H, Ar—f), 7.56-7.47 (m, 2H, Ar—f), 7.35-7.28 (m, 2H, Ar—f), 2.43 (s, Ar—Me); ¹⁹F NMR (377 MHz, CDCl₃): δ_{F}-43.6 (s, 2 F, Ar—CF₂Br); ¹³C{¹H} NMR (126 MHz, CDCl₃): δ_{c} 144.2 (s), 138.2 (s), 136.8 (s), 136.6 (t, J=23.3 Hz), 129.7 (s), 127.1 (s), 124.8 (t, J=5.3 Hz), 118.5 (t, J=308.0 Hz, 1 C, Ar—CF₂Br), 21.1 (s); HRMS (APCI) m/z: 217.0821 for [M-Br]⁺ (calcd. 217.0823 for C₁₄H₁₁F₂).

Compound 4af

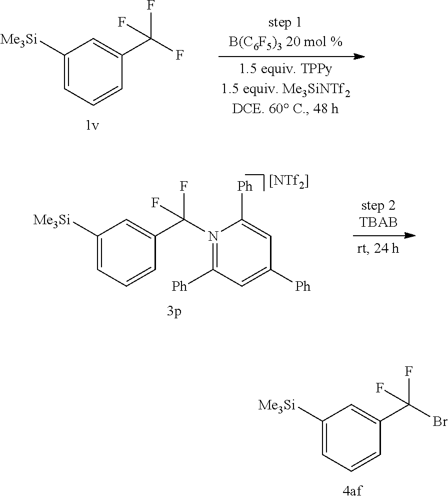

Compound 4af was prepared from 1v based on the protocol above except 1,2-DCE was used as the solvent instead of DCM and the reaction vial was heated at 60° C. for 48 hours. TBAB (0.484 g, 1.50 mmol, 2.5 equiv.) dissolved in 1,2-DCE was added in step 2 and column chromatography purification with an eluent n-hexane gave colourless oil 4af (0.071 g, 42% yield). ¹H NMR (400 MHz, CDCl₃): δ_{H} 7.73 (s, 1H, Ar—f), 7.64 (d, J=7.6 Hz, 1H, Ar—f), 7.60 (d, J=7.9 Hz, 1H, Ar—f), 7.45 (t, J=7.6 Hz, 1H, Ar—f), 0.31 (s, 9H, TMS-F); ¹⁹F NMR (377 MHz, CDCl₃): δ_{F}-43.3(s, 2 F); ¹³C{¹H} NMR (126 MHz, CDCl₃): 141.8 (s, 1 C), 137.4 (s, 1 C), 128.6 (t, J=5.1 Hz, 1 C), 127.9 (s, 1 C), 124.8 (t, J=4.9 Hz, 1 C), 118.8 (t, J=306.4 Hz, ArCF₂Br), −1.3 (s, 3 C, Me₃Si—C).

Compound 4k

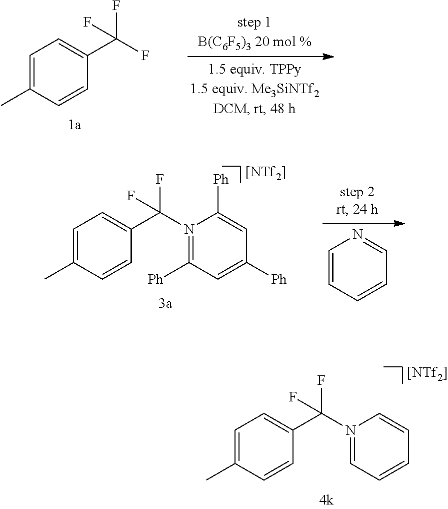

Compound 4k was prepared from 1a based on the protocol above where pyridine (60.4 μL, 0.75 mmol, 5.0 equiv.) was added in step 2 but a different method of purification was used.

After complete reaction ascertained by ¹⁹F NMR, the residue was washed with dry toluene (3×5 mL) and further dried in vacuo. The resultant brownish red sticky product was dissolved in DCM and treated with 10% NaHCO₃ (3×5 mL), followed by drying over Na₂SO₄. After removal of all volatiles, the residue was washed again with toluene (3×5 mL). The resultant brown material was dissolved in DCM, layered with n-hexane 1:5 (DCM:n-hexane) and stored at 5° C. Colourless crystals appeared after three days which were collected and dried to yield compound 4k (0.196 g, 65% yield). ¹H NMR (400 MHz, CDCl₃): δ_{H} 9.06-8.99 (m, 2H, Py—F), 8.77 (tt, J=7.8 Hz, J=1.3 Hz, 1H, Py—F), 8.31 (t, J=7.3 Hz, 2H, Py—F), 7.60 (d, J=8.5 Hz, 2H, Ar—f), 7.41 (d, J=8.7 Hz, 2H, Ar—F), 2.44 (s, 3H, Ar—Me); ¹⁹F NMR (377 MHz, CDCl₃): δ_{F}-76.9 (s, 2 F, Ar—CF₂—py), −79.0 (s, 6 F, —CF₃ of —NTf₂); ¹³C{¹H} NMR (101 MHz, CDCl₃): δ_{c} 150.4 (s), 145.2 (s), 140.1 (t, J=3.7 Hz), 130.7 (s), 129.4 (s), 126.1 (t, J=5.3 Hz), 124.6 (s), 124.1 (s), 121.3 (t, J=270.6 Hz), 119.5 (q, J=321.9 Hz), 21.4 (s); HRMS (ESI-TOF) m/z: 220.0896 for [C₁₃H₁₂F₂N]⁺ (calcd.: 220.0932).

Compound 41

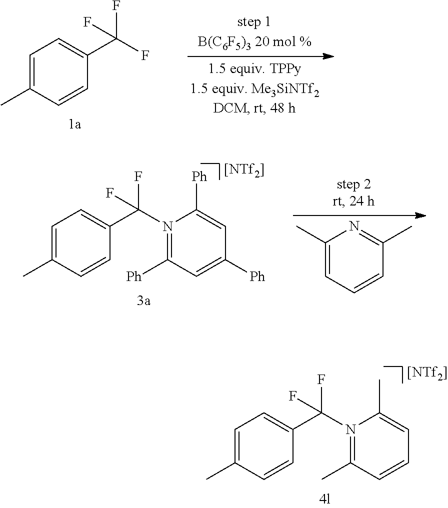

Compound 41 was prepared from 1a based on the protocol above where 2,6-dimethylpyridine (86.9 μL, 0.75 mmol, 5.0 equiv.) was added in step 2 but the crude product was purified using the purification method for 4k and a sticky oil was collected (0.109 g, 34% yield). ¹H NMR (400 MHz, CDCl₃): δ_{H} 8.45 (t, J=8.0 Hz, 1H, Lut-F), 7.93 (d, J=7.6 Hz, 2H, Lut-f), 7.40 (d, J=8.3 Hz, 2H, Ar—f), 7.30 (d, J=8.3 Hz, 2H, Ar—f), 2.82 (t, J=5.8 Hz, 6H, Lut-Me), 2.44 (s, 3H, Ar—Me); ¹⁹F NMR (377 MHz, CDCl₃): δ_{F}-56.4 (s, 2 F, Ar—CF₂—Lut), −78.9 (s, 6 F, —CF₃ of —NTf₂); ¹³C{¹H} NMR (101 MHz, CDCl₃): δ_{c} 156.9 (s), 148.4 (s), 145.0 (t, J=1.7 Hz), 131.0 (d, J=6.9 Hz), 126.6 (s), 125.6 (t, J=3.5 Hz), 125.5 (s), 121.3 (t, J=271.0 Hz), 119.8 (q, J=319.3 Hz), 24.8 (t, J=9.1 Hz), 21.4 (s); HRMS (ESI-TOF) m/z: 248.1208 for [C₁₅H₁₆F₂N]⁺ (calcd.: 248.1245).

Compound 4m

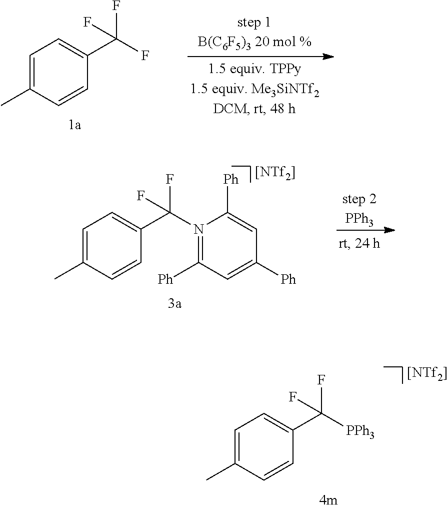

Compound 4m was prepared from 1a based on the protocol above where triphenyl phosphine (0.060 g, 0.23 mmol, 1.5 equiv.) was added in step 2 (0.175 g, 43% yield) but the crude product was purified using the purification method for 4k. ¹H NMR (400 MHz, CDCl₃): δ_{H} 8.03-7.92 (m, 3H, Ar—H of PPh₃), 7.84-7.72 (m, 6H, Ar—H of PPh₃), 7.67-7.53 (m Ar—H of PPh₃), 7.21 (d, J=8.1 Hz, 2H, Ar—H), 6.95 (d, J=7.7 Hz, 2H, Ar—H), 2.41 (s, 3H, Ar—Me); ¹⁹F NMR (377 MHz, CDCl₃): δ_{F}-78.7 (s, 6 F, —CF₃ of —NTf₂),-91.8 (d, J=114.2 Hz, 2 F, Ar—CF₂—P); ³¹P{¹H} NMR (162 MHz, CDCl₃): δₚ 24.8 (t, J=114.2 Hz, 1P, ArCF₂—P); ¹³C{¹H} NMR (101 MHz, CDCl₃): δ_{c} 144.7 (s), 137.03 (d, J=3.8 Hz), 135.0 (d, J=9.1 Hz), 131.0 (d, J=12.4 Hz), 129.9 (d, J=1.2 Hz), 127.2 (td, J=6.5 Hz, J=2.0 Hz), 121.7 (dt, J=270.6 Hz, J=91.0 Hz), 119.8 (q, J=329.4 Hz); 112.2 (d, J=76 Hz), 21.4 (s); HRMS (ESI-TOF) m/z: 403.1407 for [C₂₆H₂₂F₂P]⁺ (calcd.: 403.1421).

Compound 2a

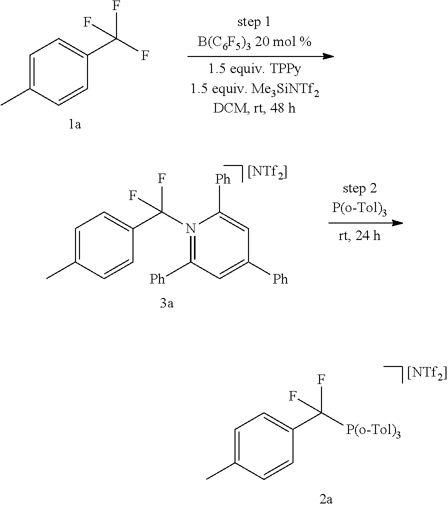

Compound 2a was prepared from 1a based on the protocol above where P(o-Tol)₃ ((0.070 g, 0.23 mmol, 1.5 equiv.) was added in step 2 but the crude product was purified using the purification method for 4k (0.266 g, 61% yield). NMR spectroscopic data was consistent and confirmed to be 2a which was isolated above.

### Example 7: Method for Synthesis of Difluorinated Compounds from Phosphonium Salt Using CS₂CO₃

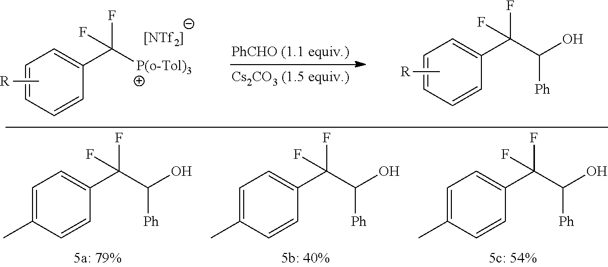

Into a 4 mL open PTFE top screw cap vial phosphonium salt (0.03 mmol, 1.0 equiv.) and Cs₂CO₃ (0.05 mmol, 1.5 equiv.) were taken. A solution of benzaldehyde (0.03 mmol, 1.1 equiv.) in 0.1 mL THF was added to the reaction vial and the reaction mixture was allowed to stir at 65° C. for 12 h. Reaction yield was assessed by ¹⁹F NMR with internal PhOCF₃ standard. Compounds 5b and 5c have been reported (Geri, J. B. et aL., J. Am. Chem. Soc. 2018, 140, 9404) whereas compound 5a was isolated.

Compound 5a

Compound 5a was prepared based on the protocol above as in example 7. Column chromatography purification with an eluent n-hexane/ethylacetate (95:5) gave 5a (0.045 g, 43% yield). ¹H NMR (400 MHz, CDCl₃) b_{H} 7.33-7.26 (m, 3H), 7.24-7.20 (m, 2H), 7.16-7.10 (m, 4H), 5.06 (t, J=10.1 Hz, 1H), 2.51 (s, 1H), 2.36 (s, 3H); ¹⁹F{¹H} NMR (377 MHz, CDCl₃): δ_{F}-105.9 (d, J=248.3 Hz), −106.7 (d, J=246.2 Hz); ¹³C{¹H} NMR (126 MHz, CDCl₃): 140.1 (t, J=1.8 Hz), 135.9 (t, J=2.3 Hz), 130.9 (t, J=26.1 Hz), 128.6, 128.0, 127.8, 127.79, 126.2 (t, J=6.2 Hz), 121.3 (t, J=247.8 Hz), 77.0 (t, J=31.0 Hz), 21.3; HRMS (APCI) m/z: 247.0935 for [M−H]⁺ (calcd.: 248.0940 for C₁₅H₁₄F₂O).

### Example 8: Method for Synthesis of Difluorinated Compounds from TPPy or Phosphonium Salt Via Catalyst Free Photoredox Coupling

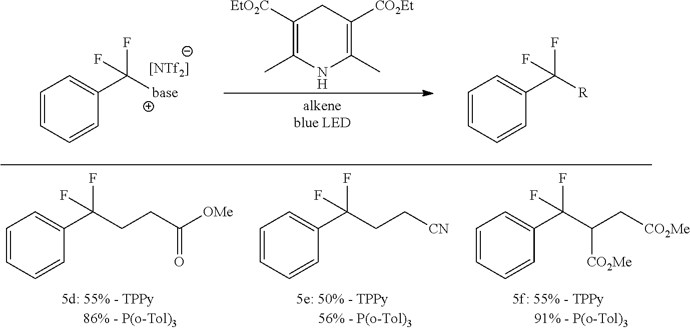

Using TPPy Salt:

Into a 4 mL open PTFE top screw cap vial 3b (0.01 mmol, 1.1 equiv.) and Hantzsch ester (0.03 mmol, 3.0 equiv.) were taken. Alkene (0.01 mmol, 1.0 equiv.) and DMA (0.5 M) added to the vial. The reaction vial was allowed to stir under blue LED irradiation at RT for 16 h. Reaction yield was assessed by ¹⁹F NMR with internal PhOCF₃ standard.

Using P(o-Tol)₃ salt:

Into a 4 mL open PTFE top screw cap vial 2b (0.06 mmol, 1.1 equiv.), Hantzsch ester (0.15 mmol, 3.0 equiv.) and K₂CO₃ (0.25 mmol, 5.0 equiv.) were added. Alkene (0.05 mmol, 1.0 equiv.) and DMF (0.25 M) added to the vial. The reaction vial is allowed to stir under blue LED irradiation at 40° C. for 16 h. Reaction yield was assessed by ¹⁹F NMR with internal PhOCF₃ standard.

### Example 9: Method for One-Pot Hydrodefluorination Via TPPy or Phosphonium Salt Using TPPy Salt

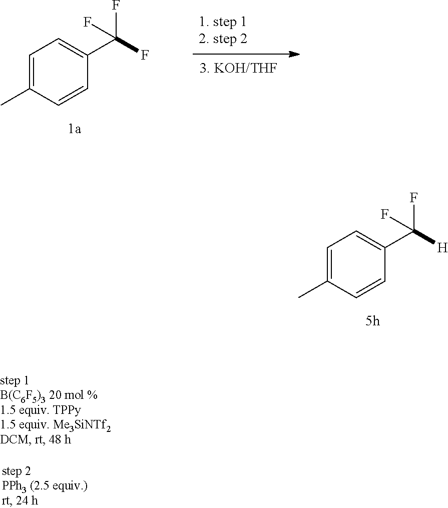

Steps 1-2 were followed as described in example 4. Complete reaction in step 2 was confirmed by ¹⁹F NMR analysis and DCM was removed under vacuum. The residue was dissolved in 0.4 mL of THF and 0.4 M KOH solution (0.5 mL) was transferred into the solution. After stirring for 10 minutes, reaction yield was assessed by ¹⁹F NMR with an internal PhOCF₃ standard (>95% yield).

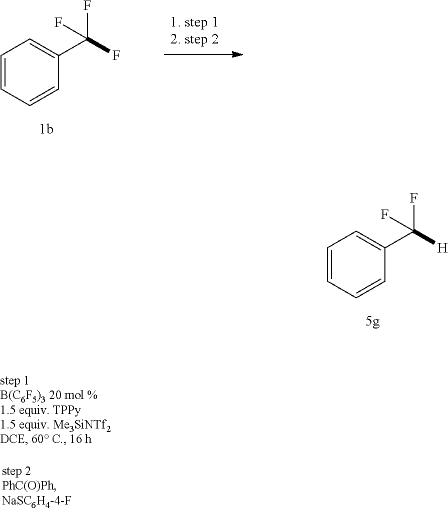

In a 4 mL open PTFE top screw cap vial BCF (0.03 mmol, 20 mol %), 1b (0.15 mmol, 1.0 equiv.) and Me₃SiNTf₂ (0.23 mmol, 1.5 equiv.) were added. A solution of TPPy (0.23 mmol, 1.5 equiv.) in 300 μL dry 1,2-DCE was added to the vial. The reaction mixture was stirred for 16 h at 60° C. NaS—C₆H₄-4-F (0.75 mmol, 5 equiv.) and PhC(O)Ph (0.17 mmol, 1.2 equiv.) were added to the reaction mixture. The reaction was allowed to stir at RT for 16 h. Reaction yield was assessed by ¹⁹F NMR with internal PhOCF₃ standard (90%).

Using P(o-Tol)₃ Salt:

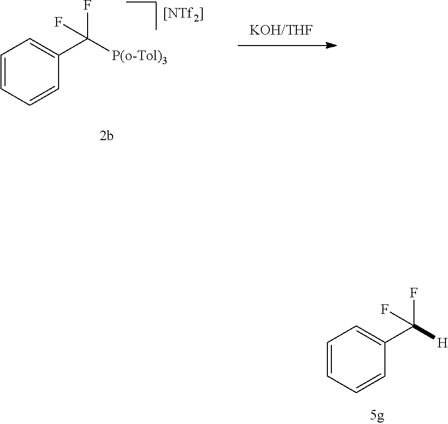

Compound 2b (0.15 mmol) was dissolved in THF (0.4 mL). 0.4 M KOH solution (0.5 mL) was transferred into the solution. After stirring for 10 minutes, yield was assessed by ¹⁹F NMR with an internal PhOCF₃ standard (>95% yield).

### Example 10: Method for [¹⁸F]-Fluoride Substitution of TPPy-Salt

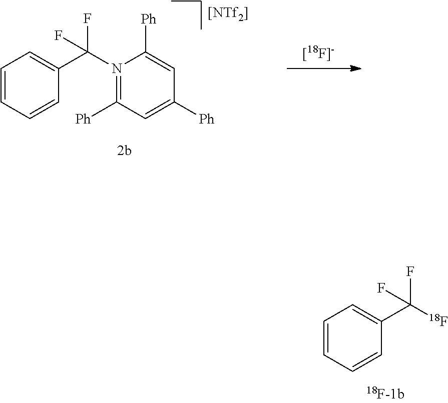

[¹⁸F]-fluoride was produced in a PET tracer 800 cyclotron. Resulted [¹⁸F]-fluoride was trapped on a standard commercially available QMA cartridge (Waters, Sep-Pak Light, Accell Plus QMA Carbonate) while the cartridge conditioned with H₂O (10 mL). Further an [¹⁸F]-fluoride elution cocktail prepared from a solution of tetraethylammonium bicarbonate (4.5 mg, 24 μmol) in H₂O (0.1 mL) and in CH₃CN (1.0 mL). The cocktail was eluted in extracting [¹⁸F]-fluoride from the QMA cartridge into a reaction vial. The eluted mixture with [¹⁸F]-fluoride treated for drying under vacuum with a stream of N₂ (350 mL/min) while the vial heated at 100° C. for 5 min. The drying process was repeated for second time with additional CH₃CN (1.0 mL). Subsequently, the resulted residue was extracted in CH₃CN (1.0 mL) and transferred to another vial, dried again with previously adopted procedure and sealed the vial with a PTFE cap. A solution of 2b (3 mg, 4.2 μmol) in 1,2-DCE (0.1 mL) was prepared under inert gas in a 1.5 mL vial sealed with a PTFE cap. The yellowish-green solution was added to the previous [¹⁸F]-fluoride reaction vial. The vial was allowed to warm on a pre-heated hot plate at 80° C. for 5 min.

Following completion of heating, the reaction mixture was diluted with CH₃CN/H₂O (1:1) (3.0 mL) and the resultant solution was injected into a gradient semi-prep HPLC column (Phenomenex Luna 5 μm C18(2) 100 Å LC column 250×10 mm, Pump A H₂O and Pump B CH₃CN) for isolation using gradient method with mobile phase CH₃CN:H₂O. The product fraction collected at 15 min affording activity of product fraction 60 MBq (non-decay corrected). Following isolation of pure fraction, 1 mL of the product fraction was injected into the analytical HPLC column (Phenomenex Luna 5 μm C18(2) 100 Å LC column 250×4.6 mm, Pump A H₂O and Pump B CH₃CN) for characterization resulting activity concentration 6.52 MBq/mL (non-decay corrected) and molar concentration of the [¹⁸F]-fluoride incorporated product obtained from a calibration curve is 0.03486 μmol/mL. The radiochemical purity of the product fraction is 88% and it is giving radiochemical yield 5.312% (decay corrected) (FIGS. 4 and 5). Radioactivity measurements were made with a CRC 55tPET dose calibrated.

Compound 1a-¹⁸F

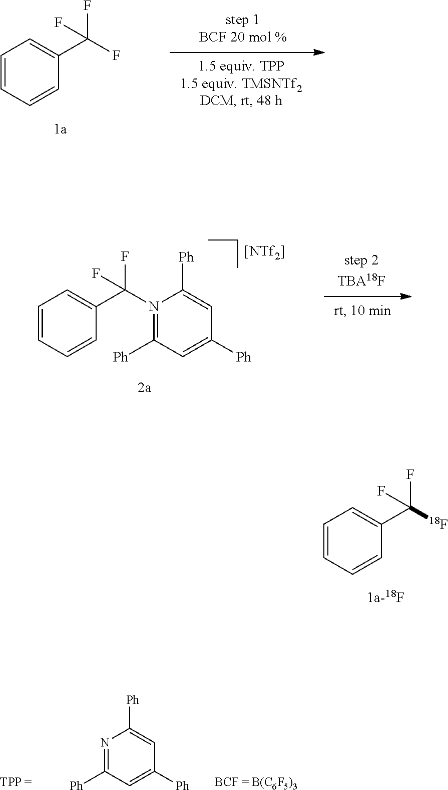

Compound 1a-¹⁸F was prepared from 1a based on the protocol above, where TBA¹⁸F was added in step 2 and the reaction vial was stirred at RT for 10 min in step 2. It is noted that compound 2a's formation is described hereinbefore and the same protocol may be used here. The results were similar to Compound 1b-¹⁸F.

Fluoxetine-¹⁸F

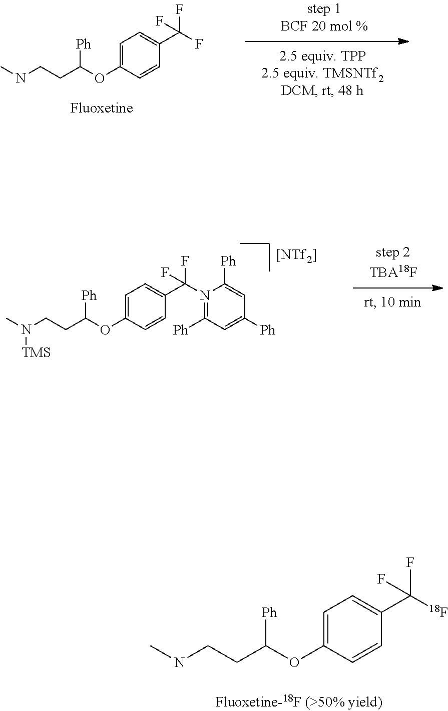

Fluoxetine-¹⁸F was prepared from Fluoxetine based on the protocols described above via a TMS-protected intermediate. It has subsequently been discovered that the trimethylammonium intermediate species (i.e. the NMe group is N(Me)₃*group) may be used to obtain a higher yield.

### Example 11: Method for Large Scale Syntheses and Isolation of TPPy Salts from Difluorinated Compounds

Compound 6c

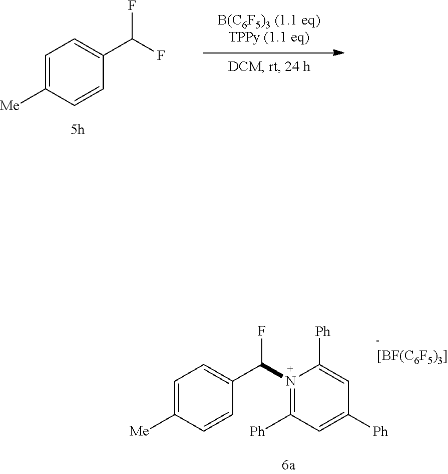

Into a 20 mL screw cap vial, B(C₆F₅)₃(0.159 g, 0.31 mmol, 1.1 eq.) and TPPy (0.096 g, 0.31 mmol, 1.1 eq.) were dissolved in DCM (5 mL). After addition of 5h (0.040 g, 0.28 mmol, 1.0 eq.), the yellowish-orange reaction mixture was allowed to stir at RT for 24 h. All volatiles were removed under vacuum. The residue was washed with n-hexane (3×5 mL) and further dried in vacuo. The sticky solid was dissolved in toluene (1 mL) a layer of n-hexane (5 mL) was added to it. The mixture was allowed to agitate, during which time a white solid crashed out. This process was repeated twice. Finally, all volatiles were removed in vacuo and the resultant solid material was dissolved in DCM and layered with n-hexane 1:5 (DCM:n-hexane) and stored at 5° C. White crystals appeared overnight, and were collected and dried to yield compound 6a (0.163 g, 60% yield). ¹H NMR (400 MHz, CD₂Cl₂) 5 8.13 (s, 1H), 7.93-7.43 (m, 7H), 6.78 (d, J=8.0 Hz, 1H), 6.45 (d, J=8.2 Hz, 1H), 2.22 (s, 1H); ¹⁹F NMR (377 MHz, CD₂Cl₂) δ-135.40 (dt, J=24.0, 10.4 Hz), −139.85 (d, J=45.8 Hz, ArCHF-TPPy), −165.49,-169.61 (m), −190.1; ¹⁹F{¹H} NMR (377 MHz, CD₂Cl₂) δ-135.40,-139.85 (s, ArCHF-TPPy), −162.39,-165.28,-170.65 (m), −190.06; ¹³C{¹H}NMR (101 MHz, CD₂Cl₂) 5 159.04, 158.51, 147.97 (d, J=241.0 Hz), 140.35, 139.95 (d, J=244.9 Hz), 136.44 (d, J=241.2 Hz), 133.80, 132.75, 132.21, 131.95, 130.12, 129.54, 129.47, 129.33 (d, J=1.6 Hz), 129.25, 129.21, 128.39, 127.12, 123.68 (d, J=6.9 Hz), 101.49 (d, J=224.9 Hz, ArCHF-TPPy), 20.80; HRMS (ESI-TOF) m/z: 430.1967 for [C₃₁H₂₅FN]⁺ (calcd.: 430.1966).

Compound 6b

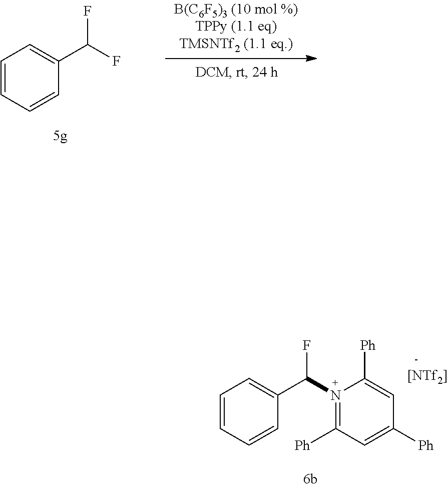

Into a 20 mL screw cap vial, B(C₆F₅)₃(0.031 g, 0.06 mmol, 10 mol %), Me₃SiNTf₂ (0.700 g, 1.98 mmol, 1.1 eq.) and TPPy (0.609 g, 1.98 mmol, 1.1 eq.) were dissolved in DCM (3.5 mL). Difluoride 5g (0.231 g, 1.80 mmol, 1.0 eq.) was added to the reaction mixture and the reaction mixture was stirred at RT for 24 h. All volatiles were removed in vacuo. The resultant yellowish-orange material was dissolved in DCM and treated with 0.1% NaHCO₃ (3×5 mL). Followed by drying over NaSO₄ and removal of all volatiles, the residue was washed with toluene (3×5 mL). The yellowish solid was dissolved in DCM and layered with n-hexane 1:5 (DCM:n-hexane) and stored at 5° C. Pale yellow crystals appeared after two days, which were collected and dried to afford compound 6b (0.870 g, 67% yield). ¹H NMR (400 MHz, CD₂Cl₂): 5 8.15 (s, 2H), 8.02-7.33 (m, 15H), 7.25-7.19 (m, 1H), 7.15-7.07 (m, 2H), 6.66-6.59 (m, 2H); ¹⁹F NMR (377 MHz, CD₂Cl₂): ¹⁹F NMR (377 MHz, CD₂Cl₂) δ-79.39,-140.06 (d, J=46.1 Hz, ArCHF-TPPy); ¹⁹F{¹H} NMR (377 MHz, CD₂Cl₂): ¹⁹F NMR (377 MHz, CD₂Cl₂) δ-79.39,-140.06 (s, ArCHF-TPPy); ¹³C{¹H} NMR (101 MHz, CD₂Cl₂): ¹³C NMR (101 MHz, CD₂Cl₂) 5 158.97, 158.86, 133.73, 133.00, 132.39 (d, J=22.3 Hz), 132.15, 131.90, 130.14, 129.78, 129.26, 128.87 (d, J=1.8 Hz), 128.74, 127.31, 123.79 (d, J=7.1 Hz), 119.96 (q, J=322.3 Hz), 101.05 (d, J=225.1 Hz, ArCHF-TPPy); HRMS (ESI-TOF) m/z: 416.1796.for [C₃₀H₂₃FN]⁺ (calcd.: 416.1809).

Compound 6c

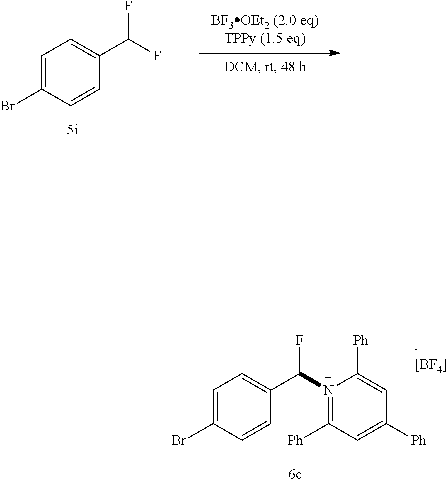

BF₃·OEt₂ (0.354 g, 2.50 mmol, 2.0 eq.) was added to a solution of TPPy (0.527 g, 1.87 mmol, 1.5 eq.) and 5i (0.258 g, 1.25 mmol, 1.0 eq.) in dry DCM (5.5 mL). The reaction mixture was allowed to stir at RT for 48 hours. All volatiles were removed in vacuo. The resultant yellowish sticky compound was dissolved in DCM and treated with 0.1% NaHCO₃ (3×5 mL). Followed by drying over NaSO₄ and removal of all volatiles, the residue was washed with toluene (3×5 mL). The yellowish solid was dissolved in DCM and layered with n-hexane 1:5 (DCM:n-hexane) and stored at −20° C. Pale yellow powder appeared after 12 hours, which was collected and dried to afford compound 6c (0.639 g, 88% yield). ¹H NMR (500 MHz, CD₂Cl₂) 6 8.12 (s, 2H), 8.01-7.35 (m, 14H), 7.24 (d, J=8.2 Hz, 3H), 6.64 (d, J=8.2 Hz, 2H); ¹⁹F NMR (377 MHz, CDCl₃) 5-139.21 (d, J=45.6 Hz, ArCHF-TPPy),-152.48; ¹⁹F{¹H} NMR (377 MHz, CDCl₃) 5-139.21,-152.49; ¹³C{¹H} NMR (126 MHz, CD₂Cl₂) δ159.15, 158.52, 133.48, 133.44, 132.33, 131.83, 131.53, 131.34, 130.03, 129.52, 129.19, 128.84, 127.79, 125.89 (d, J=7.0 Hz), 124.01, 100.51 (d, J=223.8 Hz, ArCHF-TPPy); HRMS (ESI-TOF) m/z: 490.0912 for [C₃₀H₂₂BrFN]⁺ (calcd: 494.0914).

Compound 6d

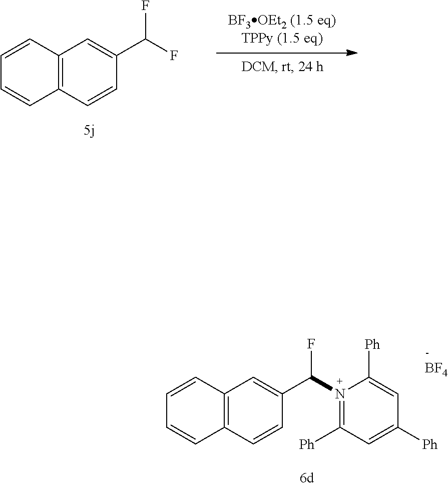

BF₃·OEt₂ (0.119 g, 0.84 mmol, 1.5 eq.) was added to a solution of TPPy (0.258 g, 0.84 mmol, 1.5 eq.) and 5j (0.100 g, 0.56 mmol, 1.0 eq.) in dry DCM (2.5 mL). The reaction mixture was allowed to stir at RT for 48 hours. All volatiles were removed in vacuo and the residue was washed with dry n-hexane (3×5 mL) and further dried in vacuo. The brown solid was dissolved in DCM and layered with n-hexane 1:5 (DCM:n-hexane) and stored at −20° C. Colourless powder appeared after 12 hours, which was collected and dried to afford compound 6d (0.170 g, 55% yield). ¹H NMR (400 MHz, CDCl₃) δ 8.06 (s, 3H), 7.98-7.93 (m, 3H), 7.85-7.83 (m, 1H), 7.74-7.50 (m, 10H), 7.48-7.35 (m, 7H), 6.94 (dd, J=8.7, 1.9 Hz, 1H); ¹⁹F NMR (377 MHz, CDCl₃) 5-138.94 (d, J=46.2 Hz, ArCHF-TPPy), −152.12; ¹⁹F{¹H} NMR (377 MHz, CDCl₃) 5-138.94,-152.12; ¹³C{¹H} NMR (126 MHz, CDCl₃) δ 159.32, 158.15, 133.96, 133.00, 132.83, 132.54, 132.36, 131.34, 129.80, 129.73, 129.55, 129.23, 128.98, 128.92, 128.60, 128.17, 127.61, 127.41 (d, J=10.3 Hz), 126.87, 124.95 (d, J=7.3 Hz), 120.48 (d, J=7.0 Hz), 101.23 (d, J=222.5 Hz, ArCHF-TPPy); HRMS (ESI-TOF) m/z: 466.1974 for [C₃₄H₂₅FN]⁺ (calcd.: 466.1966).

### Example 12: Method for NMR-Scale Functionalization of [RCHFTPPy]⁺ Salts

All the Experiments Described Below were Carried Out Under N₂ Atmosphere.

Procedure C

Into a 4 mL open PTFE top screw cap vial equipped with a stir bar, TBAF.xH₂O (0.04 mmol, 1.2 equiv.) and TPPy salt 6b (0.03 mmol, 1.0 equiv.) were taken in 1,2-DCB (150 μL). The reaction vial was allowed to stir for 5 min with a preheated oil bath at 150° C. The reaction yield was assessed by ¹⁹F NMR analysis with an internal standard, Ada-F. The exact conditions and characterising information for the resulting compounds are provided below.

Procedure D

Into a 4 mL open PTFE top screw cap vial equipped with a stir bar, TBAB (0.04 mmol, 1.2 equiv.) and a TPPy salt selected from 6c, 6d (0.03 mmol, 1.0 equiv.) were taken in 1,2-DCB (150 μL). The reaction vial was allowed to stir 16 hours at room temperature. The reaction yield was assessed by ¹⁹F NMR analysis with an internal standard, Ada-F. The exact conditions and characterising information for the resulting compounds are provided below.

Procedure E

Into a 4 mL open top PTFE screw cap vial, PhenNi(OAc)₂·xH₂O (3.6 mg, 0.01 mmol, 20 mol %), TPPy salt 6c (0.05 mmol, 1.0 equiv.), phenyl boronic acid (0.15 mmol, 3.0 equiv.), and K₃PO₄ (36.1 mg, 0.17 mmol, 3.4 equiv.) were added. Following that, dioxane (300 μL) was transferred to the reaction vial. The reaction mixture was allowed to stir at 60° C. for 18 h. Reaction yield was assessed by ¹⁹F NMR with internal PhF standard. The characterising information for the resulting compound are provided below.

Compound 5q

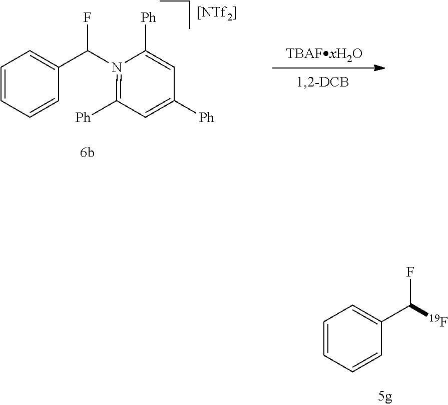

Compound 5g was prepared from 6b based on procedure C described above (75% yield). Characterising data matched literature reports: A. Haas, M. Spitzer, M. Lieb, Chem. Ber. 1988, 121, 1329-1340.

Compound 7a

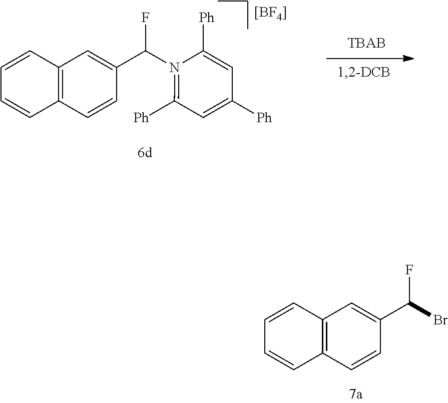

Compound 7a was prepared from 6d based on procedure D described above (85% yield).

¹⁹F NMR (377 MHz, CH₂Cl₂) δ-129.68 (d, J=50.0 Hz, ArCHF-TPPy); ¹⁹F{¹H} NMR (377 MHz, CH₂Cl₂) δ-129.68 (s, ArCHF-TPPy).

Compound 7b

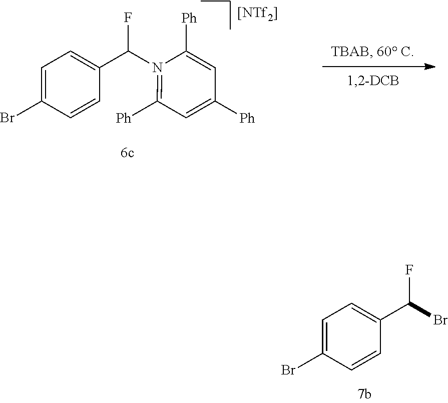

Compound 7b was prepared from 6c based on procedure D described above except the reaction was stirred at 60° C. for 16 hours (89% yield). Reference: W. Huang, X. Wan, Q. Shen, Org. Lett. 2020, 22, 4327-4332.

Compound 7d

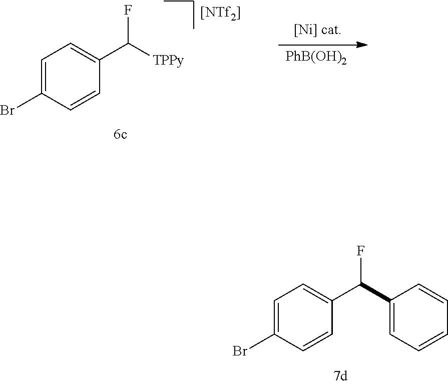

Compound 7d was prepared from 6c based on procedure E described above. Reaction yield was assessed by ¹⁹F NMR with internal PhF standard (59% yield) and ¹⁹F NMR chemical shifts of the formed 7d was confirmed by comparison to the literature (D. Bethell, et aL., Tetrahedron Lett. 1977, 18, 1447).

### Example 13: Method for NMR-Scale Synthesis of Monofluorinated Compound from TPPy Salt Via Catalyst Free Photoredox Coupling

The experiment was carried out under N₂ atmosphere. Into a 4 mL open PTFE top screw cap vial, TPPy salt 6b (0.017 mmol, 1.1 equiv.), Hantzsch ester (0.045 mmol, 3.0 equiv.) and K₂CO₃ (0.075 mmol, 5.0 equiv.) were taken. Followed by addition of DMF (150 μL), methyl acrylate (0.015 mmol, 1.0 equiv.) was transferred to the reaction vial. The vial is allowed to stir under blue LED irradiation at 40° C. for 18 h. Reaction yield was assessed by ¹⁹F NMR with an internal PhOCF₃ standard.

Compound 7c

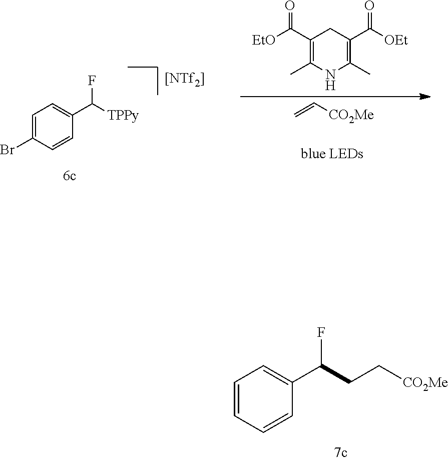

Compound 7c was prepared from 6b based on the protocol above. Reaction yield was assessed by ¹⁹F NMR with an internal PhOCF₃ standard (40% yield) and ¹⁹F NMR chemical shifts of the formed 7c was confirmed by comparison to the literature (W. Liu, et aL., Angew. Chem. Int. Ed. 2013, 52, 6024).

1. A salt of formula I: 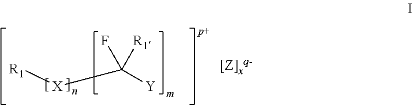
wherein:
m and p are 1 to 6;
n is 0 or 1;
q is 1 or 2 and o is 1 to 6, where Z is one or more counterions that balance a charge p+;
X, when present, is O, S or NR^{2a}R^{2b};
Y is —NR^{3a}R^{3b}R^{3c} or —PR^{4a}R^{4b}R^{4c};
R¹ is selected from H, alkyl, alkenyl, alkynyl, heterocyclic, aryl, or heteroaryl, which groups are unsubstituted or substituted by one or more groups selected from:
(a) halo;
(b) CN;
(c) C₁₋₆ alkyl, C₂₋₆ alkenyl, C₂₋₆ alkynyl (which latter three groups are unsubstituted or substituted by one or more substituents selected from halo, nitro, CN, C₁₋₆ alkyl, C₂₋₆ alkenyl, C₂₋₆ alkynyl (which latter three groups are unsubstituted or substituted by one or more substituents selected from OH, ═O, halo, C₁₋₄ alkyl and C₁₋₄ alkoxy), Cy^{l}(which Cy^{l} group is unsubstituted or is substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{5a}, S(O)_{q}R^{5b}, S(O)₂NR^{5c}R^{5d}, NR^{5e}S(O)₂R^{5f}, NR^{5g}R^{5h} aryl and Het¹);
(d) Cy² (which Cy² group is unsubstituted or is substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{6a}, S(O)_{q}R^{6b}, S(O)₂NR^{6c}R^{6d}, NR^{6e}S(O)₂R^{6f}, NR^{6g}R^{6h} aryl and Het²),
(e) Het^{a} (which Het^{a} group is unsubstituted or substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{7a}, S(O)_{q}R^{7b}, S(O)₂NR^{7c}R^{7d}, NR^{7e}S(O)₂R^{7f}, NR^{7g}R^{7h}, aryl and Het³);
(f) OR^{8a};
(g) S(O)_{q}R^{8b};
(h) S(O)₂NR^{8c}R^{8d};
(i) NR^{8e}S(O)₂R^{8f};
(j) NR^{8g}R^{8h},
R^{3a} to R^{3c} and R^{4a} to R^{4c} are each independently selected from aryl or heteroaryl, or R^{3a} to R^{3c} together form a pyridinium ring, which groups are unsubstituted or substituted by one or more groups selected from:
(a) halo;
(b) CN;
(c) C₁₋₆ alkyl, C₂₋₆ alkenyl, C₂₋₆ alkynyl (which latter three groups are unsubstituted or substituted by one or more substituents selected from halo, nitro, CN, C₁₋₆ alkyl, C₂₋₆ alkenyl, C₂₋₆ alkynyl (which latter three groups are unsubstituted or substituted by one or more substituents selected from OH, ═O, halo, C₁₋₄ alkyl and C₁₋₄ alkoxy), Cy³ (which Cy³ group is unsubstituted or is substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{9a}, S(O)_{q}R^{9b}, S(O)₂NR^{9c}R^{9d}, NR^{9e}S(O)₂R^{9f}, NR^{9g}R^{9f}, aryl and Het⁴);
(d) Cy⁴ (which Cy⁴ group is unsubstituted or is substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{10a}, S(O)_{q}R^{10b}, S(O)₂NR^{10c}R^{10d}, NR^{10e}S(O)₂R^{10f}, NR^{10g}R^{10h} aryl and Het⁵),
(e) Het^{b} (which Het^{b} group is unsubstituted or substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy), OR^{12a}, S(O)_{q}R^{12b}S(O)₂NR^{12c}R^{12a}, NR^{12d}S(O)₂R^{12f}, NR^{12g}R^{12h} aryl and Het⁶);
(f) OR^{13a};
(g) S(O)_{q}R^{13b};
(h) S(O)₂NR^{13c}R^{13d};
(i) NR^{8e}S(O)₂R^{3f};
(j)NR^{13g}R^{13h},
R^{2a}, R^{2b}, R^{5a} to R^{5f}, R^{6a} to R^{6f}, R^{7a} to R^{7h}, R^{8a} to R^{8h}, R^{9a} to R^{9f}, R^{10a} to R^{10h}, R^{11a} to R^{11h}R^{12a} to R^{12h}, and R^{13a} to R^{13h} independently represent, at each occurrence, H, C₁₋₆ alkyl, C₂₋₆ alkenyl, C₂₋₆ alkynyl (which latter three groups are unsubstituted or are substituted by one or more substituents selected from halo, nitro, ═O, C(O)OC₁₋₄ alkyl, CN, C₁₋₆ alkyl, C₂₋₆ alkenyl, C₂₋₆ alkynyl, C₃₋₆ cycloalkyl (which latter four groups are unsubstituted or are substituted by one or more substituents selected from OH, ═O, halo, C₁₋₄ alkyl and C₁₋₄ alkoxy), OR^{14a}, S(O)_{q}R^{14b}, S(O)₂NR^{14C}R^{14d}, NR^{14e}S(O)₂R^{14f}, NR¹⁴⁹R^{14h} aryl and Het⁷), C₃₋₁₀ cycloalkyl, or C₄₋₁₀ cycloalkenyl (which latter two groups are unsubstituted or are substituted by one or more substituents selected from halo, OH, ═O, C₁₋₆ alkyl and C₁₋₆ alkoxy) or Het^{c}, or R^{2a} and R^{2b}, R^{5-14c} and R⁵⁻¹⁴, and R^{5-14g} and R^{5-14h} represent, together with a nitrogen atom to which they are attached, a 3— to 10-membered heterocyclic ring that may be aromatic, fully saturated or partially unsaturated and which may additionally contain one or more heteroatoms selected from O, S and N, which heterocyclic ring is unsubstituted or are substituted by one or more substituents selected from halo, nitro, CN, C₁₋₆ alkyl, C₂₋₆ alkenyl, C₂₋₆ alkynyl (which latter three groups are unsubstituted or are substituted by one or more substituents selected from OH, ═O, halo, C₁₋₄ alkyl and C₁₋₄ alkoxy);
Het¹ to Het⁶, Het^{a} to Het^{c} independently represent a 4— to 14-membered heterocyclic groups containing one or more heteroatoms selected from O, S and N, which heterocyclic groups may comprise one, two or three rings and may be substituted by one or more substituents selected from ═O, halo, C₁₋₆ alkyl, which latter group is unsubstituted or is substituted by one or more substituents selected from halo, —OR^{15a},
—NR^{15b}R^{15c}, —C(O)OR^{15d} and —C(O)NR^{15e}R^{15f};
Cy¹ to Cy⁴, at each occurrence, independently represents a 3— to 10-membered aromatic, fully saturated or partially unsaturated carbocyclic ring;
R^{5a} to R^{15h} independently represent at each occurrence, H, C₁₋₄ alkyl, C₂₋₄ alkenyl, C₂₋₄ alkynyl which latter three groups are unsubstituted or are substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₄ alkyl and C₁₋₄ alkoxy), C₃₋₆ cycloalkyl, or C₄₋₆ cycloalkenyl (which latter two groups are unsubstituted or are substituted by one or more substituents selected from halo, OH, ═O, C₁₋₄ alkyl and C₁₋₄ alkoxy).

2. The salt of formula I according to claim 1, wherein:
m and p are 1 to 3;
n is 0 or 1;
q is 1 and o is l to 3; and
X, when present, is O or S.

3. The salt of formula I according to claim 1, wherein:
R¹ is selected from H, C₁₋₆ alkyl, C₂₋₆ alkenyl, C₂₋₆ alkynyl, heterocyclic, aryl, or heteroaryl, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, C₁₋₄ alkyl, C₂₋₄ alkenyl, C₂₋₄ alkynyl (which latter three groups are unsubstituted or substituted by one or more substituents selected from OH, ═O, halo, C₁₋₃ alkyl and C₁₋₃ alkoxy), Cy^{l}(which Cy^{l} group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{5a}, S(O)_{q}R^{5b}, S(O)₂NR^{5c}R^{5d}, NR^{5e}S(O)₂R^{5f}, NR^{5g}R^{5h} aryl and Het¹);
(d) Cy² (which Cy² group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{6a}, S(O)_{q}R^{6b}, S(O)₂NR^{6c}R^{6d}, NR^{6e}S(O)₂R^{6f}, NR^{6g}R^{6h} aryl and Het²),
(e) Het^{a} (which Het^{a} group is unsubstituted or substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{7a}, S(O)_{q}R^{7b}, S(O)₂NR^{7c}R^{7d}, NR^{7e}S(O)₂R^{7f}, NR^{7g}R^{7h}, aryl and Het³);
(f) OR^{8a};
(g) S(O)_{q}R^{8b};
(h) S(O)₂NR^{8c}R^{8d};
(i) NR^{8e}S(O)₂R^{8f};
(j) NR^{8g}R^{8h}.

4. The salt of formula I according to claim 3, wherein:
R¹ is selected from C₁₋₆ alkyl, aryl, or heteroaryl, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, unsubstituted C₁₋₄ alkyl, Cy^{l}(which Cy^{l} group is unsubstituted or is substituted by one or more substituents selected from halo, nitro, CN, unsubstituted C₁₋₄ alkyl, OR^{5a}, and NR^{5g}R^{5h});
(d) Cy² (which Cy² group is unsubstituted or is substituted by one or more substituents selected from halo, nitro, CN, unsubstituted C₁₋₄ alkyl, OR^{6a}, and NR^{6g}R^{6h}),
(e) Het^{a} (which Het^{a} group is unsubstituted or substituted by one or more substituents selected from halo, nitro, CN, unsubstituted C₁₋₄ alkyl, OR^{7a}, and NR⁷⁹R^{7h});
(f) OR^{8a};
(g) NR^{8g}R^{8h}, optionally, wherein
R¹ is selected from C₁₋₆ alkyl, phenyl, or pyridyl, which groups are unsubstituted or substituted by one or more groups as described in claim 1.

5. The salt of formula I according to claim 1, wherein:
R^{3a} to R^{3c} and R^{4a} to R^{4c} are each independently selected from aryl or heteroaryl, or R^{3a} to R^{3c} together form a pyridinium ring, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, C₁₋₄ alkyl, C₂₋₄ alkenyl, C₂₋₄ alkynyl (which latter three groups are unsubstituted or substituted by one or more substituents selected from OH, ═O, halo, C₁₋₃ alkyl and C₁₋₃ alkoxy), Cy³ (which Cy³ group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{9a}, S(O)_{q}R^{9b}, S(O)₂NR^{9c}R^{9d}, NR^{9e}S(O)₂R^{9f}, NR^{9g}R^{9f}, aryl and Het⁴);
(d) Cy⁴ (which Cy⁴ group is unsubstituted or is substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{10a}, S(O)_{q}R^{10b}, S(O)₂NR^{10c}R^{10d}, NR^{10e}S(O)₂R^{10f}, NR^{10g}R^{10h} aryl and Het⁵),
(e) Het^{b} (which Het^{b} group is unsubstituted or substituted by one or more substituents selected from halo, nitro, CN, C₁₋₄ alkyl, C₂₋₄ alkenyl, C₂₋₄ alkynyl (which latter three groups are unsubstituted or are substituted by one or more substituents selected from OH, ═O, halo, C₁₋₃ alkyl and C₁₋₃ alkoxy), OR^{12a}, S(O)_{q}R^{12b}, S(O)₂NR^{12c}R^{12a}, NR^{12e}S(O)₂R^{12f}, NR^{12g}R^{12h} aryl and Het⁶);
(f) OR^{13a};
(g) S(O)_{q}R^{13b};
(h) S(O)₂NR^{13c}R^{13d};
(i) NR^{8e}S(O)₂R^{3f};
(j)NR^{13g}R^{13h}.

6. The salt of formula I according to claim 5, wherein:
R^{3a} to R^{3c} and R^{4a} to R^{4c} are each independently selected from aryl or heteroaryl, or R^{3a} to R^{3c} together form a pyridinium ring, which groups are unsubstituted or substituted by one or more groups selected from:
(a) halo;
(b) CN;
(c) C₁₋₄ alkyl, C₂₋₄ alkenyl, C₂₋₄ alkynyl (which latter three groups are unsubstituted or substituted by one or more substituents selected from halo, nitro, CN, unsubstituted C₁₋₄ alkyl, Cy³ (which Cy³ group is unsubstituted or is substituted by one or more substituents selected from halo, nitro, CN, unsubstituted C₁₋₄ alkyl, OR^{9a}, and NR^{9g}R^{9h});
(d) Cy⁴ (which Cy⁴ group is unsubstituted or is substituted by one or more substituents selected from halo, nitro, CN, unsubstituted C₁₋₄ alkyl, OR^{10a}, S(O)_{q}R^{10b}, S(O)₂NR^{10c}R^{10d}, NR^{10e}S(O)₂R^{10f}, NR^{10g}R^{10h} aryl and Het⁵),
(e) Het^{b} (which Het^{b} group is unsubstituted or substituted by one or more substituents selected from halo, nitro, CN, unsubstituted C₁₋₄ alkyl, OR^{12a}, and NR^{12g}R^{12h});
(f) OR^{13a};
(g) NR^{13g}R^{13h}.

7. The salt of formula I according to claim 1, wherein, when present:
R^{2a} and R^{2b}, R^{5a} to R^{5f}, R^{6a} to R^{6f}, R^{7a} to R^{7h}, R^{8a} to R^{8h}, R^{9a} to R^{9f}, R^{10a} to R^{10h}, R^{11a} to R^{11b},R^{12a} to R^{12f}, and R^{13a} to R^{13h} independently represent, at each occurrence, H or C₁₋₄ alkyl (which is unsubstituted or is substituted by one or more substituents selected from halo, nitro, ═O, CN, unsubstituted C₁₋₄ alkyl, OR^{14a}, and NR¹⁴⁹R^{14h}) or
R^{2a} and R^{2b}, R⁵-^{14c} and R⁵⁻¹⁴d and R⁵-¹⁴⁹ and R^{5-14h} represent, together with the nitrogen atom to which they are attached, a 3— to 10-membered heterocyclic ring that may be aromatic, fully saturated or partially unsaturated and which may additionally contain one or more heteroatoms selected from O, S and N, which heterocyclic ring is unsubstituted or are substituted by one or more substituents selected from halo, nitro, CN, or C₁₋₆ alkyl.

8. (canceled)

9. The salt of formula I according to claim 1, wherein Y is
—NR^{3a}R^{3b}R^{3c}.

10. The salt of formula I according to claim 1, wherein Y is selected from: 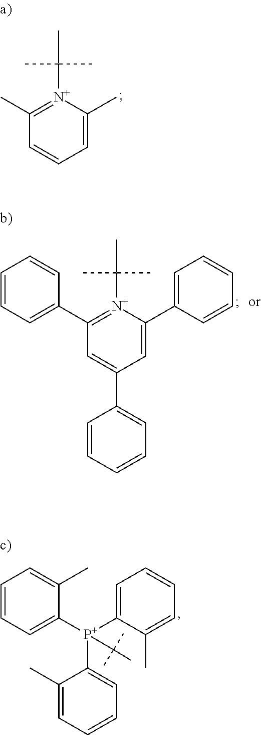
where dotted line represents a point of attachment to rest of molecule.

11. The salt of formula I according to claim 1, wherein Y is: 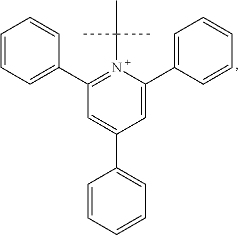
where dotted line represents a point of attachment to rest of molecule.

12. The salt of formula I according to claim 1, wherein:
(a) Z is selected from one or more of B⁻ (C₆F₅)₄, FB⁻ (C₆F₅)₃ or, N⁻ (SO₂CF₃)₂; and/or
(b) R¹′ is F.

13. The salt of formula I according to claim 1, selected from a list of:

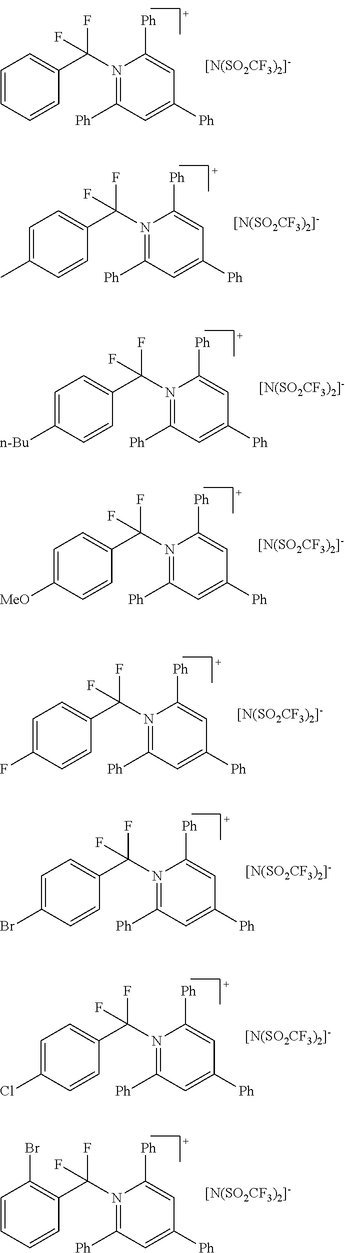

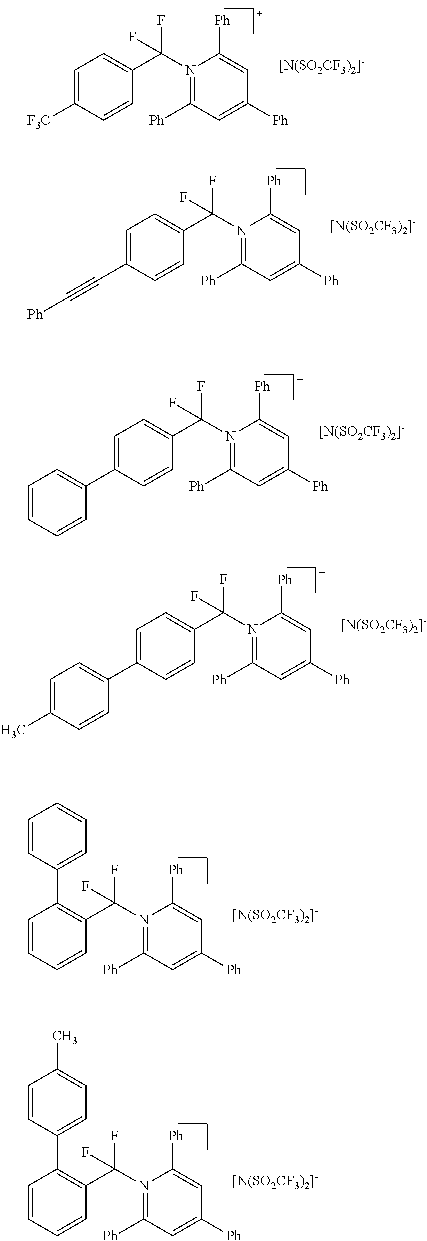

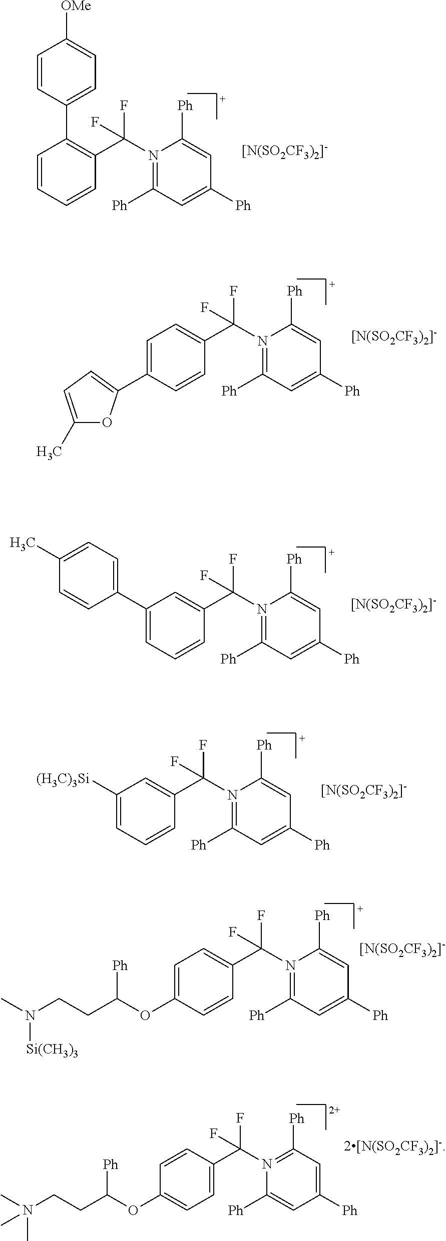

14. A method of forming a compound of formula I as described in claim 1, the method comprising a step of reacting a compound of formula II, 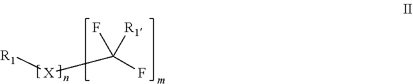
with a compound of formula IIIa or IIIb:
NR^{3a}R^{3b}R^{3c} IIIa; or
PR^{4a}R^{4b}R^{4c} IIIb,
in a presence of a catalyst and a counterion source, where n, m, R¹, R^{3a} to R^{3b} and R^{4a} to R^{4b} are as described in claim 1, provided that when R¹′ is H, aryl or alkyl, then reaction is with a compound of formula IIIa.

15. The method according to claim 14, wherein:
(a) the counterion source is selected from Li[B(C₆F₅)₄] or N-(trimethylsilyl)bis(trifluoromethanesulfonyl)imide; and/or
(b) the catalyst is selected from B(C₆F₅)₃.

16. A method of providing a difluorinated compound with or without an isotopic label, comprising a step of reacting a compound of formula I as described in claim 1, with a nucleophilic source compound with or without an isotopic label to form the difluorinated compound.

17. A one-pot method of providing a difluorinated compound with or without an isotopic label from a compound of formula II, the method 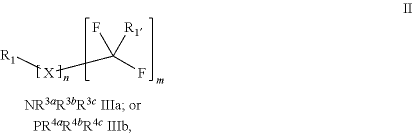
where n, m, R¹, R^{3a} to R^{3b} and R^{4a} to R^{4b} are as described in claim 1, comprising steps of:
(a) reacting a compound of formula II with a compound of formula IIIa or IIIb in a presence of a catalyst and a counterion source to provide a compound of formula I, provided that when R¹′ is H, aryl or alkyl, then the reaction is with a compound of formula IIIa, and the compound of formula I is as described claim 1; and
(b) reacting a compound of formula I as described in claim 1, with a nucleophilic source compound with or without an isotopic label to form the difluorinated compound.

18. (canceled)

19. A method of forming a difluorinated compound through nucleophilic difluorination, the method comprising a step of reacting a compound of formula I as described in claim 1 with a compound having an thioaldehyde group, a thioketone group or an aldehyde group, a ketone group or an imine group in a presence of an initiator compound to form a difluorinated compound, optionally wherein the initiator compound is selected from an inorganic base.

20. A method of forming either a difluorinated compound through a radical coupling reaction to an alkene, alkyne or hydrogen, the method comprising reacting a compound of formula I as described in claim 1 with an alkene or alkyne or hydrogen source in a presence of a radical initiator to generate the difluorinated compound.
